(19) United States
(12) Patent Application Publication
(10) Pub. No.: US 2024/0239844 A1
(43) Pub. Date: Jul. 18, 2024

(54) COMBINATIONS OF VIRAL PROTEINS, PEPTIDE SEQUENCES, EPITOPES, AND METHODS AND USES THEREOF

(71) Applicant: LA JOLLA INSTITUTE FOR IMMUNOLOGY, La Jolla, CA (US)

(72) Inventors: Erica Ollmann SAPHIRE, La Jolla, CA (US); Haoyng LI, La Jolla, CA (US)

(21) Appl. No.: 17/922,307

(22) PCT Filed: Apr. 30, 2021
(86) PCT No.: PCT/US2021/030325

§ 371 Date: Oct. 28, 2023

Related U.S. Application Data
(60) Provisional application No. 63/018,080, filed on Apr. 30, 2020.
(60) Provisional application No. 63/051,776, filed on Jul. 14, 2020.
(60) Provisional application No. 63/081,790, filed on Sep. 22, 2020.
(60) Provisional application No. 63/089,407, filed on Oct. 8, 2020.

(51) Int. Cl. C07K 14/005; A61K 39/00; A61K 39/12; A61K 39/215; C12N 15/86

(52) U.S. Cl. CPC C07K 14/005; A61K 39/12; A61K 39/215; C12N 15/86; A61K 2039/70; C12N 2710/14043; C12N 2740/15043; C12N 2760/14122; C12N 2760/14134; C12N 2760/20243; C12N 2770/20022; C12N 2770/20034

(57) ABSTRACT

Compositions and methods are disclosed for treating and/or preventing viral infections and related pathologies (e.g., Coronavirus and Ebola virus infections and related pathologies). Composition includes those with modified viral glycoproteins, wherein one or more portions of the modified viral glycoproteins are modified to comprise an antigen. Variously, the antigen can include a Coronavirus antigen or an Ebola virus antigen.

### CROSS-REFERENCE TO RELATED APPLICATIONS

This application claims priority to, and the benefit of, U.S. Provisional Patent Application Ser. No. 63/018,080, entitled “Combinations of Viral Proteins, Peptide Sequences, Epitopes, and Methods and Uses Thereof” and filed on Apr. 30, 2020; U.S. Provisional Patent Application Ser. No. 63/051,776, entitled “Combinations of Viral Proteins, Peptide Sequences, Epitopes, and Methods and Uses Thereof” and filed on Jul. 14, 2020; U.S. Provisional Patent Application Ser. No. 63/081,790, entitled “Combinations of Viral Proteins, Peptide Sequences, Epitopes, and Methods of Uses Thereof” and filed on Sep. 22, 2020; and U.S. Provisional Patent Application Ser. No. 63/089,407, entitled “Pan-Vaccine Platform for Combining Viral Proteins, Peptide Sequences, and Epitopes, and Methods of Uses Thereof” and filed on Oct. 8, 2020, the entire contents of each of which are incorporated herein by reference in their entirety.

### TECHNICAL FIELD

The present application relates to compositions of matter, processes and use of compositions of matter relating to viral proteins, peptides and epitopes, and particularly to viral proteins, peptides, and epitopes from the Coronaviridae and Filoviridae families of viruses.

### BACKGROUND

Effective countermeasures against pathogens that have recently emerged and are rapidly expanding are required. For example, effective countermeasures against the recent emergence and rapid expansion of the 2019-Novel Coronavirus (SARS-COV-2, a.k.a. COVID-19, 2019-nCOV, Wuhan-Hu-1, etc.) require the development of data and tools to understand and monitor viral spread and immune responses.

Effective countermeasures are also required against previously-known viral pathogens such as, for example, Lassa fever, which is caused by Lassa virus. In addition, effective countermeasures are also required against pathogens that pre-date the 2019-Novel Coronavirus outbreak, such as the continued concerns raised by viruses such as Ebola virus.

There remains an urgent need to address the fundamental gaps in the understanding of the immunology and pathogenesis related to these viruses so as to be able to develop more effective vaccines and/or treatment approaches.

### BRIEF DESCRIPTION OF THE FIGURES

In the present application:

FIG. 1 provides a summary of the technology described herein, and an example of one or more embodiments as set forth in the present disclosure.

FIG. 2 demonstrates that ZEBOV-GP-deltaMLD-SARS-COV-2-RBD is recognized by both anti-EBOV-GP neutralizing antibodies and soluble human ACE2 receptor, and is an example of one or more embodiments as set forth in the present disclosure.

FIG. 3 demonstrates that ZEBOV-GP-deltaMLD-mCherry is recognized by anti-EBOV-GP neutralizing antibodies and mCherry fluorescent protein is functionally displayed, and is an example of one or more embodiments as set forth in the present disclosure.

FIG. 4 demonstrates three vaccine vectors displaying ZEBOV-GP-deltaMLD-SARS-CoV-2-RBD. For baculovirus surface display, the signal peptide (SP) and cytoplasmic domain (C-ter) of ZEBOV GP were replaced by the counterparts of baculovirus GP64.

FIG. 5 demonstrates ELISA binding assay of baculovirus and monoclonal antibodies. Baculoviruses displaying three different GPs respectively. Anti-Ebola neutralizing antibody (ADI-15878; FIG. 5a) and anti-SARS-COV-2-RBD conformational antibody (CR3022; FIG. 5b) were used to verify the confirmation of ZEBOV-GP and RBD.

FIG. 6 depicts an immunization flow chart for recombinant baculovirus displaying ZEBOV-GP or ZEBOV-GP-deltaMLD-SARS-COV-2-RBD.

FIGS. 7a-7d demonstrates results showing total IgG against ZEBOV-GP or SARS-CoV-2-spike in mice sera immunized with recombinant baculovirus.

FIG. 8 demonstrates results showing neutralizing activity against rVSV-based SARS-CoV-2 pseudovirus in sera of recombinant baculovirus vaccination.

FIG. 9 demonstrates results of immunofluorescence assays. GPs were expressed on the surface of HEK293T cells. Pan-Ebola neutralizing antibody (ADI-15878); anti-MARV neutralizing antibody (MR191) and anti-SARS-COV-2-RBD and SARS-COV-1-RBD conformational antibody (CR3022) and anti-MERS-COV-RBD neutralizing antibody (m336) were used to verify the conformation of GPs and RBDs.

FIG. 10 depicts a DNA sequence of a Zaire ebolavirus (Mayinga strain) construct, namely: ZEBOV-GP-deltaMLD-SARS-COV-2-RBD (SEQ ID NO: 1).

FIG. 11 depicts a DNA sequence of a Marburg virus (Angola strain) construct, namely: MARV-GP-deltaMLD-SARS-COV-2-RBD (SEQ ID NO: 2).

FIG. 12 depicts a DNA sequence of a Sudan Ebola virus (Gulu strain) construct, namely: SUDV-GP-deltaMLD-SARS-COV-1-RBD (SEQ ID NO: 3).

FIG. 13 depicts a DNA sequence of a Bundibugyo ebolavirus construct, namely: BDBV-GP-deltaMLD-MERS-COV-RBD (SEQ ID NO: 4).

FIG. 14 depicts a DNA sequence of a Zaire ebolavirus (Mayinga strain) construct, namely: ZEBOV-GP-deltaMLD-mCherry (SEQ ID NO: 5).

FIG. 15 depicts a DNA sequence for a plasmid construct, namely: pVenus-ZEBOV-GP-deltaMLD-SARS-COV-2-RBD (SEQ ID NO: 6).

FIG. 16 depicts a DNA sequence for a plasmid construct, namely: pFastBac-GFP-ZEBOV-GP-deltaMLD-SARS-COV-2-RBD (SEQ ID NO: 7).

### SUMMARY

This Summary is provided to introduce a selection of concepts in a simplified form that are further described below in the Detailed Description. This Summary is not intended to identify key aspects or essential aspects of the claimed subject matter.

As embodied and broadly described herein, the present disclosure relates to a composition comprising a viral glycoprotein, wherein one or more portions of the viral glycoprotein are modified to comprise, consist, or consist essentially of an antigen. In certain embodiments, the viral glycoprotein and the antigen are from different sources. In other embodiments, the composition further comprises a viral vector. In still other embodiments, the viral vector is a lentiviral vector. In other embodiments, the viral vector comprises a lentiviral vector that, when expressed to produce lentiviral particles, is capable of being displayed on a baculovirus surface. In still other embodiments, the viral vector is a recombinant vesicular stomatitis virus (rVSV). In some embodiments, the VSV is a non-exotic Indiana serotype strain. In some embodiments, the viral vector is a recombinant adenovirus. In some embodiments, the viral vector is a recombinant baculovirus. In some embodiments, and in respect of the baculovirus vector disclosed herein, the receptor binding domain (RBD) of SARS-COV-2 on the chimeric glycoprotein (GP) is better able to facilitate the virion uptake by antigen-presenting (APCs) (e.g., lung macrophages and dendritic cells etc.), since ACE2 is universally expressed in different cell types, including APCs. It will be appreciated that antigen uptake by APCs is a crucial step for generating an effective immune response.

In certain embodiments, the viral glycoprotein comprises a glycoprotein or mutant thereof from the family Filoviridae. In certain embodiments, the viral glycoprotein comprises a glycoprotein or mutant thereof from the filovirus genus. In certain embodiments, the viral glycoprotein comprises a glycoprotein or mutant thereof from the Marburg or Ebola sero/genotypes. In other embodiments, the viral glycoprotein is selected from a species or subspecies of Ebola virus. In some embodiments, the Ebola virus is the Zaire strain of Ebola virus. In certain embodiments, the filovirus glycoprotein is an Ebola virus glycoprotein (GP). In still other embodiments, the filovirus glycoprotein is the Zaire-strain Ebola virus glycoprotein. In certain embodiments, a modified portion of the viral glycoprotein is the mucin-like domain (MLD) of Ebola GP. Without limiting any of the foregoing, all GPs of filoviruses can display heterologous proteins for use in stimulating an immune response against a filovirus (e.g., Ebola) and an antigen of choice, including but not limited to a RBD of SARS-COV-2, SARS-COV-1, MERS, or a mutant thereof.

In embodiments, a modified portion of the viral glycoprotein is a mucin-like domain (MLD) or a mutant thereof.

In some embodiments, the antigen comprises an exogenous antigen, and endogenous antigen, an autoantigen, a neoantigen, a viral antigen, or a tumor antigen. In alternative embodiments, the antigen is an immunogen.

In certain embodiments, the composition elicits, stimulates, induces, promotes, increases or enhances an immune response against both the viral glycoprotein and the antigen. In specific embodiments, the antigen comprises a protein or peptide, or variant, homologue, derivative or subsequence thereof.

In other embodiments, the antigen comprises, consists or consists essentially of a protein or peptide or a variant, homologue, derivative or subsequence thereof from a Coronavirus, or an alphacoronavirus, a betacoronavirus, a gammacoronavirus, or a deltacoronavirus. In non-limiting embodiments, the Coronavirus is one or more of a species or subspecies of Embecovirus, Sarbecovirus, Merbecovirus, Nobevovirus, Hibecovirus, SARS-COV, or MERS-COV. In other embodiments, the Coronavirus is one or more of SARS-COV, SARS-COV-1, SARS-COV-2, MERS-COV, SL-CoV-WIV1, HK84, HKU5, HCoV-OC43, HCoV-HKU1, or HKU9.

In certain embodiments, the compositions described herein further comprise an adjuvant.

As embodied and broadly described herein, the present disclosure is related to method of modulating, eliciting, stimulating, inducing, promoting, increasing, or enhancing an immune response against one or more viral pathogens. In embodiments, the viral pathogen is a Coronavirus, an Ebola virus, or both a Coronavirus (e.g., SARS-COV-2) and an Ebola virus, the method comprising administering the composition or a combination of the compositions described herein, either alone or in combination with any other compound, agent, drug, treatment or any other therapeutic regimen or protocol having a desired therapeutic, beneficial, additive, synergistic or complementary activity or effect in the treatment, prevention, or vaccination against a viral pathogen such as Coronavirus and/or an Ebola virus, or the symptoms or side-effects of infection thereof. In certain embodiments, the method elicits, stimulates, induces, promotes, increases, or enhances an immune response against two or more different species of virus. In certain embodiments, the two or more species are SARS-COV-2 and Ebola virus.

As embodied and broadly described herein, the present disclosure is related to a method of vaccinating against, providing a subject with protection against, or treating a subject for a viral infection, the method comprising administering the composition or a combination of the compositions described herein. In certain embodiments, the method vaccinates against, provides the subject with protection against, or treats a subject for infection with two or more different viral species. In alternative embodiments, the method vaccinates against, provides the subject with protection against, or treats a subject for infection of Ebola virus and Coronavirus, or a species or subspecies thereof, by way of example and not by way of limitation, Zaire Ebola virus and SARS-CoV-2.

As embodied and broadly described herein, the present disclosure is related to a method of preventing, reducing, or inhibiting the sensitization of a subject to or occurrence in the subject of an antibody dependent enhancement of disease or disease upon a secondary or subsequent viral infection or following administration of the composition or combination of the compositions described herein, subsequent to a prior viral infection in the subject or prior to administration to the subject of a vaccine against one or more viruses. In certain embodiments, the viruses are species or subspecies of Ebola virus and Coronavirus, including by way of example and not by way of limitation, Zaire Ebola virus and SARS-COV-2.

As embodied and broadly described herein, the present disclosure is related to a method of formulating a vaccine against a virus (e.g., the Coronavirus and the Ebola virus) that will not elicit, stimulate, induce, promote, increase, enhance or sensitize a subject to an antibody dependent enhancement of disease or infection, the method comprising formulating the vaccine to comprise a composition or a combination of the compositions described herein.

In non-limiting embodiments, the herein described method of inducing, enhancing, or sustaining an immune response against a virus (e.g, Coronavirus and/or Ebola virus) in a subject may afford one to obtain at least one of the following features: reduce virus titer (e.g., reduce Coronavirus or Ebola virus titer), increase or stimulate virus clearance (e.g., increase or stimulate Coronavirus or Ebola virus clearance), reduce or inhibit virus proliferation (e.g., reduce or inhibit Coronavirus or Ebola virus proliferation), reduce or inhibit increases in virus titer or virus proliferation (e.g., reduce or inhibit increases in Ebola virus titer or Coronavirus titer, or reduce or inhibit increases in Ebola virus or Coronavirus proliferation), reduce the amount of virus protein or virus nucleic acid, or reduce or inhibit synthesis of virus protein or virus nucleic acid (e.g., reduce the amount of an Ebola virus or Coronavirus protein or reduce the amount of an Ebola virus or Coronavirus nucleic acid, or reduce or inhibit synthesis of a Coronavirus or an Ebola virus protein or a Coronavirus or an Ebola virus nucleic acid).

In one non-limiting embodiment, the herein described method of inducing, enhancing, or sustaining an immune response against a virus (e.g., Coronavirus and/or an Ebola virus) in a subject includes contacting T cells of a subject with a therapeutically effective amount of a composition of the present disclosure prior to, substantially contemporaneously with or following exposure to or infection of the subject with the same virus (e.g., Coronavirus and/or Ebola virus). For example, contacting T cells of the subject with the effective amount of the composition of the present disclosure may occur within 2-72 hours, 2-48 hours, 4-24 hours, 4-18 hours, or 6-12 hours after a symptom of a viral infection develops.

In a non-limiting embodiment, the antigen is a Coronavirus antigen. In certain embodiments, the Coronavirus is an alphacoronavirus, a betacoronavirus, a gammacoronavirus, or a deltacoronavirus. In certain alternative embodiments, the betacoronavirus is SARS-COV-2.

There herein described method of inducing, enhancing, or sustaining an immune response against a virus in a subject may treat or mitigate symptoms associated with the virus.

In the case where the Coronavirus is SARS-COV-2 or a betacoronavirus, the herein described method of inducing, enhancing, or sustaining an immune response against a Coronavirus in a subject may treat or mitigate symptoms associated with SARS-COV-2 and/or betacoronavirus infection such as, but not limited to, fever, rash, headache, cough, shortness of breath or difficulty breathing, chills (including shaking associated with chills), sore throat, loss of taste or smell, tiredness, difficulty breathing, pain behind the eyes, conjunctivitis, muscle or joint pain, nausea, vomiting, loss of appetite, or secondary infection.

In a non-limiting embodiment, the viral glycoprotein is selected from a filovirus glycoprotein. In certain embodiments, the filovirus is Ebola virus. In certain alternative embodiments, the Ebola virus is the Zaire strain of the Ebola virus.

Adverse symptoms and complications associated with Ebola virus infection and pathology include, for example, e.g., fever, rash, headache, cough, tiredness, difficulty breathing, pain including pain behind the eyes, stomach pain, muscle or joint pain, weakness, fatigue, conjunctivitis, nausea, vomiting, diarrhea, loss of appetite, hemorrhaging, bleeding, bruising, or secondary infection.

In a non-limiting embodiment, the compositions described in the present disclosure include one or more acceptable carriers selected from the acceptable carriers described herein. In embodiments, an acceptable carrier is selected from gold particles, sterile water, saline, glucose, dextrose, or buffered solutions. In embodiments, carriers include auxiliary agents including, but not limited to, diluents, stabilizers (i.e., sugars and amino acids), preservatives, wetting agents, emulsifying agents, pH buffering agents, viscosity enhancing additives, colors and the like.

Additionally, or alternatively, the compositions described in the present disclosure may include one or more pharmaceutically acceptable salts selected from the pharmaceutically acceptable salts described herein. For example, a pharmaceutically acceptable salt may be selected from sodium chloride, potassium chloride, sodium sulfate, ammonium sulfate, or sodium citrate. The concentration of the pharmaceutically acceptable salt can be any suitable concentration known in the art, and may be selected from about 10 mM to about 200 mM.

Additionally, or alternatively, the compositions of the present disclosure may include one or more adjuvant selected from the adjuvants described herein. In different embodiments, an adjuvant can be a naturally occurring adjuvant or a non-naturally occurring adjuvant.

Additionally or alternatively, the compositions described in the present disclosure and/or the methods of the present disclosure may further include one or more components, such as drugs, immunostimulants (such as α-interferon, β-interferon, γ-interferon, granulocyte macrophage colony stimulator factor (GM-CSF), macrophage colony stimulator factor (M-CSF), and interleukin 2 (IL-2), antioxidants, surfactants, flavoring agents, volatile oils, buffering agents, dispersants, propellants, and preservatives.

In an embodiment, the herein described subject can be a mammal, preferably a human.

In an aspect, a composition comprising a modified viral glycoprotein is disclosed, wherein one or more portions of the modified viral glycoprotein are modified to comprise an antigen. In embodiments, the modified viral glycoprotein and the antigen are from different sources. In embodiments, the modified viral glycoprotein comprises a filovirus glycoprotein. In embodiments, the modified viral glycoprotein is a Ebola virus glycoprotein. In embodiments, the one or more portions of the modified viral glycoprotein is a mucin-like domain (MLD). In embodiments, the antigen comprises an exogenous antigen, and endogenous antigen, an autoantigen, a neoantigen, a viral antigen, or a tumor antigen. In embodiments, the antigen is an immunogen. In embodiments, the composition elicits, stimulates, induces, promotes, increases or enhances an immune response against both the modified viral glycoprotein and the antigen. In embodiments, the antigen comprises a protein or peptide, or variant, homologue, derivative or subsequence thereof. In embodiments, the antigen comprises a protein or peptide or a variant, homologue, derivative or subsequence thereof from a Coronavirus. In embodiments, the Coronavirus is selected from a species or subspecies of Embecovirus, Sarbecovirus, Merbecovirus, Nobevovirus, Hibecovirus, SARS-COV, or MERS-COV. In embodiments, the Coronavirus is selected from a species or subspecies of SARS-COV, SARS-COV-2, MERS-COV, SL-COV-WIV1, HK84, HKU5, HCoV-OC43, HCoV-HKU1, or HKU9. In embodiments, the Coronavirus comprises an alphacoronavirus, a betacoronavirus, a gammacoronavirus, or a deltacoronavirus protein or peptide or a variant, homologue, derivative or subsequence thereof. In embodiments, the antigen comprises a SARS-COV-2 protein or peptide or a variant, homologue, derivative or subsequence thereof. In embodiments, the composition further comprises an adjuvant.

In another aspect, a viral vector for encoding the modified viral glycoprotein is disclosed. In embodiments, the viral vector comprises a recombinant vesicular stomatitis virus (rVSV). In other embodiments, the viral vector comprises a recombinant lentiviral vector. In other embodiments, the viral vector comprises a recombinant baculovirus vector, that when expressed to produce baculovirus particles, is capable of being displayed on a baculovirus surface. In other embodiments, the viral vector comprises a recombinant adenovirus or a recombinant baculovirus. In some embodiments, and in respect of the baculovirus vector disclosed herein, the RBD of SARS-COV-2 on the chimeric GP is better able to facilitate the virion uptake by antigen-presenting (APCs) (e.g., lung macrophages and dendritic cells etc.), since ACE2 is universally expressed in different cell types, including APCs. It will be appreciated that antigen uptake by APCs is a crucial step for generating an effective immune response.

In another aspect, a method of modulating an immune response against one or more antigens is disclosed. The method involves administering any one of the compositions disclosed herein. In embodiments, modulating the immune response comprises eliciting, stimulating, inducing, promoting, increasing, or enhancing the immune response against one or more antigens. In embodiments, the composition disclosed herein elicits, stimulates, induces, promotes, increases, or enhances an immune response against a species or subspecies of Coronavirus. In embodiments, the composition disclosed herein elicits, stimulates, induces, promotes, increases, or enhances an immune response against a species or subspecies of Ebola virus. In embodiments, the composition disclosed herein elicits, stimulates, induces, promotes, increases, or enhances an immune response against both a species or subspecies of Coronavirus and a species or subspecies of Ebola virus. In embodiments, the method elicits, stimulates, induces, promotes, increases, or enhances an immune response against a species or subspecies of Coronavirus. In embodiments, the method elicits, stimulates, induces, promotes, increases, or enhances an immune response against a species or subspecies of Ebola virus. In embodiments, the method elicits, stimulates, induces, promotes, increases, or enhances an immune response against both a species or subspecies of Coronavirus and a species or subspecies of Ebola virus.

In another aspect, a method of vaccinating against, providing a subject with protection against, or treating a subject for viral infection is disclosed. The method comprising administering any one or more of the compositions disclosed herein. In embodiments, the method vaccinates against, provides the subject with protection against or treats a subject for infection against two or more different species or subspecies of viruses. In embodiments, the method vaccinates against, provides the subject with protection against or treats a subject for both a species or subspecies of Coronavirus and a species or subspecies of Ebola virus. In embodiments, the method prevents, reduces, or inhibits sensitizing the subject to or occurrence in the subject of an antibody dependent enhancement of disease or disease upon a secondary or subsequent viral infection or following administration of the composition subsequent to a prior viral infection in the subject or prior to administration to the subject of a vaccine against a virus. In embodiments, the viral infection comprises infection from Coronavirus or Ebola virus. In embodiments, the vaccine is comprised to provide the subject protection against Coronavirus or Ebola virus.

In another aspect, a method of formulating a vaccine against a species or subspecies of Ebola virus or Coronavirus that will not elicit, stimulate, induce, promote, increase, enhance or sensitize a subject to an antibody dependent enhancement of disease or infection is provided, the method comprising formulating the vaccine to comprise a composition as disclosed herein.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

In an aspect, a composition is provided comprising a modified viral glycoprotein, wherein one or more portions of the modified viral glycoprotein are modified to comprise an antigen, wherein the antigen comprises a protein or peptide or variant, homologue, derivative or subsequence thereof from a Coronavirus. In embodiments, the modified viral glycoprotein and the antigen are from different sources.

In an aspect, a viral vector is provided comprising any modified glycoprotein described herein. In embodiments, the viral vector comprises a recombinant vesicular stomatitis virus (rVSV), a recombinant lentivirus or a recombinant baculovirus.

In embodiments, the modified viral glycoprotein is an Ebola virus glycoprotein or mutant thereof. In embodiments, the one or more portions of the modified viral glycoprotein is a mucin-like domain (MLD) or mutant thereof.

In embodiments, the antigen comprises an exogenous antigen, and endogenous antigen, an autoantigen, a neoantigen, a viral antigen, or a tumor antigen. In embodiments, the antigen is an immunogen.

In embodiments, any of the compositions described herein elicits, stimulates, induces, promotes, increases or enhances an immune response against both the modified viral glycoprotein and the antigen.

In embodiments, the Coronavirus comprises an alphacoronavirus, a betacoronavirus, a gammacoronavirus, or a deltacoronavirus. In embodiments, the Coronavirus is selected from a species or subspecies of Embecovirus, Sarbecovirus, Merbecovirus, Nobevovirus, Hibecovirus, SARSr-COV, or MERS-COV.

In embodiments, the Coronavirus is selected from a species or subspecies of SARS-CoV, SARS-COV-1, SARS-COV-2, MERS-COV, SL-CoV-WIV1, HK84, HKU5, HCoV-OC43, HCoV-HKU1, or HKU9.

In embodiments, the Coronavirus comprises an alphacoronavirus, a betacoronavirus, a gammacoronavirus, or a deltacoronavirus protein or peptide or a variant, homologue, derivative or subsequence thereof.

In embodiments, the antigen comprises a SARS-COV-2 protein or peptide or a variant, homologue, derivative or subsequence thereof.

In embodiments, any of the compositions described herein, further comprise an adjuvant.

In an aspect, a method of modulating an immune response against one or more antigens is provided, the method comprising administering any of the compositions described herein.

In embodiments, modulating the immune response comprises eliciting, stimulating, inducing, promoting, increasing, or enhancing the immune response against one or more antigens.

In embodiments, any of the compositions described herein elicits, stimulates, induces, promotes, increases, or enhances an immune response against a species or subspecies of Coronavirus. In embodiments, any of the compositions described herein elicits, stimulates, induces, promotes, increases, or enhances an immune response against a species or subspecies of Ebola virus. In embodiments, any of the compositions described herein elicits, stimulates, induces, promotes, increases, or enhances an immune response against both a species or subspecies of Coronavirus and a species or subspecies of Ebola virus. In embodiments, the method elicits, stimulates, induces, promotes, increases, or enhances an immune response against a species or subspecies of Coronavirus. In embodiments, the method elicits, stimulates, induces, promotes, increases, or enhances an immune response against a species or subspecies of Ebola virus. In embodiments, the method elicits, stimulates, induces, promotes, increases, or enhances an immune response against both a species or subspecies of Coronavirus and a species or subspecies of Ebola virus.

In an aspect, a method of vaccinating is provided comprising providing a subject with protection against, or treating a subject for viral infection, the method comprising administering any of the compositions described herein.

In embodiments, the method vaccinates against, provides the subject with protection against or treats a subject for infection against two or more different species or subspecies of viruses. In embodiments, the method vaccinates against, provides the subject with protection against or treats a subject for both a species or subspecies of Coronavirus and a species or subspecies of Ebola virus.

In embodiments, the method prevents, reduces, or inhibits sensitizing the subject to or occurrence in the subject of an antibody dependent enhancement of disease or disease upon a secondary or subsequent viral infection or following administration of any of the compositions described herein subsequent to a prior viral infection in the subject or prior to administration to the subject of a vaccine against a virus.

In embodiments, the viral infection comprises infection from Coronavirus or Ebola virus.

In embodiments, the vaccine is comprised to provide the subject protection against Coronavirus or Ebola virus.

In an aspect, a method is provided of formulating a vaccine against a species or subspecies of Ebola virus or Coronavirus that will not elicit, stimulate, induce, promote, increase, enhance or sensitize a subject to an antibody dependent enhancement of disease or infection, the method comprising formulating the vaccine to comprise any of the compositions described herein.

### DETAILED DESCRIPTION

A detailed description of one or more embodiments of the invention is provided below along with accompanying figures that illustrate the principles of the invention. The invention is described in connection with such embodiments, but the invention is not limited to any embodiment. The scope of the invention is limited only by the claims. Numerous specific details are set forth in the following description in order to provide a thorough understanding of the invention. These details are provided for the purpose of non-limiting examples and the invention may be practiced according to the claims without some or all of these specific details. For the purpose of clarity, technical material that is known in the technical fields related to the invention has not been described in detail so that the invention is not unnecessarily obscured.

### Overview of Disclosure

The present disclosure describes experimental results and line of reasoning which supports the development of more effective viral vaccines (e.g., Coronavirus and/or Ebola virus vaccines) and/or treatment approaches, than what has been previously described. As described herein, and in certain embodiments, the vaccine and/or treatment approach relates to Ebola virus. As described herein, and in certain embodiments, the vaccine and/or treatment approach relates to Coronavirus. As described herein, and in certain embodiments, the vaccine and/or treatment approach relates to both Ebola virus and Coronavirus. In particular embodiments, the Coronavirus is SARS-COV-2. As described herein, and in certain embodiments, the vaccine and/or treatment approach relates to the use of a composition that comprises a modified viral glycoprotein in which one or more portions of the modified glycoprotein are modified to comprise an antigen.

### Definitions and Interpretation

Unless otherwise defined, all technical and scientific terms used herein have the same meaning as commonly understood by a person of ordinary skill in the art to which the present invention pertains. As used herein, and unless stated otherwise or required otherwise by context, each of the following terms shall have the definition set forth below.

If and as used herein, the term “administering”, when used in relation to an expression vector, nucleic acid molecule, or a delivery vehicle (such as a chitosan nanoparticle) to a cell, refers to transducing, transfecting, electroporation, translocating, fusing, phagocytosing, shooting or ballistic methods, etc., i.e., any means by which a protein or nucleic acid can be transported across a cell membrane and preferably into the nucleus of a cell.

If and as used herein, the term “antibody (Ab) dependent enhancement of infection (ADE)” refers to a phenomenon in which a subject who has antibodies against a virus (e.g., coronavirus), due to a previous viral (e.g., Coronavirus, Ebola virus, Dengue virus) infection or exposure to viral antigen (e.g., vaccination, immunization, receipt of maternal anti-viral antibodies, etc.), suffers from enhanced or a more severe illness after a secondary or subsequent infection with a virus, or after a viral vaccination or immunization. Typically, the more severe symptoms include one or more of hemorrhagic fever/shock syndrome, increased viral load, increased vascular permeability, increased hemorrhagic manifestations, thrombocytopenia, and shock, compared to the acute self-limited illness typically caused by viral infections in subjects who have not been vaccinated, immunized or previously infected with said virus. Although not wishing to be bound by any theory, ADE is believed to be a consequence of the presence of serotype cross-reactive antibodies enhancing viral infection of cells resulting in higher viral loads and a more severe illness upon subsequent exposure or infection of the subject to a viral antigen. Methods and uses of the invention therefore include methods and uses that do not substantially or detectably cause, elicit or stimulate one or more symptoms characteristic of ADE, or more broadly ADE, in a subject.

In addition to ADE, there may be other adverse symptoms that result from, or be enhanced or more severe, when a subject who has antibodies against a virus (e.g., Coronavirus or Ebola virus) (e.g., due to a prior infection, exposure, vaccination, immunization, maternal antibodies etc.) becomes infected with a virus (e.g., a Coronavirus or Ebola virus), or receives a related viral vaccination (e.g., a Coronavirus or Ebola vaccination or immunization), as compared to a subject that has not been vaccinated, immunized or previously infected with the same viral strain or subspecies (e.g., a Coronavirus or Ebola virus strain or subspecies). Such adverse symptoms that may result from, or may be enhanced or more severe include, for example, fever, headache, rash, liver damage, diarrhea, nausea, vomiting or abdominal pain. It is intended that the methods and uses of the invention therefore also include methods and uses that do not substantially elicit, enhance or worsen one or more such other adverse symptoms that may be elicited, enhanced or be more severe in a subject who has antibodies against the virus (e.g., a Coronavirus or Ebola virus) as compared to a subject that does not have antibodies against the same virus (e.g., a Coronavirus or Ebola virus).

If and as used herein, the term “recombinant”, when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed or not expressed at all.

If and as used herein, the term “nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. The term nucleic acid is used interchangeably with gene, cDNA, mRNA, oligonucleotide, and polynucleotide. The nucleotide sequences are displayed herein in the conventional 5′-3′ orientation.

If and as used herein, the terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins. The polypeptide sequences are displayed herein in the conventional N-terminal to C-terminal orientation.

If and used herein, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, carboxyglutamate, and O-phosphoserine. The expression “amino acid analogs” refers to compounds that have the same basic chemical structure as a naturally occurring amino acid, i.e., an .alpha. carbon that is bound to a hydrogen, a carboxyl group, an amino group, and an R group, e.g., homoserine, norleucine, methionine sulfoxide, methionine, and methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

If and used herein, the term “conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon in an amino acid herein, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid and nucleic acid sequences, individual substitutions, deletions or additions that alter, add or delete a single amino acid or-nucleotide or a small percentage of amino acids or nucleotides in the sequence create a “conservatively modified variant,” where the alteration results in the substitution of an amino acid with a chemically similar amino acid. Conservative substitution tables providing functionally similar amino acids are well known in the art.

For example, the following groups each contain amino acids that are conservative substitutions for one another (see, e.g., Creighton, Proteins (1984) W. H. Freeman, New York, pages 6-20, for a discussion of amino acid properties):

Alanine (A), Glycine (G)
Serine (S), Threonine (T)
Aspartic acid (D), Glutamic acid (E)
Asparagine (N), Glutamine (Q)
Cysteine (C), Methionine (M)
Arginine (E), Lysine (K), Histidine (H)
Isoleucine (I), Leucine (L), Valine (V)
Phenylalanine (F), Tyrosine (Y), Tryptophan (W)

In light of the present disclosure, in particular in view of the experimental data described in the examples of the present text, the person of skill will readily understand which amino acid may be substituted, deleted or added to a given sequence to create a conservatively modified variant comprising an amino acid sequence which is at least at least 95%, or at least 96%, or at least 97%, or at least 98%, or at least 99%, identical to one or more amino acid sequence set forth in the table above without undue effort.

If and used herein, the term “primers” are isolated nucleic acids that are annealed to a complementary target DNA strand by nucleic acid hybridization to form a hybrid between the primer and the target DNA strand, then extended along the target DNA strand by a polymerase, e.g., a DNA polymerase. Primer pairs of the present invention refer to their use for amplification of a target nucleic acid sequence, e.g., by the polymerase chain reaction (PCR) or other conventional nucleic-acid amplification methods, such as qPCR.

If and used herein, the phrases “coding sequence,” “structural sequence,” and “structural nucleic acid sequence” refer to a physical structure comprising an orderly arrangement of nucleic acids. The nucleic acids are arranged in a series of nucleic acid triplets that each form a codon. Each codon encodes for a specific amino acid. Thus, the coding sequence, structural sequence, and structural nucleic acid sequence encode a series of amino acids forming a protein, polypeptide, or peptide sequence. The coding sequence, structural sequence, and structural nucleic acid sequence may be contained within a larger nucleic acid molecule, vector, or the like. In addition, the orderly arrangement of nucleic acids in these sequences may be depicted in the form of a sequence listing, figure, table, electronic medium, or the like.

If and used herein, the phrases “DNA sequence,” “nucleic acid sequence,” and “nucleic acid molecule” refer to a physical structure comprising an orderly arrangement of nucleic acids. The DNA sequence or nucleic acid sequence may be contained within a larger nucleic acid molecule, vector, or the like. In addition, the orderly arrangement of nucleic acids in these sequences may be depicted in the form of a sequence listing, figure, table, electronic medium, or the like.

If and used herein, the term “expression” refers to the transcription of a gene to produce the corresponding mRNA and translation of this mRNA to produce the corresponding gene product (i.e., a peptide, polypeptide, or protein).

If and used herein, the term “isolated” refers to material, such as a nucleic acid or a protein, which is: (1) substantially or essentially free from components which normally accompany or interact with the material as found in its naturally occurring environment or (2) if the material is in its natural environment, the material has been altered by deliberate human intervention to a composition and/or placed at a locus in the cell other than the locus native to the material.

If and used herein, the terms “treating” or “treatment” refers to a process by which an infection or a disease or the symptoms of an infection or a disease associated with a viral strain (e.g., a Coronavirus and/or Ebola virus strain) are prevented, alleviated or completely eliminated. As used herein, the term “prevented” or “preventing” refers to a process by which an infection or a disease or symptoms of an infection or a disease associated with Coronavirus and/or Ebola virus are obstructed or delayed.

If and as used herein, a “sufficient amount” or “effective amount” or an “amount sufficient” or an “amount effective” refers to an amount that provides, in single (e.g., primary) or multiple (e.g., booster) doses, alone or in combination with one or more other compounds, treatments, therapeutic regimens or agents (e.g., a drug), a long term or a short term detectable or measurable improvement in a given subject or any objective or subjective benefit to a given subject of any degree or for any time period or duration (e.g., for minutes, hours, days, months, years, or cured).

An amount sufficient or an amount effective can but need not be provided in a single administration and can but need not be achieved by administration of a viral protein, peptide, or a variant, modification, homologue, derivative or subsequence thereof (e.g., a Coronavirus or Ebola virus protein, peptide, or a variant, modification, homologue, derivative or sequence thereof) alone or in a combination composition or method that includes a second active. In addition, an amount sufficient or an amount effective need not be sufficient or effective if given in single or multiple doses without a second or additional administration or dosage, since additional doses, amounts or duration above and beyond such doses, or additional antigens, compounds, drugs, agents, treatment or therapeutic regimens may be included in order to provide a given subject with a detectable or measurable improvement or benefit to the subject. For example, to increase, enhance, improve or optimize immunization and/or vaccination, after an initial or primary administration of one or more viral proteins peptides, or variants, modifications, homologues, derivatives or subsequences thereof (e.g., a Coronavirus or Ebola virus protein, peptide, or a variant, modification, homologue, derivative or sequence thereof) to a subject, the subject can be administered one or more additional “boosters” of one or more viral peptides, subsequences, portions or modifications thereof (e.g., Coronavirus or Ebola virus peptides, subsequences, portions or modifications thereof). Such subsequent “booster” administrations can be of the same or a different formulation, dose or concentration, route, etc.

An amount sufficient or an amount effective need not be therapeutically or prophylactically effective in each and every subject treated, nor a majority of subjects treated in a given group or population. An amount sufficient or an amount effective means sufficiency or effectiveness in a particular subject, not a group of subjects or the general population. As is typical for such methods, different subjects will exhibit varied responses to treatment.

If and used herein, the expression “an acceptable carrier” refers to a vehicle for containing a compound that can be administered to a subject without significant adverse effects.

As used herein, the term “adjuvant” means a substance added to the composition of the invention to increase the composition's immunogenicity. The mechanism of how an adjuvant operates is not entirely known. Some adjuvants are believed to enhance the immune response (humoral and/or cellular response) by slowly releasing the antigen, while other adjuvants are strongly immunogenic in their own right and are believed to function synergistically.

With respect to the present disclosure, an adjuvant may be selected from aluminum hydroxide or mineral oil, and a stimulator of immune responses, such as Bordetella pertussis or Mycobacterium tuberculosis derived proteins. Suitable adjuvants are commercially available as, for example, Freund's Incomplete Adjuvant and Complete Adjuvant (Pifco Laboratories, Detroit, Mich.); Merck Adjuvant 65 (Merck and Company, Inc., Rahway, N.J.); aluminum salts such as aluminum hydroxide gel (alum) or aluminum phosphate; salts of calcium, iron or zinc; an insoluble suspension of acylated tyrosine acylated sugars; cationically or anionically derivatized polysaccharides; polyphosphazenes; biodegradable microspheres; and Quil A. Suitable adjuvants also include, but are not limited to, toll-like receptor (TLR) agonists, particularly toll-like receptor type 4 (TLR-4) agonists (e.g., monophosphoryl lipid A (MPL), synthetic lipid A, lipid A mimetics or analogs), aluminum salts, cytokines, saponins, muramyl dipeptide (MDP) derivatives, CpG oligos, lipopolysaccharide (LPS) of gram-negative bacteria, polyphosphazenes, emulsions, virosomes, cochleates, poly(lactide-co-glycolides) (PLG) microparticles, poloxamer particles, microparticles, liposomes, oil-in-water emulsions, MF59, and squalene. In some embodiments, the adjuvants are not bacterially-derived exotoxins. In an embodiment, adjuvants may include adjuvants which stimulate a Th1 type response such as 3DMPL or QS21. Adjuvants may also include certain synthetic polymers such as poly amino acids and co-polymers of amino acids, saponin, paraffin oil, and muramyl dipeptide. Adjuvants also encompass genetic adjuvants such as immunomodulatory molecules encoded in a co-inoculated DNA, or as CpG oligonucleotides. The co-inoculated DNA can be in the same plasmid construct as the plasmid immunogen or in a separate DNA vector. The reader can refer to Vaccines (Basel). 2015 June; 3(2): 320-343 for further examples of suitable adjuvants.

If and used herein, the expression “ELISPOT” refers to the known Enzyme-Linked ImmunoSpot assay which typically allows visualization of the secretory product(s) of individual activated or responding cells. Each spot that develops in the assay represents a single reactive cell. Thus, the ELISPOT assay provides both qualitative (regarding the specific cytokine or other secreted immune molecule) and quantitative (the frequency of responding cells within the test population) information. Generally speaking, in an ELISPOT assay, the membrane surfaces in a 96-well PVDF-membrane microtiter plate are coated with capture antibody that binds a specific epitope of the cytokine being assayed. During the cell incubation and stimulation step, a biological sample (typically containing PBMCs) is seeded into the wells of the plate along with the antigen (which can be a peptide as described in the present disclosure), and forms a monolayer on the membrane surface of the well. As the antigen-specific cells are activated, they release the cytokine, which is captured directly on the membrane surface by the immobilized antibody. The cytokine is thus “captured” in the area directly surrounding the secreting cell, before it has a chance to diffuse into the culture media, or to be degraded by proteases and bound by receptors on bystander cells. Subsequent detection steps visualize the immobilized cytokine as an ImmunoSpot; essentially the secretory footprint of the activated cell.

If and used herein, the terms “determining,” “measuring,” “evaluating,” “assessing,” and “assaying” generally refer to any form of measurement, and include determining if an element is present or not in a biological sample. These terms include both quantitative and/or qualitative determinations, which both require sample processing and transformation steps of the biological sample. Assessing may be relative or absolute. The phrase “assessing the presence of” can include determining the amount of something present, as well as determining whether it is present or absent.

If and as used herein, the expression “biological sample” includes, in the present disclosure, any biological sample that is suspected of comprising a T cell, such as for example but without being limited thereto, blood and fractions thereof, urine, excreta, semen, seminal fluid, seminal plasma, prostatic fluid, pre-ejaculatory fluid (Cowper's fluid), pleural effusion, tears, saliva, sputum, sweat, biopsy, ascites, amniotic fluid, lymph, vaginal secretions, endometrial secretions, gastrointestinal secretions, bronchial secretions, breast secretions, and the like. In one non-limiting embodiment, a herein described biological sample can be obtained by any known technique, for example by drawing, by non-invasive techniques, or from sample collections or banks, etc.

If and as used herein, the expression “treatment” includes inducing, enhancing, or sustaining an immune response against a Coronavirus or Ebola virus infection or symptoms associated thereto. For example, the treatment may induce, increase, promote or stimulate anti-Coronavirus or anti-Ebola virus activity of immune system cells in a subject following the treatment. For example, the immune system cells may include T cells, including CD4⁺ T cells, CD8⁺ T cells, and/or B cells.

If and as used herein, the expression “therapeutically effective amount” may include the amount necessary to allow the component or composition to which it refers to perform its immunological role without causing overly negative effects in the host to which the component or composition is administered. The exact amount of the components to be used or the composition to be administered will vary according to factors such as the type of condition being treated, the type and age of the subject to be treated, the mode of administration, as well as the other ingredients in the composition.

Other examples of implementations will become apparent to the reader in view of the teachings of the present description and as such, will not be further described here.

Note that titles or subtitles may be used throughout the present disclosure for convenience of a reader, but in no way these should limit the scope of the invention. Moreover, certain theories may be proposed and disclosed herein; however, in no way they, whether they are right or wrong, should limit the scope of the invention so long as the invention is practiced according to the present disclosure without regard for any particular theory or scheme of action.

All references cited throughout the specification are hereby incorporated by reference in their entirety for all purposes.

It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions will control.

As used in the present disclosure, the terms “around”, “about” or “approximately” shall generally mean within the error margin generally accepted in the art. Hence, numerical quantities given herein generally include such error margin such that the terms “around”, “about” or “approximately” can be inferred if not expressly stated.

### Detailed Description of Aspects and Embodiments of the Disclosure

In accordance with the invention, treatment methods are provided that include therapeutic (following infection) and prophylactic (prior to viral exposure, infection or pathology) methods. For example, therapeutic and prophylactic methods of treating a subject for a viral infection (e.g., a Coronavirus and/or Ebola virus infection) include treatment of a subject having or at risk of having a viral infection or pathology (e.g., a Coronavirus and/or Ebola virus infection or pathology), treating a subject with a viral infection (e.g., a Coronavirus and/or Ebola virus infection), and methods of protecting a subject from a viral infection (e.g., a Coronavirus and/or Ebola virus infection) (e.g., provide the subject with protection against Coronavirus and/or Ebola virus infection), to decrease or reduce the probability of a viral infection (e.g., a Coronavirus and/or Ebola virus infection) in a subject, to decrease or reduce susceptibility of a subject to a viral infection (e.g., a Coronavirus and/or Ebola virus infection), or to inhibit or prevent a viral infection (e.g., a Coronavirus and/or Ebola virus infection) in a subject, and to decrease, reduce, inhibit or suppress transmission of a virus (e.g., the Coronavirus and/or Ebola virus) from a host to a subject.

Such methods include administering viral proteins, peptides, or variants, modifications, homologues, derivatives or subsequences thereof (e.g., Coronavirus and/or Ebola virus proteins, peptides, or variants, modifications, homologues, derivatives or subsequences thereof) to therapeutically or prophylactically treat (vaccinate or immunize) a subject having or at risk of having a viral infection (e.g., a Coronavirus and/or Ebola virus infection or pathology). Accordingly, methods can treat a viral infection or pathology (e.g., the Coronavirus and/or Ebola virus infection or pathology), or provide the subject with protection from infection (e.g., prophylactic protection).

In an embodiment, a method includes administering to a subject an amount of a viral protein, peptide, or a variant, modification, homologue, derivative or subsequence thereof (e.g., a Coronavirus and/or Ebola virus protein, peptide, or a variant, modification, homologue, derivative or subsequence thereof) sufficient to treat the subject for the Coronavirus and/or Ebola virus infection or pathology, or one or more physiological conditions, disorders, illness, diseases or symptoms caused by or associated with the virus infection or pathology.

Viral proteins, peptides, or variants, modifications, homologues, derivatives or subsequences thereof (e.g., Coronavirus and Ebola virus proteins, peptides, or variants, modifications, homologues, derivatives or subsequences thereof) may include B cell epitopes and/or T cell epitopes. In one embodiment, a method includes administering an amount of viral protein, peptide, or a variant, modification, homologue, derivative or subsequence thereof (e.g., a Coronavirus and/or Ebola virus protein, peptide, or a variant, modification, homologue, derivative or subsequence thereof) (e.g., B cell and/or T cell epitope(s)) to a subject in need thereof, sufficient to provide the subject with protection against a virus infection or pathology (e.g., a Coronavirus or Ebola virus infection or pathology). In another embodiment, a method includes administering an amount of a viral protein, peptide, or variant, modification, homologue, derivative or subsequence thereof (e.g., a Coronavirus and/or Ebola virus protein, peptide, or a variant, modification, homologue, derivative or subsequence thereof) (e.g., B cell epitope(s) and/or T cell epitope(s)) to a subject in need thereof sufficient to treat, vaccinate or immunize the subject against a virus infection or pathology (e.g., the Coronavirus and/or Ebola virus infection or pathology).

In accordance with the invention, methods of inducing, increasing, promoting or stimulating anti-viral activity (e.g., anti-Coronavirus and/or anti-Ebola virus activity) of CD8⁺ T cells or CD4⁺ T cells in a subject are provided. In one embodiment, a method includes administering to a subject an amount of a viral T cell epitope (e.g., a Coronavirus and/or Ebola virus T cell epitope) sufficient to induce, increase, promote or stimulate anti-viral activity (e.g., anti-Coronavirus and/or anti-Ebola virus activity) of CD8⁺ T cells or CD4⁺ T cells in the subject. In accordance with the invention, methods of inducing, increasing, promoting or

stimulating anti-viral activity (e.g., anti-Coronavirus and/or anti-Ebola virus activity) of B cells in a subject are provided. In one embodiment, a method includes administering to a subject an amount of a viral (e.g., a Coronavirus and/or Ebola virus) B cell epitope sufficient to induce, increase, promote or stimulate anti-viral (e.g., anti-Coronavirus and/or anti-Ebola virus) activity of B cells in the subject.

In methods of the present disclosure, any appropriate Coronavirus and/or Ebola protein, peptide, or a variant, modification, homologue, derivative or subsequence thereof can be administered. Non-limiting examples include Coronavirus and/or Ebola virus peptides, subsequences, portions or modifications thereof of an Ebola virus or Coronavirus (e.g., SARS-CoV-2). Additional non-limiting examples include an Ebola virus glycoprotein, and/or a Coronavirus or SARS-COV-2 virus protein (e.g., spike (S), membrane (M) or nucleoprotein (N)) T cell epitope or B cell epitope, such as a subsequence, portion or modification of a sequence in such proteins. In some embodiments, the subsequence of the Coronavirus or SARS-COV-2 virus spike protein is the receptor binding domain (RBD) of said spike protein. In other embodiments, the subsequence is the RBD of SARS-COV-1 or MERS-COV.

In particular embodiments, one or more disorders, diseases, physiological conditions, pathologies and symptoms associated with or caused by a viral infection or pathology (e.g., a Coronavirus or Ebola virus infection or pathology) will respond to treatment. In particular methods embodiments, treatment methods reduce, decrease, suppress, limit, control or inhibit viral numbers or titers (e.g., Coronavirus and/or Ebola virus numbers or titer); reduce, decrease, suppress, limit, control or inhibit pathogen proliferation or replication; reduce, decrease, suppress, limit, control or inhibit the amount of a pathogen protein; or reduce, decrease, suppress, limit, control or inhibit the amount of a viral nucleic acid (e.g., a Coronavirus and/or Ebola virus nucleic acid). In additional particular methods embodiments, treatment methods include an amount of a viral peptide, subsequence or portion thereof (e.g., a Coronavirus and/or Ebola virus peptide, subsequence or portion thereof) sufficient to increase, induce, enhance, augment, promote or stimulate an immune response against a virus (e.g., a Coronavirus and/or Ebola virus); increase, induce, enhance, augment, promote or stimulate virus clearance or removal (e.g., Coronavirus and/or Ebola virus clearance or removal); or decrease, reduce, inhibit, suppress, prevent, control, or limit transmission of a virus (e.g., Coronavirus and/or Ebola virus) to a subject (e.g., transmission from a host to a subject). In further particular methods embodiments, treatment methods include an amount of viral peptide, subsequence or portion thereof (e.g., a Coronavirus and/or Ebola peptide, subsequence or portion thereof) sufficient to protect a subject from a viral infection or pathology (e.g., a Coronavirus and/or Ebola virus infection or pathology), or reduce, decrease, limit, control or inhibit susceptibility to a viral infection or pathology (e.g., a Coronavirus and/or Ebola virus infection or pathology).

Methods of the present disclosure include treatment methods, which result in any therapeutic or beneficial effect. In various methods embodiments, viral infection, proliferation, or pathogenesis (e.g., Coronavirus and/or Ebola virus infection, proliferation or pathogenesis) is reduced, decreased, inhibited, limited, delayed or prevented, or a method decreases, reduces, inhibits, suppresses, prevents, controls or limits one or more adverse (e.g., physical) symptoms, disorders, illnesses, diseases or complications caused by or associated with a particular viral infection, proliferation or replication, or pathology (e.g., a Coronavirus and/or Ebola virus infection, proliferation or replication, or pathology) (see herein). In additional various particular embodiments, treatment methods include reducing, decreasing, inhibiting, delaying or preventing onset, progression, frequency, duration, severity, probability or susceptibility of one or more adverse symptoms, disorders, illnesses, diseases or complications caused by or associated with a particular viral infection, proliferation or replication, or pathology (e.g., a Coronavirus and or Ebola virus infection, proliferation or replication, or pathology) (see herein). In further various particular embodiments, treatment methods include improving, accelerating, facilitating, enhancing, augmenting, or hastening recovery of a subject from a particular viral infection or pathogenesis (e.g., a Coronavirus and/or Ebola virus infection or pathogenesis), or one or more adverse symptoms, disorders, illnesses, diseases or complications caused by or associated with a viral infection, proliferation or replication, or pathology (e.g., a Coronavirus and/or Ebola virus infection, proliferation or replication, or pathology) (see herein). In yet additional various embodiments, treatment methods include stabilizing infection, proliferation, replication, pathogenesis, or an adverse symptom, disorder, illness, disease or complication caused by or associated with a viral infection, proliferation or replication, or pathology (e.g., a Coronavirus and/or Ebola virus infection, proliferation or replication, or pathology), or decreasing, reducing, inhibiting, suppressing, limiting or controlling transmission of a virus (e.g., a Coronavirus and/or Ebola virus) from a to an uninfected subject.

A therapeutic or beneficial effect of treatment is therefore any objective or subjective measurable or detectable improvement or benefit provided to a particular subject. A therapeutic or beneficial effect can but need not be complete ablation of all or any particular adverse symptom, disorder, illness, disease or complication caused by or associated with a viral infection, proliferation or replication, or pathology (e.g., a Coronavirus and/or Ebola virus infection, proliferation or replication, or pathology) (see herein). Thus, a satisfactory clinical endpoint is achieved when there is an incremental improvement or a partial reduction in an adverse symptom, disorder, illness, disease or complication caused by or associated with a viral infection, proliferation or replication, or pathology (e.g., a Coronavirus and/or Ebola virus infection, proliferation or replication, or pathology), or an inhibition, decrease, reduction, suppression, prevention, limit or control of worsening or progression of one or more adverse symptoms, disorders, illnesses, diseases or complications caused by or associated with a viral infection, viral numbers, titers, proliferation or replication, viral protein or nucleic acid, or a viral pathology, over a short or long duration (hours, days, weeks, months, etc.) (e.g., a Coronavirus and/or Ebola virus infection, Coronavirus and/or Ebola virus numbers, titers, proliferation or replication, Coronavirus or Ebola virus protein or nucleic acid, or Coronavirus and/or Ebola virus pathology, over a short or long duration (hours, days, weeks, months, etc.)).

A therapeutic or beneficial effect also includes reducing or eliminating the need, dosage frequency or amount of a second active such as another drug or other agent (e.g., anti-viral) used for treating a subject having or at risk of having a viral infection or pathology (e.g., a Coronavirus and/or Ebola virus infection or pathology). For example, reducing an amount of an adjunct therapy, for example, a reduction or decrease of a treatment for a viral infection or pathology (e.g., a Coronavirus or Ebola virus infection or pathology), or a vaccination or immunization protocol is considered a beneficial effect. In addition, reducing or decreasing an amount of a viral antigen (e.g., a Coronavirus or Ebola virus antigen) used for vaccination or immunization of a subject to provide protection to the subject is considered a beneficial effect.

Adverse symptoms and complications associated with viral infection and pathology (e.g., Coronavirus infection and pathology) include, but are not limited to, for example, e.g., fever, rash, headache, cough, shortness of breath or difficulty breathing, chills (including shaking associated with chills), sore throat, loss of taste or smell, tiredness, difficulty breathing, pain behind the eyes, conjunctivitis, muscle or joint pain, nausea, vomiting, loss of appetite, or secondary infection. Other symptoms of viral infections or pathogenesis such as Coronavirus infection or pathogenesis, are known to one of skill in the art and treatment thereof in accordance with the invention is provided. Thus, the aforementioned symptoms and complications are treatable in accordance with the invention.

Adverse symptoms and complications associated with Ebola virus infection and pathology include, for example, e.g., fever, rash, headache, cough, tiredness, difficulty breathing, pain including pain behind the eyes, stomach pain, muscle or joint pain, weakness, fatigue, conjunctivitis, nausea, vomiting, diarrhea, loss of appetite, hemorrhaging, bleeding, bruising, or secondary infection. Other symptoms of Coronavirus and Ebola virus infection or pathogenesis are known to one of skill in the art and treatment thereof in accordance with the invention is provided. Thus, the aforementioned symptoms and complications are treatable in accordance with the invention.

Methods and compositions of the invention also include increasing, stimulating, promoting, enhancing, inducing or augmenting an anti-viral B cell, CD4⁺ and/or CD8⁺ T cell responses (e.g., an anti-Coronavirus, anti-Ebola virus, and/or anti-SARS-COV-2 B cell, CD4⁺ and/or CD8⁺ T cell responses) in a subject, such as a subject with or at risk of viral infection or pathology (e.g., a Coronavirus, Ebola virus, and/or SARS-COV-2 virus infection or pathology). In one embodiment, a method includes administering to a subject an amount of a viral protein, peptide, or a variant, modification, homologue, derivative or subsequence thereof (e.g., a Coronavirus and/or Ebola virus protein, peptide, or a variant, modification, homologue, derivative or subsequence thereof) sufficient to increase, stimulate, promote, enhance, augment or induce an anti-viral B cell CD4⁺ and/or CD8⁺ T cell response (e.g., an anti-Coronavirus, anti-Ebola virus, and/or anti-SARS-COV-2 B cell, CD4⁺ and/or CD8⁺ T cell response) in the subject. In another embodiment, a method includes administering to a subject an amount of a viral protein, peptide, or a variant, modification, homologue, derivative or subsequence thereof and administering a viral antigen, live or attenuated virus, or a nucleic acid encoding all or a portion (e.g., a B cell or T cell epitope) of any protein or proteinaceous viral antigen (e.g., a Coronavirus and/or Ebola virus protein, peptide, or a variant, modification, homologue, derivative or subsequence thereof and administering a Coronavirus and/or Ebola virus antigen, live or attenuated Ebola or Coronavirus, or a nucleic acid encoding all or a portion (e.g., a B cell or T cell epitope) of any protein or proteinaceous Coronavirus or Ebola virus antigen) sufficient to increase, stimulate, promote, enhance, augment or induce anti-viral B cell, CD4⁺ T cell and/or CD8⁺ T cell responses (e.g., anti-Coronavirus and/or anti-Ebola B cell, CD4⁺ T cell and/or CD8⁺ T cell response) in the subject.

Methods of the present disclosure additionally include, among other things, increasing production of a Th1 cytokine (e.g., IFN-gamma, TNF-alpha, IL-1alpha, IL-2, IL-6, IL-8, etc.) or other signaling molecule (e.g., CD40L) in vitro or in vivo. In an embodiment, a method includes administering to a subject in need thereof an amount of a viral protein, peptide, or variant, modification, homologue, derivative or subsequence thereof (e.g., a Coronavirus and/or Ebola virus protein, peptide, or a variant, modification, homologue, derivative or subsequence thereof) sufficient to increase production of a Th1 cytokine in the subject (e.g., IFN-gamma, TNF-alpha, IL-1alpha, IL-2, IL-6, IL-8, etc.) or other signaling molecule (e.g., CD40L).

Methods of the present disclosure additionally include, among other things, decreasing production of a Th1 cytokine (e.g., IFN-gamma, TNF-alpha, IL-1alpha, IL-2, IL-6, IL-8, etc.) or other signaling molecule (e.g., CD40L) in vitro or in vivo where viral infection (e.g., a Coronavirus or Ebola virus infection) has become severe and a subject is suffering from an adverse immune response. In one embodiment, a method includes administering to a subject in need thereof a composition sufficient to decrease production of a Th1 cytokine in the subject (e.g., IFN-gamma, TNF-alpha, IL-1alpha, IL-2, IL-6, IL-8, etc.) or other signaling molecule (e.g., CD40L).

Methods, uses and compositions of the present disclosure include administration of a viral protein, peptide, or variant, modification, homologue, derivative or subsequence thereof (e.g., a Coronavirus and/or Ebola virus protein, peptide, or a variant, modification, homologue, derivative or subsequence thereof) to a subject prior to contact, exposure or infection by a particular virus (e.g., a Coronavirus such as SARS-COV-2, Ebola virus, etc.), administration prior to, substantially contemporaneously with or after a subject has been contacted by, exposed to or infected with a particular virus (e.g., a Coronavirus or Ebola virus such as SARS-COV-2, Ebola virus, etc.), and administration prior to, substantially contemporaneously with or after a particular virus (e.g., a Coronavirus or Ebola virus such as SARS-COV-2, Ebola virus, etc.) pathology or development of one or more adverse symptoms, disorders, illness or diseases caused by or associated with a particular viral infection or pathology (e.g., a Coronavirus or Ebola virus infection or pathology). A subject infected with a particular virus (e.g., a Coronavirus or Ebola virus) may have an infection over a period of 1-5, 5-10, 10-20, 20-30, 30-50, 50-100 hours, days, months, or years.

Compositions (e.g., Coronavirus and/or Ebola virus proteins or peptides, or a variant, modification, homologue, derivative or subsequence thereof) and uses and methods of the present disclosure can be combined with any compound, agent, drug, treatment or other therapeutic regimen or protocol having a desired therapeutic, beneficial, additive, synergistic or complementary activity or effect. Exemplary combination compositions and treatments include multiple T cell epitopes as set for the herein, second actives, such as anti-viral compounds, agents, and drugs (e.g., anti-Coronavirus or Ebola-virus compounds, agents and drugs), as well as agents that assist, promote, stimulate or enhance efficacy. Such anti-viral drugs, agents, treatments, and therapies (e.g., anti-Coronavirus or anti-Ebola virus drugs, agents, treatments and therapies) can be administered or performed prior to, substantially contemporaneously with or following any other method of the invention, for example, a therapeutic method of treating a subject for a particular virus infection or pathology (e.g., a Coronavirus or Ebola virus infection or pathology), or a method of prophylactic treatment of a subject for a particular viral infection (e.g., a Coronavirus or Ebola virus infection).

Viral proteins, peptides, or variants, modifications, homologues, derivatives or subsequences thereof (e.g., Coronavirus and/or Ebola virus proteins, peptides, or variants, modifications, homologues, derivatives or subsequences thereof) can be administered as a combination composition, or administered separately, such as concurrently or in series or sequentially (prior to or following) administering a second active, to a subject. The invention therefore provides combinations in which a method or use of the invention is used in a combination with any compound, agent, drug, therapeutic regimen, treatment protocol, process, remedy or composition, such as an anti-viral (e.g., anti-Ebola virus or anti-Coronavirus) or immune stimulating, enhancing or augmenting protocol, or pathogen vaccination or immunization (e.g., prophylaxis) set forth herein or known in the art. The compound, agent, drug, therapeutic regimen, treatment protocol, process, remedy or composition can be administered or performed prior to, substantially contemporaneously with or following administration of one or more viral proteins, or variants, modifications, homologues, derivatives or subsequences thereof (e.g., Coronavirus or Ebola-virus proteins, peptides, or variants, modifications, homologues, derivatives or subsequences thereof), or a nucleic acid encoding all or a portion of a viral protein, peptide, or a variant, modification, homologue, derivative or subsequence thereof (e.g., a Coronavirus or Ebola-virus protein, peptide, or a variant, modification, homologue, derivative or subsequence thereof), to a subject. Specific non-limiting examples of combination embodiments therefore include the foregoing or other compound, agent, drug, therapeutic regimen, treatment protocol, process, remedy or composition.

An exemplary combination is a Coronavirus protein, peptide, variant, modification, homologue, derivative or subsequence thereof (e.g., a B cell, CD4⁺ T cell, or CD8⁺ T cell epitope), for example but not by way of limitation, the receptor-binding domain of the SARS-COV-2 spike glycoprotein, bound to a glycoprotein from the family Filoviridae, for example, the Ebola virus glycoprotein, wherein the Coronavirus protein, peptide, variant, modification, homologue, derivative or subsequence thereof has replaced the mucin-like domain (MLD) of the Ebola virus glycoprotein. It is within the scope of the present disclosure to make use of any filovirus glycoprotein or any mutant thereof, including any glycoprotein or mutant thereof from the filovirus genus, or any glycoprotein or mutant thereof from the Marburg or Ebola sero/genotypes. Such Coronavirus and Ebola virus antigens and epitopes set forth herein or known to one skilled in the art include a Coronavirus or Ebola virus antigen that increases, stimulates, enhances, promotes, augments or induces a proinflammatory or adaptive immune response, numbers or activation of an immune cell (e.g., T cell, natural killer T (NKT) cell, dendritic cell (DC), B cell, macrophage, neutrophil, eosinophil, mast cell, CD4⁺ or a CD8⁺ cell, B220⁺ cell, CD14⁺, CD11b⁺ or CD11c⁺ cells), an anti-Coronavirus or anti-Ebola virus B cell, CD4⁺ T cell or CD8⁺ T cell response, production of a Th1 cytokine, a T cell mediated immune response, a B cell mediated immune response etc.

Anti-viral antigens and epitopes set forth herein or known to one skilled in the art include a viral antigen that increases, stimulates, enhances, promoters, augments or induces a proinflammatory or adaptive immune response, numbers or activation of an immune cell (e.g., T cell, natural killer (NKT) cell, dendritic cell (DC), B cell, macrophage, neutrophil, eosinophil, mast cell, CD4⁺ or CD8⁺ cell B220⁺ cell, CD14⁺ cell, CD11b⁺ or CD11⁺ cells), an viral B cell, CD4⁺ T cell or CD8⁺ T cell response, production of Th1 cytokine, a T cell mediated immune response, a B cell mediated immune response etc.

Combination methods and use embodiments include, for example, second actives such as anti-pathogen drugs, such as protease inhibitors, reverse transcriptase inhibitors, virus fusion inhibitors and virus entry inhibitors, antibodies to pathogen proteins, live or attenuated pathogen, or a nucleic acid encoding all or a portion (e.g., an epitope) of any protein or proteinaceous pathogen antigen, immune stimulating agents, etc., and include contact with, administration in vitro or in vivo, with another compound, agent, treatment or therapeutic regimen appropriate for pathogen infection, vaccination or immunization

In certain instances, as will be apparent to a person of skill in the art, references to a particular viral protein (e.g., a Coronavirus or Ebola virus protein), peptide, or a variant, modification, homologue, derivative or subsequence thereof as used herein also encompasses a nucleic acid molecule encoding a viral protein, peptide, or the variant, modification, homologue, derivative or subsequence thereof (e.g., a Coronavirus or Ebola virus protein, peptide, or the variant, modification, homologue, derivative or subsequence thereof). For example, descriptions methods and composition of the present invention comprising administration of a viral protein, peptide, or a variant, modification, homologue, derivative or subsequence thereof (e.g., a Coronavirus or Ebola virus protein, peptide, or a variant, modification, homologue, derivative or subsequence thereof) encompasses administration of a nucleic acid molecule encoding a viral protein, peptide or a variant, modification, homologue, derivative or subsequence thereof (e.g., the Coronavirus or Ebola virus protein, peptide, or the variant, modification, homologue, derivative or subsequence thereof).

Methods of the present disclosure also include, among other things, methods that result in a reduced need or use of another compound, agent, drug, therapeutic regimen, treatment protocol, process, or remedy. For example, for a particular viral infection or pathology, vaccination or immunization (e.g., a Coronavirus or Ebola virus infection or pathology, vaccination or immunization), a method of the invention has a therapeutic benefit if in a given subject a less frequent or reduced dose or elimination of an anti-viral treatment (e.g., anti-Coronavirus or anti-Ebola virus treatment) results. Thus, in accordance with the invention, methods of reducing need or use of a treatment or therapy for a particular viral infection or pathology, or vaccination or immunization (e.g., a Coronavirus or Ebola virus infection or pathology, or vaccination or immunization), are provided.

In methods disclosed herein in which there is a desired outcome, such as a therapeutic or prophylactic method that provides a benefit from treatment, vaccination or immunization against Coronavirus and/or Ebola virus protein, peptide, or a variant, modification, homologue, derivative or subsequence thereof can be administered in a sufficient or effective amount.

Without limiting any of the foregoing, the following exemplification of carriers, modes of administration, dosage forms, etc., are listed as known possibilities from which the carriers, modes of administration, dosage forms, etc., may be selected for use with the present invention. Those of ordinary skill in the art will understand, however, that any given formulation and mode of administration selected should first be tested to determine that it achieves the desired results.

Methods of administration include, but are not limited to, parenteral, e.g., intravenous, intraperitoneal, intramuscular, subcutaneous, mucosal (e.g., oral, intranasal, buccal, vaginal, rectal, intraocular), intrathecal, topical and intradermal routes. Administration can be systemic or local.

The compositions of the present disclosure may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen free water, before use.

For instance, the composition of the present disclosure may be administered in the form of an injectable preparation, such as sterile injectable aqueous or oleaginous suspensions. These suspensions may be formulated according to techniques known in the art using suitable dispersing or wetting agents and suspending agents. The sterile injectable preparations may also be sterile injectable solutions or suspensions in non-toxic parenterally-acceptable diluents or solvents. They may be given parenterally, for example intravenously, intramuscularly or sub-cutaneously by injection, by infusion or per os. Suitable dosages will vary, depending upon factors such as the amount of each of the components in the composition, the desired effect (short or long term), the route of administration, the age and the weight of the subject to be treated. Any other methods well known in the art may be used for administering the composition of the present disclosure.

The composition of the present disclosure may be formulated as a dry powder (i.e., in lyophilized form). Freeze-drying (also referred to as lyophilisation) is often used for preservation and storage of biologically active material because of the low temperature exposure during drying. Typically, the liquid antigen is freeze dried in the presence of agents to protect the antigen during the lyophilization process and to yield a cake with desirable powder characteristics. Sugars such as sucrose, mannitol, trehalose, or lactose (present at an initial concentration of 10-200 mg/mL) are commonly used for cryoprotection of protein antigens and to yield lyophilized cake with desirable powder characteristics. Lyophilizing the composition theoretically results in a more stable composition.

In certain embodiments, the composition of the present disclosure may be formulated as a liquid (e.g., aqueous formulation), e.g., as syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The pharmaceutical compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art.

Where the composition of the present disclosure is intended for delivery to the respiratory (e.g., nasal) mucosa, typically it is formulated as an aqueous solution for administration as an aerosol or nasal drops, or alternatively, as a dry powder, e.g., for rapid deposition within the nasal passage. Compositions for administration as nasal drops may contain one or more excipients of the type usually included in such compositions, for example preservatives, viscosity adjusting agents, tonicity adjusting agents, buffering agents, and the like. Viscosity agents can be microcrystalline cellulose, chitosan, starches, polysaccharides, and the like. Compositions for administration as dry powder may also contain one or more excipients usually included in such compositions, for example, mucoadhesive agents, bulking agents, and agents to deliver appropriate powder flow and size characteristics. Bulking and powder flow and size agents may include mannitol, sucrose, trehalose, and xylitol.

### EXAMPLES

### Example 1. Summary of Aspects of Invention

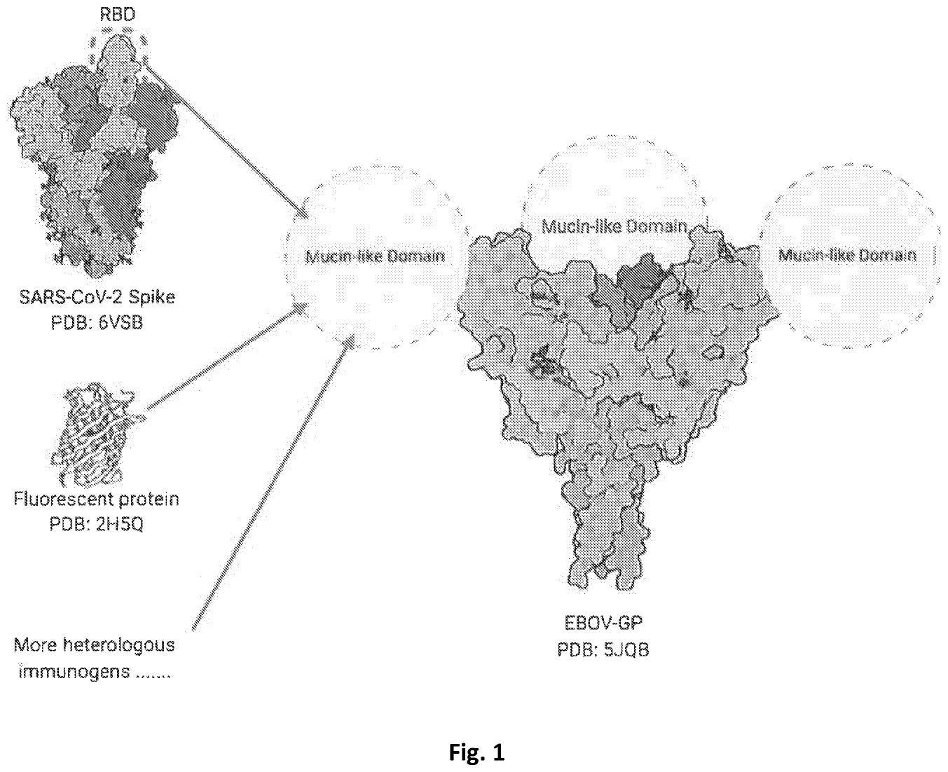

The glycoprotein (GP) of Ebola virus (EBOV), which sufficiently elicits protective immune response in animals and human beings, is the key immunogen of the licensed vaccine against EBOV (Ervebo). The mucin-like domain (MLD) of EBOV GP is a highly variable domain and structurally independent from the main GP trimer. It is one of the immunodominant regions that are exposed on the surface of the EBOV GP trimer, however, the antibodies elicited by MLD are usually not cross-reactive are less likely to be protective. In the present disclosure, the inventors engineered the EBOV GP as a novel immunogen display platform by replacing the MLD with heterologous immunogens, which could be developed as multi-valent vaccines against emerging and re-emerging pathogens. The design of the recombinant EBOV-GPs may be used as protein-based subunit vaccines or carried on the vaccine vectors. Here, the inventors demonstrate and explain the design by displaying the receptor binding domain (RBD) of SARS-COV-2 spike protein and mCherry fluorescent protein on the glycoprotein of Zaire Ebolavirus (ZEBOV) as two examples (see, for e.g.: FIG. 1).

### Example 2. Bivalent Vaccine Against Both Zaire Ebolavirus (ZEBOV) and Severe Acute Respiratory Syndrome Coronavirus 2 (SARS-COV-2

The receptor-binding domain (RBD) of SARS-COV-2 spike protein initiates viral entry by interacting with the human cellular receptor angiotensin-converting enzyme-2 (hACE2). Immunization with recombinant RBD protein elicited neutralizing antibodies without detectable antibody-dependent enhancement (ADE) in animal models. Therefore, the RBD is an ideal immunogen for SARS-COV-2 vaccine. However, as a protein-based subunit vaccine candidate, its immunogenicity may not be strong enough to achieve full protection. Besides, the epidemic regions of current SARS-COV-2 in Africa overlap with the regions of the historical EBOV outbreaks. The novel immunogen display approach details herein may be developed as an efficient bivalent vaccine against both EBOV and SARS-COV-2.

### Example 2.1. Structural and Functional Verification of the Recombinant ZEBOV-GP-deltaMLD-SARS-COV-2-RBD

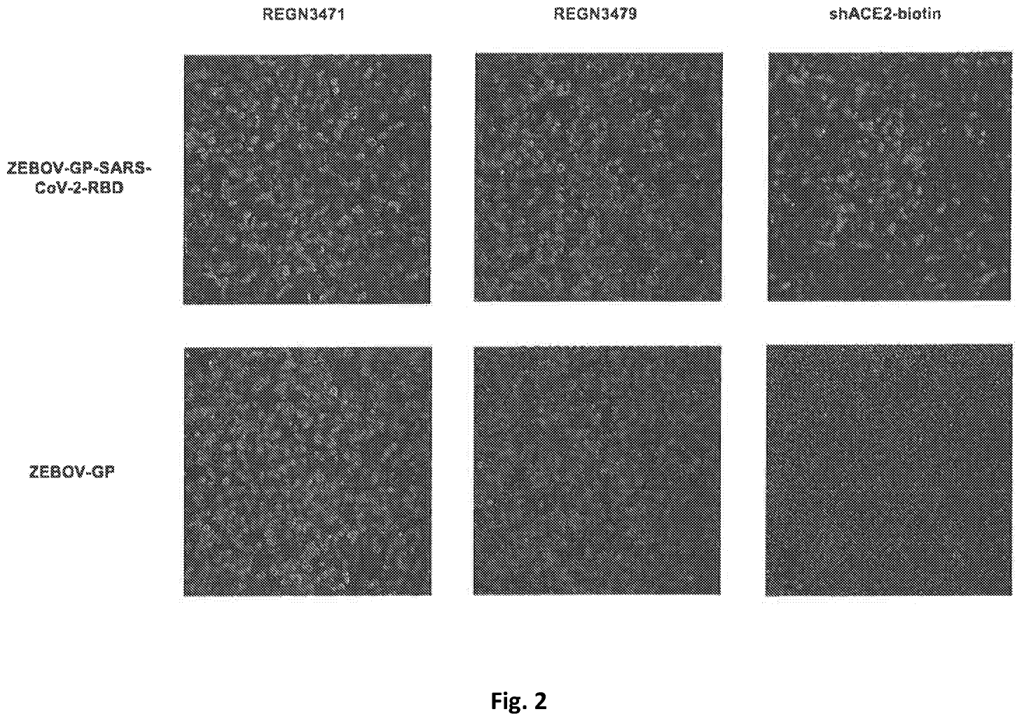

Based on the structural information, the inventors replaced the ZEBOV-MLD (314-462 aa) by SARS-COV-RBD (328-535 aa), termed as ZEBOV-GP-deltaMLD-SARS-COV-2-RBD. The recombinant glycoprotein was expressed on the HEK293T cell surface and immunofluorescent assays were performed to check its conformation. Two anti-EBOV neutralizing antibodies (REGM3471, REGM3479) against different GP epitopes and biotin-labeled soluble human ACE2 (shACE2-biotin) were used for the test. The ZEBOV-GP-deltaMLD-SARS-COV-2-RBD on the cell surface could be recognized by both the antibodies and purified human ACE2 (see, for e.g.: FIG. 2). FIG. 2 demonstrates that the ZEBOV-GP-deltaMLD-SARS-CoV-2-RBD recognized by anti-EBOV-GP neutralizing antibodies (REGN3471 and REGN3479) and purified soluble human ACE2 (shACE2).

### Example 2.2. Building the Recombinant Virus-Based Vaccine Vectors Vesicular Embedding ZEBOV-GP-deltaMLD-SARS-COV-2-RBD

The currently available EBOV vaccine (Ervebo) is a replication-competent recombinant vesicular stomatitis virus (rVSV) with ZEBOV-GP displayed on the surface of the virion. To test whether the ZEBOV-GP-deltaMLD-SARS-COV-2-RBD is able to serve as the glycoprotein of rVSV mediating viral entry, the inventors made the ZEBOV-GP-deltaMLD-SARS-COV-2-RBD pseudotyped VSV (single-round infection with GFP as the reporter gene). The transduction activity of rVSV-ZEBOV-GP-deltaMLD-SARS-COV-2-RBD-PsV in Vero cell line was 1.25E+05 (+/−1.69E+04) FFU/mL, while the rVSV-ZEBOV-PSV was 0.80E+05 (+/−1.70E+05) FFU/mL. The considerable transduction activity of pseudotyped rVSV indicated the potential of replication-competent rVSV-ZEBOV-GP-deltaMLD-SARS-COV-2-RBD to be developed as a bivalent vaccine.

In general, the data demonstrated that the ZEBOV-GP-deltaMLD-SARS-COV-2-RBD could be correctly displayed on the EBOV-GP without impairing the GP function. It is specifically intended that the replication-competent rVSV-ZEBOV-GP-deltaMLD-SARS-COV-2-RBD can be rescued and the immunogenicity and safety can be further investigated in animal models.

### Example 3. Fluorescent Protein Functionally Displayed on ZEBOV-GP

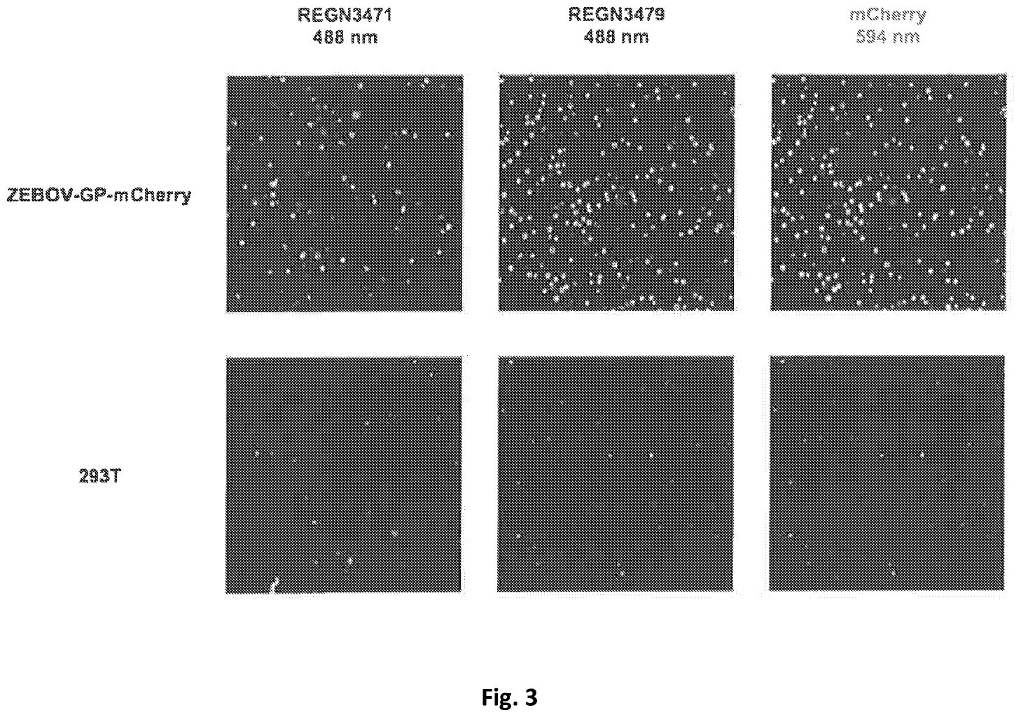

Similar to Example 2, the inventors replaced ZEBOV-MLD with mCherry fluorescent protein, and verified that the recombinant GP trimers were able to be recognized by the anti-EBOV antibodies, while the displayed mCherry could be excited correctly (see, for e.g.: FIG. 3). The results in this Example showed that the EBOV-GP was able to correctly display diverse heterologous proteins (immunogens) without impairing the conformation of the main GP trimer. FIG. 3 demonstrates ZEBOV-GP-deltaMLD-mCherry recognized by anti-EBOV-GP neutralizing antibodies (REGN3471 and REGN3479).

### Example 4. Vaccine Vectors Using Zaire Ebolavirus Glycoprotein

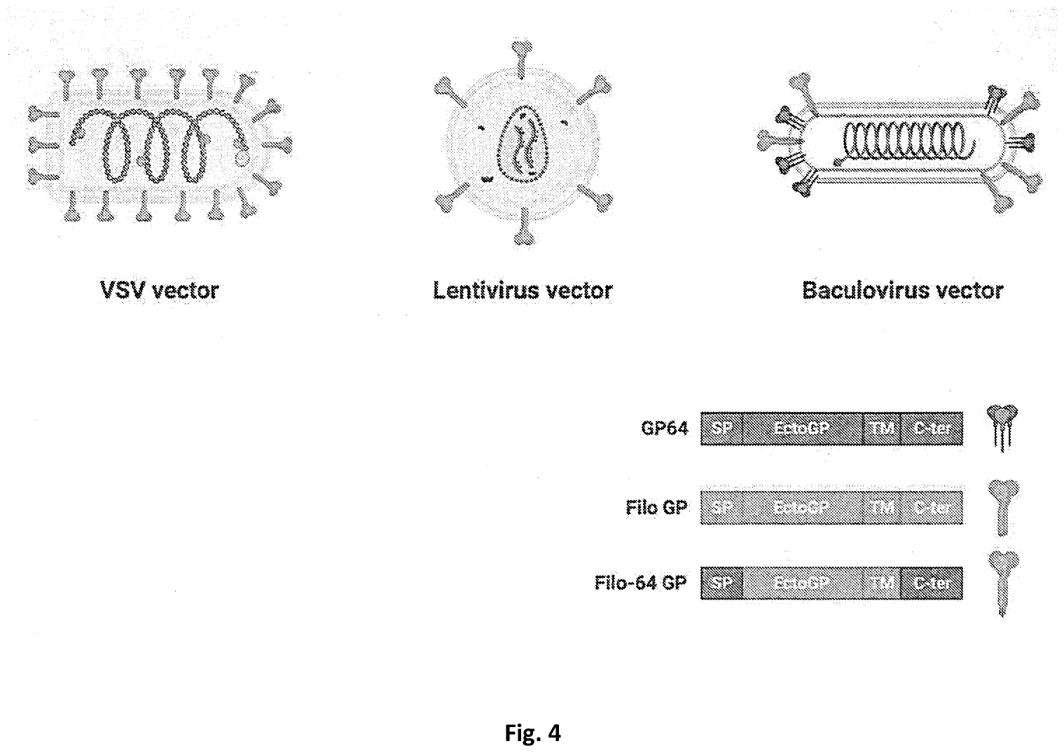

To verify the antigen design in other vaccine vectors, recombinant replication-competent VSV, lentivirus and baculovirus displaying ZEBOV-GP-deltaMLD-SARS-COV-2-RBD were developed as shown generally in FIG. 4. As shown in FIG. 4, three vaccine vectors displaying ZEBOV-GP-deltaMLD-SARS-COV-2-RBD were developed. For baculovirus surface display, the signal peptide (SP) and cytoplasmic domain (C-ter) of ZEBOV GP were replaced by the counterparts of baculovirus GP64.

With respect to the lentiviral vector, the transduction activity of lentiviral pseudovirus (tested in HEK293T-ACE2 cell line was as follows:
Lenti-ZEBOV-PsV: 0.32E+04 (+/−0.43E+03) FFU/mL
Lenti-ZEBOV-GP-deltaMLD-SARS-COV-2-RBD-PsV: 6.97E+04 (+/−4.82E+03) FFU/mL

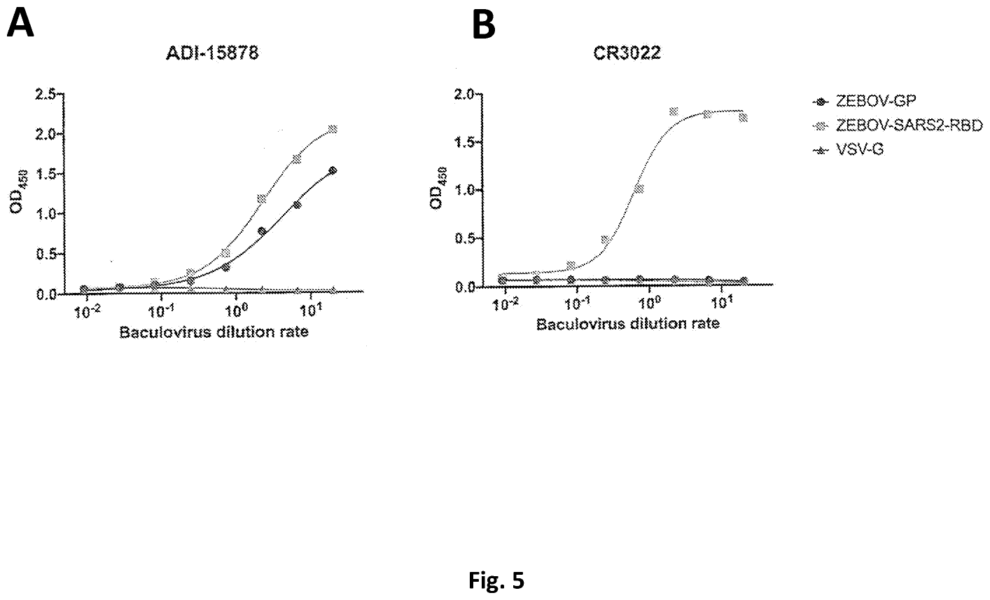

With respect to the baculovirus vector, both ZEBOV-GP and ZEBOV-GP-deltaMLD-SARS-COV-2-RBD can be displayed on the surface of baculovirus with correct conformation (see, for e.g.: FIG. 5). More specifically, and as shown in FIG. 5, ELISA binding of baculovirus and monoclonal antibodies is detailed therein. Baculoviruses displaying three different GPs respectively; anti-Ebola neutralizing antibody (ADI-15878; see FIG. 5a) and anti-SARS-COV-2-RBD conformational antibody (CR3022; see FIG. 5b) were used to verify the conformation of ZEBOV-GP and RBD.

### Example 4.1. Immunogenicity of ZEBOV-GP-deltaMLD-SARS-COV-2-RBD Displaying on Baculovirus

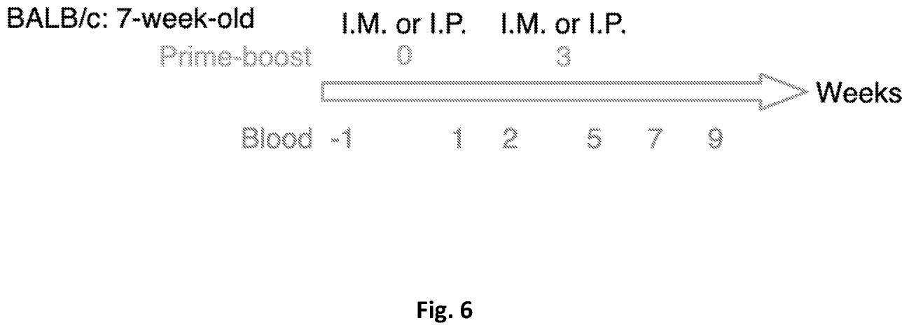

BALB/c mice were immunized twice (at week 0 and week 3) with recombinant baculovirus intramuscularly or intraperitoneally (10\hat{ }9 baculovirus per mouse per dose). No adjuvant was applied in the experiment. Serum of each mouse was collected at week −1, 1, 2, 4, 5, 7, 9 (FIG. 6).

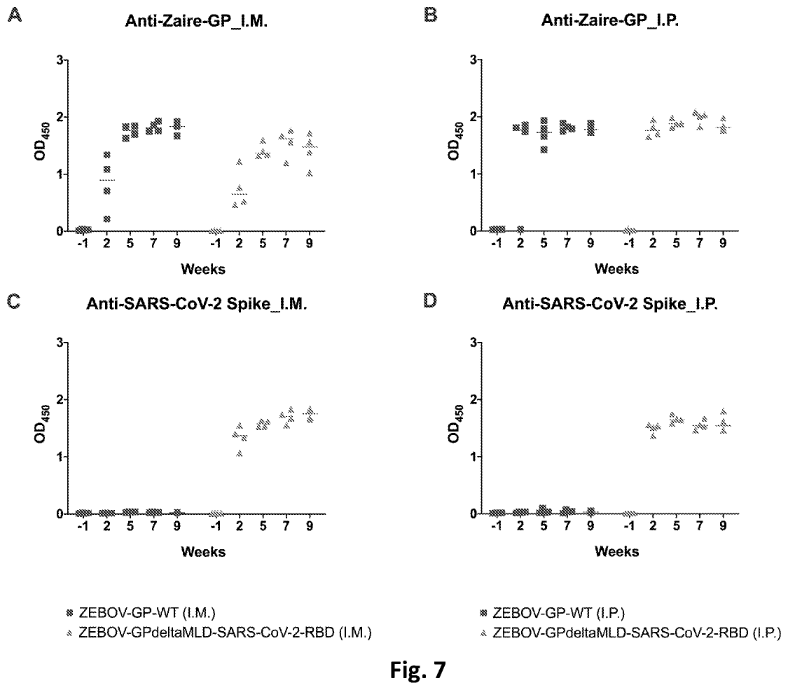

Both ZEBOV-GP and ZEBOV-GP-deltaMLD-SARS-COV-2-RBD recombinant baculoviruses potently elicited specific IgG against ZEBOV-GP after two doses immunization (Week 5), meanwhile, anti-SARS-COV-2-Spike IgGs were only detected from the mice immunized with the recombinant baculovirus displaying ZEBOV-GP-deltaMLD-SARS-COV-2-RBD. The level of anti-SARS-COV-2-Spike IgG reached a high level after prime (Week 2), and lasted through the experiment (Week 2-9). The two vaccination routes didn't show significant difference in IgG elicitation (FIG. 7).

More specifically and as shown in FIG. 7, total IgG against ZEBOV-GP or SARS-COV-2-Spike in mice sera immunized with recombinant baculovirus were measured by ELISA (n=4 or 5). 100 ng/well purified ZEBOV-GP or SARS-COV-2-Spike were coated on the plate. The serum of each mouse collected at week −1 (before prime), 2 (after prime), 5 (after boost), 7, and 9 were added to the antigen with 100 times dilution. HRP-conjugated anti-mouse-IgG antibody was used as the secondary antibody, and the readouts were recorded at OD 450. I.M: intramuscular injection, I.P: intraperitoneal injection.

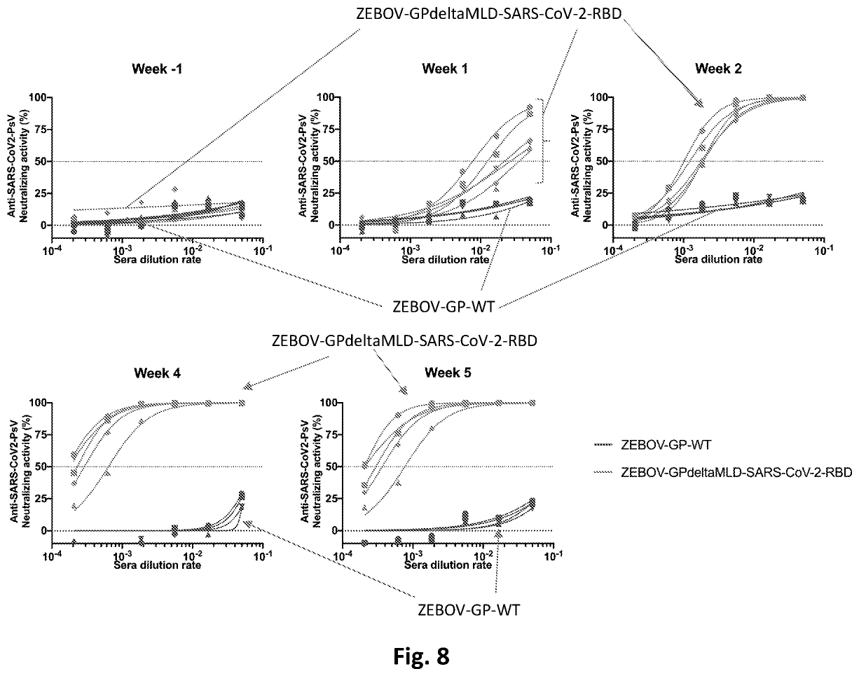

Sera from intraperitoneally immunized mice were further tested for neutralizing activity against rVSV-based SARS-COV-2 pseudovirus. The sera anti-SARS-COV-2 neutralizing activity of mice immunized with recombinant baculovirus displaying ZEBOV-GP-deltaMLD-SARS-COV-2-RBD increased from one week after prime vaccination (Week 1), and reached a high level after boost (Week 4 and 5). As the wild type ZEBOV-GP control group, no anti-SARS-COV-2 neutralizing activity was found in mice vaccinated with ZEBOV-GP baculovirus (FIG. 8).

More specifically and as shown in FIG. 8, neutralizing activity was tested against rVSV-based SARS-COV-2 pseudovirus in sera of recombinant baculovirus immunized mice. The serum of each mouse (n=4 or 5) collected at week −1 (before prime), 1, 2 (after prime), 4, 5 (after boost) were serially diluted and mixed with SARS-COV-2 pseudovirus (encoding GFP reporter gene) before being added to the Vero cells. Eighteen hours after transduction, GFP-positive cells were counted for neutralization activity calculation. Horizontal dotted line indicates 50% neutralization.

These mouse immunization results demonstrated the ZEBOV-GP could correctly display SARS-COV-2-RBD on it, and the chimeric immunogen could elicit specific IgGs against both parts of ZEBOV-GP and RBD in mice. Importantly, anti-SARS-COV-2 neutralizing antibodies were potently elicited, which verified the concept of this novel bivalent vaccine design. This is the first recorded instance of the Filovirus-GP being displayed on the baculovirus surface.

This Example demonstrates how potent the neutralizing response was in circumstances where no adjuvant was included, and further demonstrates that the combination of baculovirus vector and/or use of the SARS-COV-2-RBD has improved binding to APCs thus resulting in a better immune response overall (including a better neutralizing response) even with no adjuvant. The rationale for this Example is underpinned on the universal expression of ACE2 receptor in certain cells, including APCs (e.g., lung macrophages and dendritic cells etc.) and other immune cells, and ACE2 receptor is the target of RBD. As demonstrated herein, including the RBD and presenting the composition to APCs results in a more potent immune response.

### Example 5. Vaccine Vectors from Other Filoviruses

GPs from other filoviruses displaying RBDs from different coronaviruses were also developed and tested. By using a similar strategy to that described herein, the following chimeric GPs were constructed:
Marburg virus (Angola strain): MARV-GP-deltaMLD-SARS-COV-2-RBD
Sudan ebolavirus: SUDV-GP-deltaMLD-SARS-COV-1-RBD
Bundibugyo ebolavirus: BDBV-GP-deltaMLD-MERS-COV-RBD

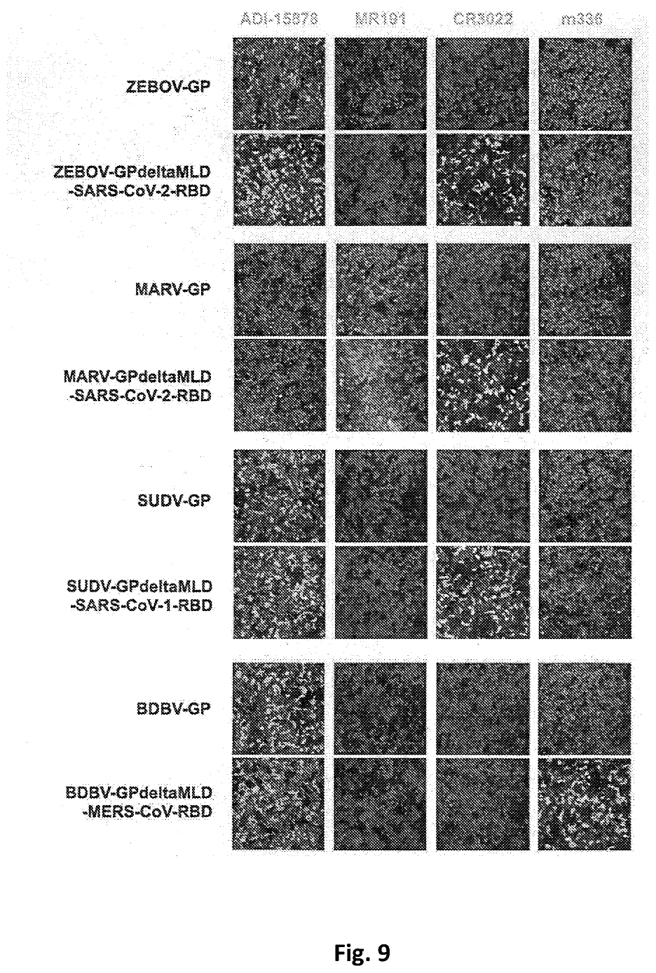

The aforementioned chimeric GPs were tested and results of an immunofluorescence assay are detailed in FIG. 9. GPs were expressed on the surface of HEK293T cells. Pan-Ebola neutralizing antibody (ADI-15878), anti-MARV neutralizing antibody (MR191) and anti-SARS-CoV-2-RBD and SARS-COV-1-RBD conformational antibody (CR3022) and anti-MERS-COV-RBD neutralizing antibody (m336) were used to verify the conformation of GPs and RBD.

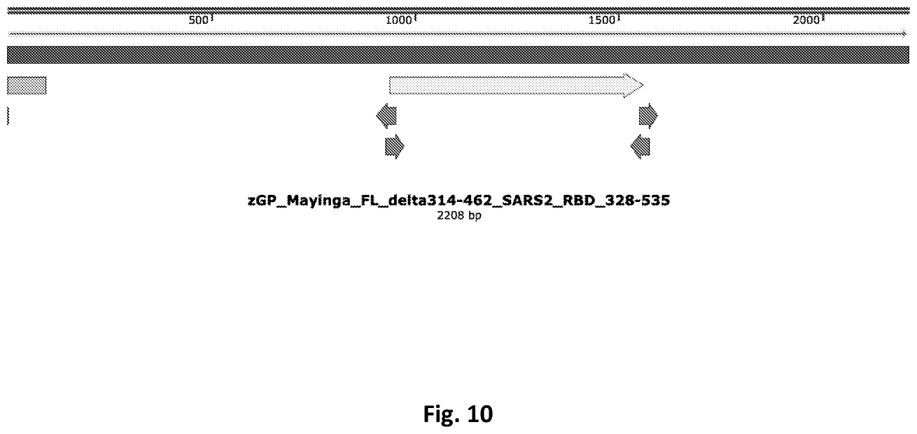

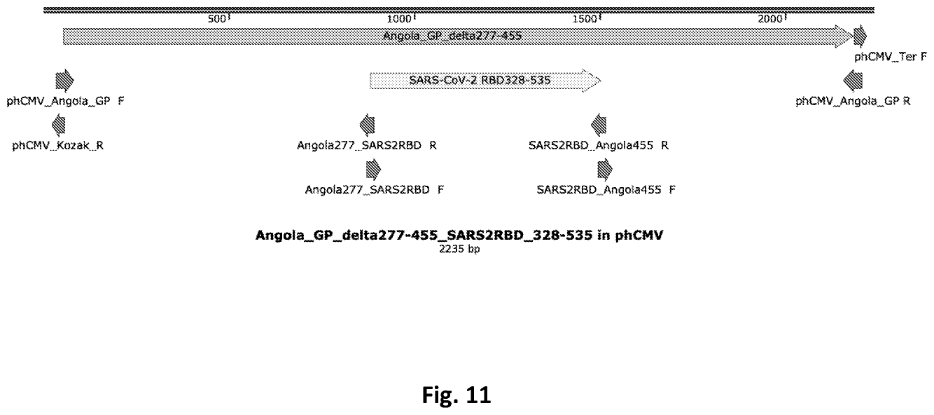

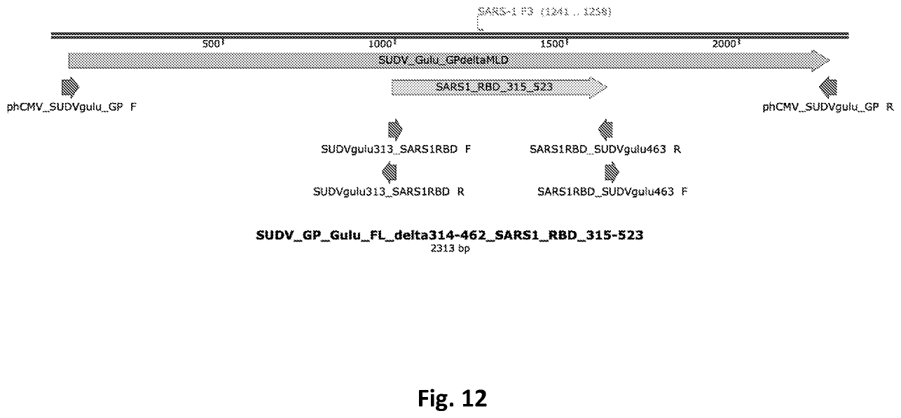

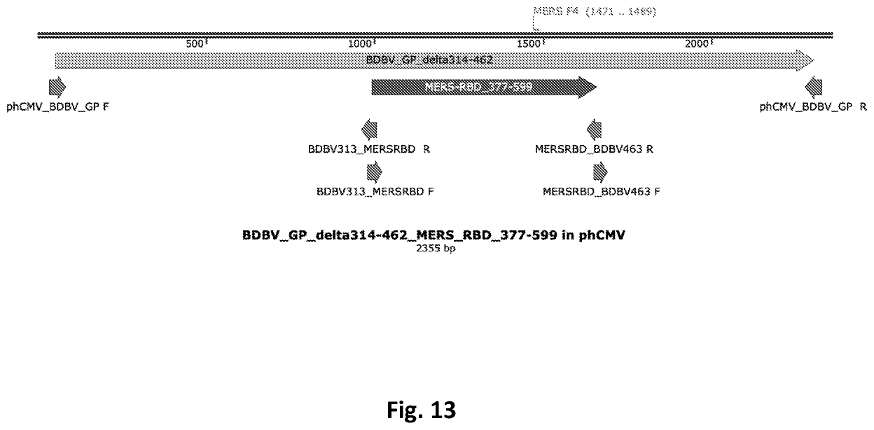

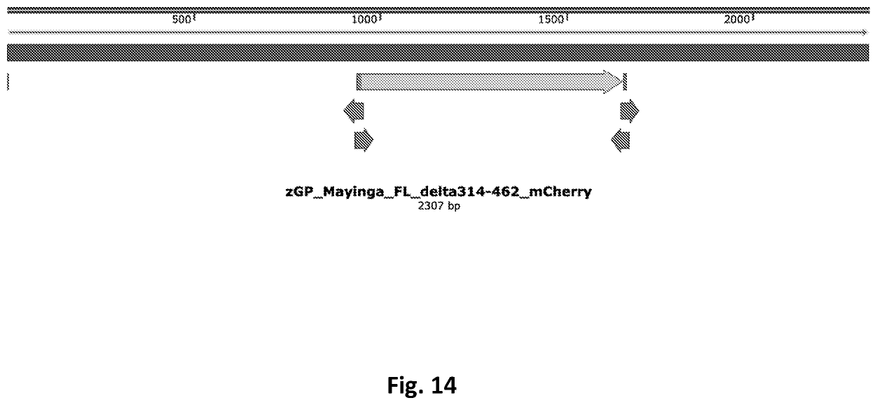

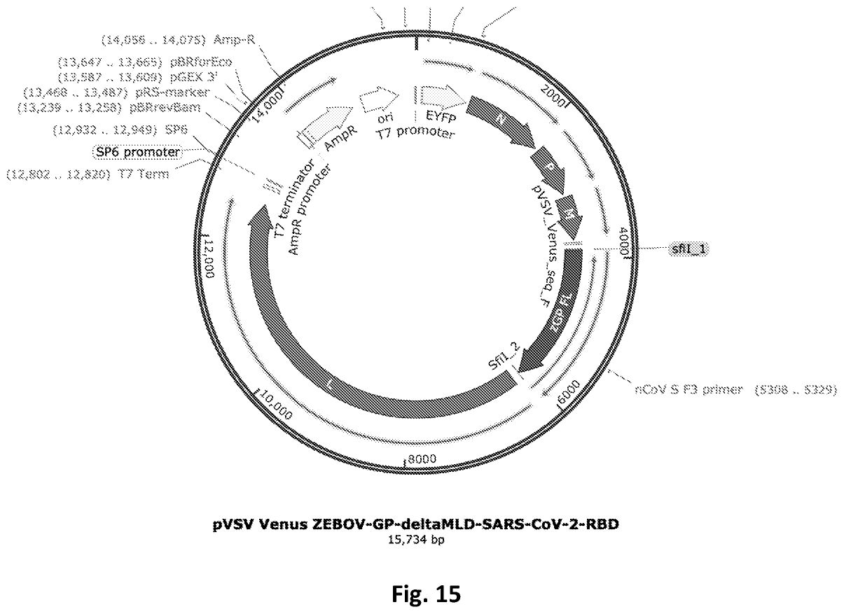

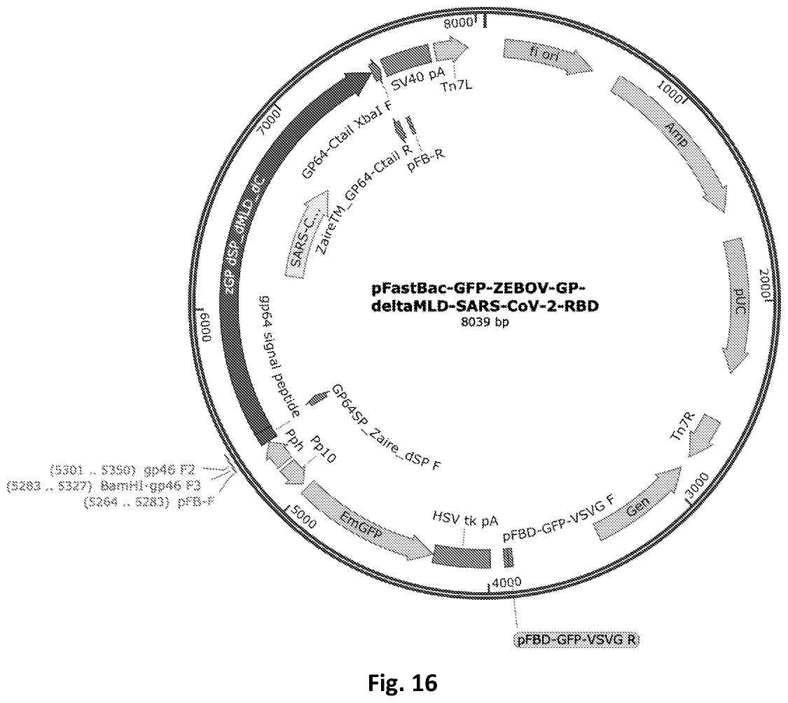

The DNA sequences of the chimeric GPs are depicted in FIGS. 10-16.

Although various embodiments of the disclosure have been described and illustrated, it will be apparent to those skilled in the art in light of the present description that numerous modifications and variations can be made. The scope of the invention is defined more particularly in the appended claims.

### Sequences

The following sequences are referred to herein:

| SEQ | | |
|---|---|---|
| ID | | |
| NO: | Description | Sequence |
| 1 | ZEBOV-GP- | ATGGGCGTGACTGGAATCCTGCAGCTGCCAAGAGATCGGTTCAAGC |
| | deltaMLD- | GGACTTCATTTTTCCTGTGGGTCATCATCCTGTTTCAGAGAACCTTTT |
| | SARS-CoV-2- | CTATTCCTCTTGGCGTTATCCATAATAGTACTCTCCAAGTCAGCGAC |
| | RBD | GTCGATAAACTTGTTTGCCGCGATAAACTTTCCAGTACTAACCAGCT |
| | | GCGCTCCGTGGGTCTTAACCTCGAAGGAAACGGAGTTGCCACCGAT |
| | | GTTCCCTCCGCCACTAAGCGCTGGGGCTTCCGCTCCGGAGTCCCACC |
| | | CAAGGTAGTGAACTATGAGGCGGGCGAATGGGCCGAGAACTGTTAT |
| | | AATCTTGAGATAAAAAAACCCGACGGATCCGAGTGCCTTCCAGCTG |
| | | CCCCGGATGGTATTAGGGGCTTCCCACGCTGTCGCTACGTTCATAAA |
| | | GTATCCGGAACCGGACCTTGTGCTGGAGACTTCGCATTCCACAAAGA |
| | | GGGCGCGTTTTTTCTGTATGATCGCCTTGCATCCACCGTAATCTACCG |
| | | CGGCACGACCTTTGCTGAAGGTGTGGTCGCTTTCCTTATTCTCCCAC |
| | | AAGCTAAGAAGGACTTTTTCTCCTCCCACCCACTCCGCGAGCCAGTA |
| | | AACGCAACTGAAGATCCCTCCAGCGGTTACTACTCCACCACCATTCG |
| | | CTACCAGGCAACCGGATTTGGAACAAATGAAACAGAGTACCTGTTC |
| | | GAAGTTGATAATCTGACATACGTCCAGCTTGAATCCCGCTTCACTCC |
| | | CCAGTTTTTGCTGCAACTCAACGAAACAATATACACATCCGGCAAGC |
| | | GCTCCAACACCACCGGCAAATTGATATGGAAAGTAAACCCTGAAAT |
| | | TGACACAACTATCGGAGAATGGGCATTCTGGGAAACAAAAAAAAAT |
| | | CTCACTCGCAAAATTAGGTCCGAGGAACTCAGCTTCACAGTAGTTTC |
| | | CAATCGCTTTCCTAATATCACAAACCTGTGCCCATTTGGCGAGGTGT |
| | | TCAACGCAACCCGCTTCGCCAGCGTGTACGCCTGGAATAGGAAGCG |
| | | GATCAGCAACTGCGTGGCCGACTATAGCGTGCTGTACAACTCCGCCT |
| | | CTTTCAGCACCTTTAAGTGCTATGGCGTGTCCCCCACAAAGCTGAAT |
| | | GACCTGTGCTTTACCAACGTCTACGCCGATTCTTTCGTGATCAGGGG |
| | | CGACGAGGTGCGCCAGATCGCCCCCGGCCAGACAGGCAAGATCGCA |
| | | GACTACAATTATAAGCTGCCAGACGATTTCACCGGCTGCGTGATCGC |
| | | CTGGAACAGCAACAATCTGGATTCCAAAGTGGGCGGCAACTACAAT |
| | | TATCTGTACCGGCTGTTTAGAAAGAGCAATCTGAAGCCCTTCGAGAG |
| | | GGACATCTCTACAGAAATCTACCAGGCCGGCAGCACCCCTTGCAAT |
| | | GGCGTGGAGGGCTTTAACTGTTATTTCCCACTCCAGTCCTACGGCTT |
| | | CCAGCCCACAAACGGCGTGGGCTATCAGCCTTACCGCGTGGTGGTG |
| | | CTGAGCTTTGAGCTGCTGCACGCCCCAGCAACAGTGTGCGGCCCCAA |
| | | GAAGTCCACCAATCTGGTGAAGAACACCCACCATCAGGACACAGGA |
| | | GAGGAATCCGCATCCTCCGGTAAACTTGGTTTGATAACAAACACCAT |
| | | CGCAGGCGTAGCTGGACTGATAACAGGCGGACGCCGCACCAGGAGG |
| | | GAAGCTATTGTCAACGCTCAACCAAAATGCAACCCCAACCTTCACTA |
| | | TTGGACCACTCAGGATGAAGGAGCCGCTATAGGACTCGCATGGATA |
| | | CCTTACTTCGGTCCCGCAGCTGAAGGCATATACACCGAAGGACTGAT |
| | | GCATAACCAAGACGGTCTGATTTGTGGTCTTAGGCAGCTGGCAAAC |
| | | GAAACCACTCAGGCCCTGCAGCTGTTCTTGCGCGCTACGACAGAACT |
| | | CCGCACCTTTAGTATCTTGAACCGCAAAGCTATTGATTTCCTTCTTCA |
| | | ACGCTGGGGCGGCACCTGCCACATCCTGGGACCCGATTGCTGTATTG |
| | | AACCCCACGACTGGACCAAAAACATCACAGACAAGATAGATCAGAT |
| | | AATCCACGATTTTGTAGACAAGACCCTCCCAGACCAAGGTGACAAT |
| | | GACAATTGGTGGACAGGTTGGCGCCAATGGATCCCTGCAGGCATTG |
| | | GTGTAACAGGAGTAATCATAGCCGTTATCGCCCTGTTTTGTATTTGT |
| | | AAATTCGTGTTTTGA |
| 2 | MARV-GP- | ATGAAAACCACCTGCCTGCTGATCAGCCTGATCCTGATCCAGGGCGT |
| | deltaMLD- | GAAAACCCTGCCCATCCTGGAAATCGCCAGCAACATCCAGCCCCAG |
| | SARS-CoV-2- | AACGTGGACAGCGTGTGCAGCGGCACCCTGCAGAAAACCGAGGACG |
| | RBD | TGCACCTGATGGGCTTCACACTGAGCGGCCAGAAGGTGGCCGACAG |
| | | CCCCCTGGAAGCCAGCAAGAGATGGGCCTTCAGAGCCGGCGTGCCC |
| | | CCCAAGAATGTGGAGTACACCGAAGGCGAGGAAGCCAAGACCTGCT |
| | | ACAACATCAGCGTGACCGACCCCAGCGGCAAGAGCCTGCTGCTGGA |
| | | CCCCCCCACCAACATCAGAGACTACCCCAAGTGCAAGACCATCCAC |
| | | CACATCCAGGGCCAGAACCCCCACGCCCAGGGCATTGCTCTGCACCT |
| | | GTGGGGCGCCTTTTTCCTGTACGACCGGATCGCCAGCACCACCATGT |
| | | ACCGGGGCAAGGTGTTCACCGAGGGCAATATCGCCGCCATGATCGT |
| | | GAACAAGACCGTGCACAAGATGATCTTCAGCAGACAGGGCCAGGGC |
| | | TACCGGCACATGAACCTGACCAGCACCAACAAGTACTGGACCAGCA |
| | | GCAACGGCACCCAGACCAACGACACCGGCTGCTTCGGCACACTGCA |
| | | GGAATACAACAGCACCAAGAACCAGACCTGCGCCCCCAGCAAGAAG |
| | | CCCCTGCCCCTGCCTACCGCCCACCCCGAAGTGAAGCTGACCTCCAC |
| | | CTCCACCGACGCCACCAAGCTGAACACCACCGACCCCAACAGCGAC |
| | | GACGAGGACCTGACCACCTCCGGCAGCGGCAGCGGCGAGCAGCGCT |
| | | TTCCTAATATCACAAACCTGTGCCCATTTGGCGAGGTGTTCAACGCA |
| | | ACCCGCTTCGCCAGCGTGTACGCCTGGAATAGGAAGCGGATCAGCA |
| | | ACTGCGTGGCCGACTATAGCGTGCTGTACAACTCCGCCTCTTTCAGC |
| | | ACCTTTAAGTGCTATGGCGTGTCCCCCACAAAGCTGAATGACCTGTG |
| | | CTTTACCAACGTCTACGCCGATTCTTTCGTGATCAGGGGCGACGAGG |
| | | TGCGCCAGATCGCCCCCGGCCAGACAGGCAAGATCGCAGACTACAA |
| | | TTATAAGCTGCCAGACGATTTCACCGGCTGCGTGATCGCCTGGAACA |
| | | GCAACAATCTGGATTCCAAAGTGGGCGGCAACTACAATTATCTGTAC |
| | | CGGCTGTTTAGAAAGAGCAATCTGAAGCCCTTCGAGAGGGACATCT |
| | | CTACAGAAATCTACCAGGCCGGCAGCACCCCTTGCAATGGCGTGGA |
| | | GGGCTTTAACTGTTATTTCCCACTCCAGTCCTACGGCTTCCAGCCCAC |
| | | AAACGGCGTGGGCTATCAGCCTTACCGCGTGGTGGTGCTGAGCTTTG |
| | | AGCTGCTGCACGCCCCAGCAACAGTGTGCGGCCCCAAGAAGTCCAC |
| | | CAATCTGGTGAAGATCGACTTCGACCCCGTGCCTAACACCAAGACC |
| | | ATCTTCGACGAATCTTCATCTTCCGGAGCTTCAGCTGAAGAGGACCA |
| | | GCACGCCAGCCCCAACATCTCCCTGACCCTGAGCTACTTCCCCAAAG |
| | | TGAACGAGAACACCGCCCACAGCGGCGAGAACGAGAACGACTGCG |
| | | ACGCCGAGCTGCGGATTTGGAGCGTGCAGGAAGATGACCTGGCCGC |
| | | CGGACTGAGCTGGATCCCCTTCTTCGGCCCTGGCATCGAGGGCCTGT |
| | | ACACAGCCGGCCTGATTAAGAACCAGAACAACCTGGTGTGCCGGCT |
| | | GCGGCGGCTGGCCAACCAGACCGCCAAGTCCCTGGAACTGCTGCTG |
| | | AGAGTGACCACCGAGGAACGGACCTTCTCCCTGATCAACCGGCACG |
| | | CCATCGATTTTCTGCTGGCCAGATGGGGCGGCACCTGCAAGGTGCTG |
| | | GGCCCCGACTGCTGCATCGGCATCGAGGATCTGAGCCGGAACATCA |
| | | GCGAGCAGATCGACCAGATCAAGAAGGACGAGCAGAAAGAGGGCA |
| | | CCGGCTGGGGCCTGGGCGGCAAGTGGTGGACCAGCGACTGGGGCGT |
| | | GCTGACCAACCTGGGCATCCTGCTGCTGCTGTCTATCGCCGTGCTGA |
| | | TCGCCCTGAGCTGCATCTGCCGGATCTTCACCAAGTACATCGGCTGA |
| 3 | SUDV-GP- | ATGGGGGGTCTTAGCCTACTCCAATTGCCCAGGGACAAATTTCGGAA |
| | deltaMLD- | AAGCTCTTTCTTTGTTTGGGTCATCATCTTATTCCAAAAGGCCTTTTC |
| | SARS-CoV-1- | CATGCCTTTGGGTGTTGTGACTAACAGCACTTTAGAAGTAACAGAGA |
| | RBD | TTGACCAGCTAGTCTGCAAGGATCATCTTGCATCTACTGACCAGCTG |
| | | AAATCAGTTGGTCTCAACCTCGAGGGGAGCGGAGTATCTACTGATAT |
| | | CCCATCTGCAACAAAGCGTTGGGGCTTCAGATCTGGTGTTCCTCCCA |
| | | AGGTGGTCAGCTATGAAGCGGGAGAATGGGCTGAAAATTGCTACAA |
| | | TCTTGAAATAAAGAAGCCGGACGGGAGCGAATGCTTACCCCCACCG |
| | | CCAGATGGTGTCAGAGGCTTTCCAAGGTGCCGCTATGTTCACAAAGC |
| | | CCAAGGAACCGGGCCCTGCCCAGGTGACTACGCCTTTCACAAGGAT |
| | | GGAGCTTTCTTCCTCTATGACAGGCTGGCTTCAACTGTAATTTACAG |
| | | AGGAGTCAATTTTGCTGAGGGGGTAATTGCATTCTTGATATTGGCTA |
| | | AACCAAAAGAAACGTTCCTTCAGTCACCCCCCATTCGAGAGGCAGT |
| | | AAACTACACTGAAAATACATCAAGTTATTATGCCACATCCTACTTGG |
| | | AGTATGAAATCGAAAATTTTGGTGCTCAACACTCCACGACCCTTTTC |
| | | AAAATTGACAATAATACTTTTGTTCGTCTGGACAGGCCCCACACGCC |
| | | TCAGTTCCTTTTCCAGCTGAATGATACCATTCACCTTCACCAACAGTT |
| | | GAGTAATACAACTGGGAGACTAATTTGGACACTAGATGCTAATATC |
| | | AATGCTGATATTGGTGAATGGGCTTTTTGGGAAAATAAAAAGAATCT |
| | | CTCCGAACAACTACGTGGAGAAGAGCTGTCTTTCGAAGCTTTATCGC |
| | | TCCGCTTCCCAAATATCACCAACCTGTGCCCCTTCGGCGAGGTGTTT |
| | | AATGCCACAAAGTTCCCCAGCGTGTACGCCTGGGAGCGCAAGAAGA |
| | | TCAGCAACTGCGTGGCCGACTACTCCGTGCTGTATAATAGCACCTTC |
| | | TTCAGCACCTTCAAGTGCTACGGCGTGAGCGCCACCAAGCTGAATG |
| | | ACCTGTGCTTCTCTAACGTGTATGCCGATAGCTTTGTGGTGAAGGGC |
| | | GACGATGTGAGGCAGATCGCACCTGGACAGACCGGCGTGATCGCAG |
| | | ACTACAACTATAAGCTGCCAGACGATTTCATGGGCTGCGTGCTGGCC |
| | | TGGAATACACGCAACATCGATGCCACATCCACCGGCAACTACAATT |
| | | ATAAGTACCGGTATCTGAGACACGGCAAGCTGCGGCCCTTCGAGAG |
| | | AGACATCTCCAATGTGCCATTTTCTCCAGATGGCAAGCCATGCACCC |
| | | CACCTGCCCTGAATTGTTACTGGCCTCTGAACGACTACGGCTTCTAT |
| | | ACCACAACCGGCATCGGCTACCAGCCTTATAGGGTGGTGGTGCTGTC |
| | | CTTTGAGCTGCTGAACGCACCTGCAACCGTGTGCGGACCAAAGCTGT |
| | | CTACCGACCTGATCAAGAATCAGAATATAACAACAGCGGTTAAAAC |
| | | TGTCCTGCCACAGGAGTCCACAAGCAACGGTCTAATAACTTCAACA |
| | | GTAACAGGGATTCTTGGGAGTCTTGGGCTTCGAAAACGCAGCAGAA |
| | | GACAAACTAACACCAAAGCCACGGGTAAGTGCAATCCCAACTTACA |
| | | CTACTGGACTGCACAAGAACAACATAATGCTGCTGGGATTGCCTGG |
| | | ATCCCGTACTTTGGACCGGGTGCGGAAGGCATATACACTGAAGGCC |
| | | TGATGCATAACCAAAATGCCTTAGTCTGTGGACTTAGGCAACTTGCA |
| | | AATGAAACAACTCAAGCTCTGCAGCTTTTCTTAAGAGCCACAACGG |
| | | AGCTGCGGACATATACCATACTCAATAGGAAGGCCATAGATTTCCTT |
| | | CTGCGACGATGGGGGGGGACATGCAGGATCCTGGGACCAGATTGTT |
| | | GCATTGAGCCACATGATTGGACAAAAAACATCACTGATAAAATCAA |
| | | CCAAATCATCCATGACTTCATCGACAACCCCTTACCTAATCAGGATA |
| | | ATGATGATAATTGGTGGACGGGCTGGAGACAGTGGATCCCTGCAGG |
| | | AATAGGCATTACTGGAATTATTATTGCAATTATTGCTCTTCTTTGCGT |
| | | TTGCAAGCTGCTTTGCTGA |
| 4 | BDBV-GP- | ATGGTGACCAGCGGCATCCTGCAGCTGCCCCGGGAGCGGTTCAGAA |
| | deltaMLD- | AGACCAGCTTCTTCGTGTGGGTGATCATCCTGTTCCACAAGGTGTTC |
| | MERS-CoV- | CCCATCCCCCTGGGAGTCGTCCACAACAACACCCTGCAGGTCTCCGA |
| | RBD | CATCGACAAGCTGGTGTGCCGGGACAAACTGTCATCCACAAGCCAG |
| | | CTGAAGTCCGTGGGCCTGAACCTGGAAGGCAACGGAGTCGCTACAG |
| | | ATGTCCCCACCGCCACCAAGAGATGGGGCTTCAGAGCCGGCGTGCC |
| | | CCCCAAGGTGGTGAACTACGAGGCCGGCGAGTGGGCCGAGAACTGC |
| | | TACAACCTGGACATCAAGAAGGCCGACGGCAGCGAGTGTCTGCCTG |
| | | AGGCCCCCGAGGGAGTGAGAGGCTTCCCCCGGTGCAGATACGTGCA |
| | | CAAGGTGTCCGGCACCGGCCCTTGCCCTGAGGGCTACGCCTTCCACA |
| | | AAGAGGGCGCCTTTTTCCTGTACGACAGACTGGCTTCCACCATCATC |
| | | TACAGAAGCACCACCTTCAGCGAAGGAGTCGTCGCCTTCCTGATCCT |
| | | GCCCGAGACAAAGAAGGACTTCTTCCAGAGCCCCCCTCTGCACGAG |
| | | CCCGCCAACATGACCACCGACCCCAGCAGCTACTACCACACCGTGA |
| | | CCCTGAATTACGTGGCCGACAACTTCGGCACCAATATGACCAACTTT |
| | | CTGTTCCAGGTGGACCATCTGACCTACGTGCAGCTGGAACCCCGGTT |
| | | CACCCCCCAGTTTCTGGTGCAGCTGAACGAGACAATCTACACCAACG |
| | | GCAGGCGGAGCAACACCACCGGCACCCTGATCTGGAAAGTGAACCC |
| | | CACCGTGGACACCGGCGTGGGCGAATGGGCCTTCTGGGAGAACAAG |
| | | AAGAACTTCACCAAGACCCTGAGCAGCGAGGAACTGAGCGTGATCT |
| | | TCGTGCCCAGACAGGCAGAGGGAGTGGAGTGCGACTTCTCCCCACT |
| | | GCTGTCTGGCACACCACCTCAGGTGTATAATTTCAAGAGGCTGGTGT |
| | | TTACAAACTGTAATTACAACCTGACCAAGCTGCTGTCCCTGTTCTCT |
| | | GTGAACGACTTCACCTGCTCCCAGATCAGCCCTGCCGCCATCGCCTC |
| | | CAATTGTTATAGCTCCCTGATCCTGGATTACTTCTCTTATCCCCTGTC |
| | | TATGAAGAGCGACCTGTCCGTGTCTAGCGCCGGCCCTATCAGCCAGT |
| | | TTAATTACAAGCAGTCCTTCTCTAACCCCACATGCCTGATCCTGGCC |
| | | ACCGTGCCTCACAACCTGACCACAATCACAAAGCCACTGAAGTACT |
| | | CCTATATCAATAAGTGCAGCAGGCTGCTGTCCGACGACCGCACCGA |
| | | GGTGCCTCAGCTGGTGAATGCCAACCAGTACTCTCCATGCGTGAGCA |
| | | TCGTGCCATCCACCGTGTGGGAGGACGGCGATTACTATAGAAAGCA |
| | | GCTGAGCCCACTGGAGGGAGGAGGATGGCTGGTGGCAAGCGGCTCC |
| | | ACAGTGGCCATGACCGAGCAGCTCCAGATGGGCTTCGGCATCACAG |
| | | TGCAGTACGGCACAGATACCAATAGCGTGTGCCCCAAGCTGGAGTT |
| | | TGCCAACGACACCAAGATCGCCTCCCAGACCGACAGCAACCGGCCC |
| | | AACCCCATCGACATCAGCGAGAGCACCGAGCCTGGACCTCTGACAA |
| | | ATACAACAAGAGGAGCTGCTAATCTGCTGACCGGCAGCCGGCGGAC |
| | | CAGACGGGAGATCACCCTGCGGACCCAGGCCAAGTGCAACCCCAAT |
| | | CTGCACTACTGGACCACCCAGGACGAGGGAGCCGCCATCGGCCTGG |
| | | CCTGGATCCCTTACTTCGGCCCTGCCGCCGAGGGCATCTACACAGAG |
| | | GGCATCATGCACAACCAGAACGGCCTGATCTGCGGCCTGCGGCAGC |
| | | TGGCCAACGAGACAACCCAGGCCCTGCAGCTGTTCCTGCGGGCCAC |
| | | CACCGAGCTGCGGACCTTCAGCATCCTGAACAGAAAGGCCATCGAC |
| | | TTTCTGCTGCAGCGCTGGGGCGGCACCTGTCACATCCTGGGCCCCGA |
| | | CTGCTGCATCGAGCCCCACGACTGGACCAAGAACATCACCGACAAG |
| | | ATCGACCAGATCATCCACGACTTCATCGATAAGCCCCTGCCCGACCA |
| | | GACCGACAACGACAATTGGTGGACAGGCTGGCGGCAGTGGGTGCCA |
| | | GCCGGCATCGGCATCACCGGCGTGATCATTGCCGTGATCGCCCTGCT |
| | | GTGCATCTGCAAGTTTCTGCTGTGA |
| 5 | ZEBOV-GP- | ATGGGCGTGACTGGAATCCTGCAGCTGCCAAGAGATCGGTTCAAGC |
| | deltaMLD- | GGACTTCATTTTTCCTGTGGGTCATCATCCTGTTTCAGAGAACCTTTT |
| | mCherry | CTATTCCTCTTGGCGTTATCCATAATAGTACTCTCCAAGTCAGCGAC |
| | | GTCGATAAACTTGTTTGCCGCGATAAACTTTCCAGTACTAACCAGCT |
| | | GCGCTCCGTGGGTCTTAACCTCGAAGGAAACGGAGTTGCCACCGAT |
| | | GTTCCCTCCGCCACTAAGCGCTGGGGCTTCCGCTCCGGAGTCCCACC |
| | | CAAGGTAGTGAACTATGAGGCGGGCGAATGGGCCGAGAACTGTTAT |
| | | AATCTTGAGATAAAAAAACCCGACGGATCCGAGTGCCTTCCAGCTG |
| | | CCCCGGATGGTATTAGGGGCTTCCCACGCTGTCGCTACGTTCATAAA |
| | | GTATCCGGAACCGGACCTTGTGCTGGAGACTTCGCATTCCACAAAGA |
| | | GGGCGCGTTTTTTCTGTATGATCGCCTTGCATCCACCGTAATCTACCG |
| | | CGGCACGACCTTTGCTGAAGGTGTGGTCGCTTTCCTTATTCTCCCAC |
| | | AAGCTAAGAAGGACTTTTTCTCCTCCCACCCACTCCGCGAGCCAGTA |
| | | AACGCAACTGAAGATCCCTCCAGCGGTTACTACTCCACCACCATTCG |
| | | CTACCAGGCAACCGGATTTGGAACAAATGAAACAGAGTACCTGTTC |
| | | GAAGTTGATAATCTGACATACGTCCAGCTTGAATCCCGCTTCACTCC |
| | | CCAGTTTTTGCTGCAACTCAACGAAACAATATACACATCCGGCAAGC |
| | | GCTCCAACACCACCGGCAAATTGATATGGAAAGTAAACCCTGAAAT |
| | | TGACACAACTATCGGAGAATGGGCATTCTGGGAAACAAAAAAAAAT |
| | | CTCACTCGCAAAATTAGGTCCGAGGAACTCAGCTTCACAGTAGTTTC |
| | | CAATGGCAGCGGAGTGAGCAAGGGCGAGGAGGATAACATGGCCAT |
| | | CATCAAGGAGTTCATGCGCTTCAAGGTGCACATGGAGGGCTCCGTG |
| | | AACGGCCACGAGTTCGAGATCGAGGGCGAGGGCGAGGGCCGCCCCT |
| | | ACGAGGGCACCCAGACCGCCAAGCTGAAGGTGACCAAGGGTGGCCC |
| | | CCTGCCCTTCGCCTGGGACATCCTGTCCCCTCAGTTCATGTACGGCTC |
| | | CAAGGCCTACGTGAAGCACCCCGCCGACATCCCCGACTACTTGAAG |
| | | CTGTCCTTCCCCGAGGGCTTCAAGTGGGAGCGCGTGATGAACTTCGA |
| | | GGACGGCGGCGTGGTGACCGTGACCCAGGACTCCTCCCTGCAGGAC |
| | | GGCGAGTTCATCTACAAGGTGAAGCTGCGCGGCACCAACTTCCCCTC |
| | | CGACGGCCCCGTAATGCAGAAGAAGACCATGGGCTGGGAGGCCTCC |
| | | TCCGAGCGGATGTACCCCGAGGACGGCGCCCTGAAGGGCGAGATCA |
| | | AGCAGAGGCTGAAGCTGAAGGACGGCGGCCACTACGACGCTGAGGT |
| | | CAAGACCACCTACAAGGCCAAGAAGCCCGTGCAGCTGCCCGGCGCC |
| | | TACAACGTCAACATCAAGTTGGACATCACCTCCCACAACGAGGACT |
| | | ACACCATCGTGGAACAGTACGAACGCGCCGAGGGCCGCCACTCCAC |
| | | CGGCGGCATGGACGAGCTGTACAAGGGCAGCGGAAACACCCACCAT |
| | | CAGGACACAGGAGAGGAATCCGCATCCTCCGGTAAACTTGGTTTGA |
| | | TAACAAACACCATCGCAGGCGTAGCTGGACTGATAACAGGCGGACG |
| | | CCGCACCAGGAGGGAAGCTATTGTCAACGCTCAACCAAAATGCAAC |
| | | CCCAACCTTCACTATTGGACCACTCAGGATGAAGGAGCCGCTATAG |
| | | GACTCGCATGGATACCTTACTTCGGTCCCGCAGCTGAAGGCATATAC |
| | | ACCGAAGGACTGATGCATAACCAAGACGGTCTGATTTGTGGTCTTAG |
| | | GCAGCTGGCAAACGAAACCACTCAGGCCCTGCAGCTGTTCTTGCGC |
| | | GCTACGACAGAACTCCGCACCTTTAGTATCTTGAACCGCAAAGCTAT |
| | | TGATTTCCTTCTTCAACGCTGGGGCGGCACCTGCCACATCCTGGGAC |
| | | CCGATTGCTGTATTGAACCCCACGACTGGACCAAAAACATCACAGA |
| | | CAAGATAGATCAGATAATCCACGATTTTGTAGACAAGACCCTCCCA |
| | | GACCAAGGTGACAATGACAATTGGTGGACAGGTTGGCGCCAATGGA |
| | | TCCCTGCAGGCATTGGTGTAACAGGAGTAATCATAGCCGTTATCGCC |
| | | CTGTTTTGTATTTGTAAATTCGTGTTTTGA |
| 6 | pVenus- | ACGAAGACAAACAAACCATTATTATCATTAAAAGGCTCAGGAGAAA |
| | ZEBOV-GP- | CTTTAACAGTAATCAGAATTCTCGAGGATCCACCGGTCGCCACCATG |
| | deltaMLD- | GTGAGCAAGGGCGAGGAGCTGTTCACCGGGGTGGTGCCCATCCTGG |
| | SARS-CoV-2- | TCGAGCTGGACGGCGACGTAAACGGCCACAAGTTCAGCGTGTCCGG |
| | RBD | CGAGGGCGAGGGCGATGCCACCTACGGCAAGCTGACCCTGAAGTTA |
| | | ATCTGCACCACCGGCAAGCTGCCCGTGCCCTGGCCCACCCTCGTGAC |
| | | CACCTTAGGCTACGGCCTGCAGTGCTTCGCCCGCTACCCCGACCACA |
| | | TGAAGCAGCACGACTTCTTCAAGTCCGCCATGCCCGAAGGCTACGTC |
| | | CAGGAGCGCACCATCTTCTTCAAGGACGACGGCAACTACAAGACCC |
| | | GCGCCGAGGTGAAGTTCGAGGGCGACACCCTGGTGAACCGCATCGA |
| | | GCTGAAGGGCATCGACTTCAAGGAGGACGGCAACATCCTGGGGCAC |
| | | AAGCTGGAGTACAACTACAACAGCCACAACGTCTATATCACGGCCG |
| | | ACAAGCAGAAGAACGGCATCAAGGCGAACTTCAAGATCCGCCACAA |
| | | CATCGAGGACGGCGGCGTGCAGCTCGCCGACCACTACCAGCAGAAC |
| | | ACCCCCATCGGCGACGGCCCCGTGCTGCTGCCCGACAACCACTACCT |
| | | GAGCTACCAGTCCGCCCTGAGCAAAGACCCCAACGAGAAGCGCGAT |
| | | CACATGGTCCTGCTGGAGTTCGTGACCGCCGCCGGGATCACTCTCGG |
| | | CATGGACGAGCTGTACAAGTAAAGCGGCCGCCCTCCTCAGGTGCTG |
| | | GCCATATGAAAAAAACTAACAGTAATCAAAATGTCTGTTACAGTCA |
| | | AGAGAATCATTGACAACACAGTCATAGTTCCAAAACTTCCTGCAAAT |
| | | GAGGATCCAGTGGAATACCCGGCAGATTACTTCAGAAAATCAAAGG |
| | | AGATTCCTCTTTACATCAATACTACAAAAAGTTTGTCAGATCTAAGA |
| | | GGATATGTCTACCAAGGCCTCAAATCCGGAAATGTATCAATCATACA |
| | | TGTCAACAGCTACTTGTATGGAGCATTAAAGGACATCCGGGGTAAG |
| | | TTGGATAAAGATTGGTCAAGTTTCGGAATAAACATCGGGAAAGCAG |
| | | GGGATACAATCGGAATATTTGACCTTGTATCCTTGAAAGCCCTGGAC |
| | | GGCGTACTTCCAGATGGAGTATCGGATGCTTCCAGAACCAGCGCAG |
| | | ATGACAAATGGTTGCCTTTGTATCTACTTGGCTTATACAGAGTGGGC |
| | | AGAACACAAATGCCTGAATACAGAAAAAAGCTCATGGATGGGCTGA |
| | | CAAATCAATGCAAAATGATCAATGAACAGTTTGAACCTCTTGTGCCA |
| | | GAAGGTCGTGACATTTTTGATGTGTGGGGAAATGACAGTAATTACAC |
| | | AAAAATTGTCGCTGCAGTGGACATGTTCTTCCACATGTTCAAAAAAC |
| | | ATGAATGTGCCTCGTTCAGATACGGAACTATTGTTTCCAGATTCAAA |
| | | GATTGTGCTGCATTGGCAACATTTGGACACCTCTGCAAAATAACCGG |
| | | AATGTCTACAGAAGATGTAACGACCTGGATCTTGAACCGAGAAGTT |
| | | GCAGATGAAATGGTCCAAATGATGCTTCCAGGCCAAGAAATTGACA |
| | | AGGCCGATTCATACATGCCTTATTTGATCGACTTTGGATTGTCTTCTA |
| | | AGTCTCCATATTCTTCCGTCAAAAACCCTGCCTTCCACTTCTGGGGG |
| | | CAATTGACAGCTCTTCTGCTCAGATCCACCAGAGCAAGGAATGCCCG |
| | | ACAGCCTGATGACATTGAGTATACATCTCTTACTACAGCAGGTTTGT |
| | | TGTACGCTTATGCAGTAGGATCCTCTGCCGACTTGGCACAACAGTTT |
| | | TGTGTTGGAGATAACAAATACACTCCAGATGATAGTACCGGAGGAT |
| | | TGACGACTAATGCACCGCCACAAGGCAGAGATGTGGTCGAATGGCT |
| | | CGGATGGTTTGAAGATCAAAACAGAAAACCGACTCCTGATATGATG |
| | | CAGTATGCGAAAAGAGCAGTCATGTCACTGCAAGGCCTAAGAGAGA |
| | | AGACAATTGGCAAGTATGCTAAGTCAGAATTTGACAAATGACCCTA |
| | | TAATTCTCAGATCACCTATTATATATTATGCTACATATGAAAAAAAC |
| | | TAACAGATATCATGGATAATCTCACAAAAGTTCGTGAGTATCTCAAG |
| | | TCCTACTCTCGTCTAGATCAGGCGGTAGGAGAGATAGATGAGATCG |
| | | AAGCACAACGAGCTGAAAAGTCCAATTATGAGTTGTTCCAAGAGGA |
| | | CGGAGTGGAAGAGCATACTAGGCCCTCTTATTTTCAGGCAGCAGAT |
| | | GATTCTGACACAGAATCTGAACCAGAAATTGAAGACAATCAAGGCT |
| | | TGTATGTACCAGATCCGGAAGCTGAGCAAGTTGAAGGCTTTATACA |
| | | GGGGCCTTTAGATGACTATGCAGATGAGGACGTGGATGTTGTATTCA |
| | | CTTCGGACTGGAAACAGCCTGAGCTTGAATCCGACGAGCATGGAAA |
| | | GACCTTACGGTTGACATTGCCAGAGGGTTTAAGTGGAGAGCAGAAA |
| | | TCCCAGTGGCTTTTGACGATTAAAGCAGTCGTTCAAAGTGCCAAACA |
| | | CTGGAATCTGGCAGAGTGCACATTTGAAGCATCGGGAGAAGGGGTC |
| | | ATCATAAAAAAGCGCCAGATAACTCCGGATGTATATAAGGTCACTC |
| | | CAGTGATGAACACACATCCGTACCAATCAGAAGCCGTATCAGATGT |
| | | TTGGTCTCTCTCAAAGACATCCATGACTTTCCAACCCAAGAAAGCAA |
| | | GTCTTCAGCCTCTCACCATATCCTTGGATGAATTGTTCTCATCTAGAG |
| | | GAGAATTCATCTCTGTCGGAGGTAACGGACGAATGTCTCATAAAGA |
| | | GGCCATCCTGCTCGGTCTGAGGTACAAAAAGTTGTACAATCAGGCG |
| | | AGAGTCAAATATTCTCTGTAGACTATGAAAAAAAGTAACAGATATC |
| | | ACAATCTAAGTGTTATCCCAATCCATTCATCATGAGTTCCTTAAAGA |
| | | AGATTCTCGGTCTGAAGGGGAAAGGTAAGAAATCTAAGAAATTAGG |
| | | GATCGCACCACCCCCTTATGAAGAGGACACTAGCATGGAGTATGCT |
| | | CCGAGCGCTCCAATTGACAAATCCTATTTTGGAGTTGACGAGATGGA |
| | | CACCTATGATCCGAATCAATTAAGATATGAGAAATTCTTCTTTACAG |
| | | TGAAAATGACGGTTAGATCTAATCGTCCGTTCAGAACATACTCAGAT |
| | | GTGGCAGCCGCTGTATCCCATTGGGATCACATGTACATCGGAATGGC |
| | | AGGGAAACGTCCCTTCTACAAAATCTTGGCTTTTTTGGGTTCTTCTAA |
| | | TCTAAAGGCCACTCCAGCGGTATTGGCAGATCAAGGTCAACCAGAG |
| | | TATCACGCTCACTGCGAAGGCAGGGCTTATTTGCCACATAGGATGGG |
| | | GAAGACCCCTCCCATGCTCAATGTACCAGAGCACTTCAGAAGACCA |
| | | TTCAATATAGGTCTTTACAAGGGAACGATTGAGCTCACAATGACCAT |
| | | CTACGATGATGAGTCACTGGAAGCAGCTCCTATGATCTGGGATCATT |
| | | TCAATTCTTCCAAATTTTCTGATTTCAGAGAGAAGGCCTTAATGTTTG |
| | | GCCTGATTGTCGAGAAAAAGGCATCTGGAGCGTGGGTCCTGGATTCT |
| | | ATCAGCCACTTCAAATGAGCTAGTCTAGCTTCCAGCTTCTGAACAAT |
| | | CCCCGGTTTACTCAGTCTCTCCTAATTCCAGCCTTTCGAACAACTAAT |
| | | ATCCTGTCTTTTCTATCCCTATGAAAAAAACTAACAGAGATCGATCT |
| | | GTTTCCTTGACACGCGTGGCCCAGGCGGCCCACGATGGGCGTGACTG |
| | | GAATCCTGCAGCTGCCAAGAGATCGGTTCAAGCGGACTTCATTTTTC |
| | | CTGTGGGTCATCATCCTGTTTCAGAGAACCTTTTCTATTCCTCTTGGC |
| | | GTTATCCATAATAGTACTCTCCAAGTCAGCGACGTCGATAAACTTGT |
| | | TTGCCGCGATAAACTTTCCAGTACTAACCAGCTGCGCTCCGTGGGTC |
| | | TTAACCTCGAAGGAAACGGAGTTGCCACCGATGTTCCCTCCGCCACT |
| | | AAGCGCTGGGGCTTCCGCTCCGGAGTCCCACCCAAGGTAGTGAACT |
| | | ATGAGGCGGGCGAATGGGCCGAGAACTGTTATAATCTTGAGATAAA |
| | | AAAACCCGACGGATCCGAGTGCCTTCCAGCTGCCCCGGATGGTATTA |
| | | GGGGCTTCCCACGCTGTCGCTACGTTCATAAAGTATCCGGAACCGGA |
| | | CCTTGTGCTGGAGACTTCGCATTCCACAAAGAGGGCGCGTTTTTTCT |
| | | GTATGATCGCCTTGCATCCACCGTAATCTACCGCGGCACGACCTTTG |
| | | CTGAAGGTGTGGTCGCTTTCCTTATTCTCCCACAAGCTAAGAAGGAC |
| | | TTTTTCTCCTCCCACCCACTCCGCGAGCCAGTAAACGCAACTGAAGA |
| | | TCCCTCCAGCGGTTACTACTCCACCACCATTCGCTACCAGGCAACCG |
| | | GATTTGGAACAAATGAAACAGAGTACCTGTTCGAAGTTGATAATCT |
| | | GACATACGTCCAGCTTGAATCCCGCTTCACTCCCCAGTTTTTGCTGC |
| | | AACTCAACGAAACAATATACACATCCGGCAAGCGCTCCAACACCAC |
| | | CGGCAAATTGATATGGAAAGTAAACCCTGAAATTGACACAACTATC |
| | | GGAGAATGGGCATTCTGGGAAACAAAAAAAAATCTCACTCGCAAAA |
| | | TTAGGTCCGAGGAACTCAGCTTCACAGTAGTTTCCAATCGCTTTCCT |
| | | AATATCACAAACCTGTGCCCATTTGGCGAGGTGTTCAACGCAACCCG |
| | | CTTCGCCAGCGTGTACGCCTGGAATAGGAAGCGGATCAGCAACTGC |
| | | GTGGCCGACTATAGCGTGCTGTACAACTCCGCCTCTTTCAGCACCTT |
| | | TAAGTGCTATGGCGTGTCCCCCACAAAGCTGAATGACCTGTGCTTTA |
| | | CCAACGTCTACGCCGATTCTTTCGTGATCAGGGGCGACGAGGTGCGC |
| | | CAGATCGCCCCCGGCCAGACAGGCAAGATCGCAGACTACAATTATA |
| | | AGCTGCCAGACGATTTCACCGGCTGCGTGATCGCCTGGAACAGCAA |
| | | CAATCTGGATTCCAAAGTGGGCGGCAACTACAATTATCTGTACCGGC |
| | | TGTTTAGAAAGAGCAATCTGAAGCCCTTCGAGAGGGACATCTCTAC |
| | | AGAAATCTACCAGGCCGGCAGCACCCCTTGCAATGGCGTGGAGGGC |
| | | TTTAACTGTTATTTCCCACTCCAGTCCTACGGCTTCCAGCCCACAAAC |
| | | GGCGTGGGCTATCAGCCTTACCGCGTGGTGGTGCTGAGCTTTGAGCT |
| | | GCTGCACGCCCCAGCAACAGTGTGCGGCCCCAAGAAGTCCACCAAT |
| | | CTGGTGAAGAACACCCACCATCAGGACACAGGAGAGGAATCCGCAT |
| | | CCTCCGGTAAACTTGGTTTGATAACAAACACCATCGCAGGCGTAGCT |
| | | GGACTGATAACAGGCGGACGCCGCACCAGGAGGGAAGCTATTGTCA |
| | | ACGCTCAACCAAAATGCAACCCCAACCTTCACTATTGGACCACTCAG |
| | | GATGAAGGAGCCGCTATAGGACTCGCATGGATACCTTACTTCGGTCC |
| | | CGCAGCTGAAGGCATATACACCGAAGGACTGATGCATAACCAAGAC |
| | | GGTCTGATTTGTGGTCTTAGGCAGCTGGCAAACGAAACCACTCAGGC |
| | | CCTGCAGCTGTTCTTGCGCGCTACGACAGAACTCCGCACCTTTAGTA |
| | | TCTTGAACCGCAAAGCTATTGATTTCCTTCTTCAACGCTGGGGCGGC |
| | | ACCTGCCACATCCTGGGACCCGATTGCTGTATTGAACCCCACGACTG |
| | | GACCAAAAACATCACAGACAAGATAGATCAGATAATCCACGATTTT |
| | | GTAGACAAGACCCTCCCAGACCAAGGTGACAATGACAATTGGTGGA |
| | | CAGGTTGGCGCCAATGGATCCCTGCAGGCATTGGTGTAACAGGAGT |
| | | AATCATAGCCGTTATCGCCCTGTTTTGTATTTGTAAATTCGTGTTTTG |
| | | AGGCCCTCGCGGCCGCCCTGCACAACAGATTCTTCATGITTGAACCA |
| | | AATCAACTTGTGATATCATGCTCAAAGAGGCCTTAATTATATTTTAA |
| | | TTTTTAATTTTTATGAAAAAAACTAACAGCAATCATGGAAGTCCACG |
| | | ATTTTGAGACCGACGAGTTCAATGATTTCAATGAAGATGACTATGCC |
| | | ACAAGAGAATTCCTGAATCCCGATGAGCGCATGACGTACTTGAATC |
| | | ATGCTGATTACAATTTGAATTCTCCTCTAATTAGTGATGATATTGAC |
| | | AATTTGATCAGGAAATTCAATTCTCTTCCGATTCCCTCGATGTGGGA |
| | | TAGTAAGAACTGGGATGGAGTTCTTGAGATGTTAACATCATGTCAAG |
| | | CCAATCCCATCTCAACATCTCAGATGCATAAATGGATGGGAAGTTGG |
| | | TTAATGTCTGATAATCATGATGCCAGTCAAGGGTATAGTTTTTTACA |
| | | TGAAGTGGACAAAGAGGCAGAAATAACATTTGACGTGGTGGAGACC |
| | | TTCATCCGCGGCTGGGGCAACAAACCAATTGAATACATCAAAAAGG |
| | | AAAGATGGACTGACTCATTCAAAATTCTCGCTTATTTGTGTCAAAAG |
| | | TTTTTGGACTTACACAAGTTGACATTAATCTTAAATGCTGTCTCTGAG |
| | | GTGGAATTGCTCAACTTGGCGAGGACTTTCAAAGGCAAAGTCAGAA |
| | | GAAGTTCTCATGGAACGAACATATGCAGGATTAGGGTTCCCAGCTTG |
| | | GGTCCTACTTTTATTTCAGAAGGATGGGCTTACTTCAAGAAACTTGA |
| | | TATTCTAATGGACCGAAACTTTCTGTTAATGGTCAAAGATGTGATTA |
| | | TAGGGAGGATGCAAACGGTGCTATCCATGGTATGTAGAATAGACAA |
| | | CCTGTTCTCAGAGCAAGACATCTTCTCCCTTCTAAATATCTACAGAA |
| | | TTGGAGATAAAATTGTGGAGAGGCAGGGAAATTTTTCTTATGACTTG |
| | | ATTAAAATGGTGGAACCGATATGCAACTTGAAGCTGATGAAATTAG |
| | | CAAGAGAATCAAGGCCTTTAGTCCCACAATTCCCTCATTTTGAAAAT |
| | | CATATCAAGACTTCTGTTGATGAAGGGGCAAAAATTGACCGAGGTA |
| | | TAAGATTCCTCCATGATCAGATAATGAGTGTGAAAACAGTGGATCTC |
| | | ACACTGGTGATTTATGGATCGTTCAGACATTGGGGTCATCCTTTTAT |
| | | AGATTATTACACTGGACTAGAAAAATTACATTCCCAAGTAACCATGA |
| | | AGAAAGATATTGATGTGTCATATGCAAAAGCACTTGCAAGTGATTTA |
| | | GCTCGGATTGTTCTATTTCAACAGTTCAATGATCATAAAAAGTGGTT |
| | | CGTGAATGGAGACTTGCTCCCTCATGATCATCCCTTTAAAAGTCATG |
| | | TTAAAGAAAATACATGGCCCACAGCTGCTCAAGTTCAAGATTTTGGA |
| | | GATAAATGGCATGAACTTCCGCTGATTAAATGTTTTGAAATACCCGA |
| | | CTTACTAGACCCATCGATAATATACTCTGACAAAAGTCATTCAATGA |
| | | ATAGGTCAGAGGTGTTGAAACATGTCCGAATGAATCCGAACACTCC |
| | | TATCCCTAGTAAAAAGGTGTTGCAGACTATGTTGGACACAAAGGCT |
| | | ACCAATTGGAAAGAATTTCTTAAAGAGATTGATGAGAAGGGCTTAG |
| | | ATGATGATGATCTAATTATTGGTCTTAAAGGAAAGGAGAGGGAACT |
| | | GAAGTTGGCAGGTAGATTTTTCTCCCTAATGTCTTGGAAATTGCGAG |
| | | AATACTTTGTAATTACCGAATATTTGATAAAGACTCATTTCGTCCCT |
| | | ATGTTTAAAGGCCTGACAATGGCGGACGATCTAACTGCAGTCATTAA |
| | | AAAGATGTTAGATTCCTCATCCGGCCAAGGATTGAAGTCATATGAG |
| | | GCAATTTGCATAGCCAATCACATTGATTACGAAAAATGGAATAACC |
| | | ACCAAAGGAAGTTATCAAACGGCCCAGTGTTCCGAGTTATGGGCCA |
| | | GTTCTTAGGTTATCCATCCTTAATCGAGAGAACTCATGAATTTTTTGA |
| | | GAAAAGTCTTATATACTACAATGGAAGACCAGACTTGATGCGTGTTC |
| | | ACAACAACACACTGATCAATTCAACCTCCCAACGAGTTTGTTGGCAA |
| | | GGACAAGAGGGTGGACTGGAAGGTCTACGGCAAAAAGGATGGAGT |
| | | ATCCTCAATCTACTGGTTATTCAAAGAGAGGCTAAAATCAGAAACA |
| | | CTGCTGTCAAAGTCTTGGCACAAGGTGATAATCAAGTTATTTGCACA |
| | | CAGTATAAAACGAAGAAATCGAGAAACGTTGTAGAATTACAGGGTG |
| | | CTCTCAATCAAATGGTTTCTAATAATGAGAAAATTATGACTGCAATC |
| | | AAAATAGGGACAGGGAAGTTAGGACTTTTGATAAATGACGATGAGA |
| | | CTATGCAATCTGCAGATTACTTGAATTATGGAAAAATACCGATTTTC |
| | | CGTGGAGTGATTAGAGGGTTAGAGACCAAGAGATGGTCACGAGTGA |
| | | CTTGTGTCACCAATGACCAAATACCCACTTGTGCTAATATAATGAGC |
| | | TCAGTTTCCACAAATGCTCTCACCGTAGCTCATTTTGCTGAGAACCC |
| | | AATCAATGCCATGATACAGTACAATTATTTTGGGACATTTGCTAGAC |
| | | TCTTGTTGATGATGCATGATCCTGCTCTTCGTCAATCATTGTATGAAG |
| | | TTCAAGATAAGATACCGGGCTTGCACAGTTCTACTTTCAAATACGCC |
| | | ATGTTGTATTTGGACCCTTCCATTGGAGGAGTGTCGGGCATGTCTTT |
| | | GTCCAGGTTTTTGATTAGAGCCTTCCCAGATCCCGTAACAGAAAGTC |
| | | TCTCATTCTGGAGATTCATCCATGTACATGCTCGAAGTGAGCATCTG |
| | | AAGGAGATGAGTGCAGTATTTGGAAACCCCGAGATAGCCAAGTTTC |
| | | GAATAACTCACATAGACAAGCTAGTAGAAGATCCAACCTCTCTGAA |
| | | CATCGCTATGGGAATGAGTCCAGCGAACTTGTTAAAGACTGAGGTT |
| | | AAAAAATGCTTAATCGAATCAAGACAAACCATCAGGAACCAGGTGA |
| | | TTAAGGATGCAACCATATATTTGTATCATGAAGAGGATCGGCTCAGA |
| | | AGTTTCTTATGGTCAATAAATCCTCTGTTCCCTAGATTTTTAAGTGAA |
| | | TTCAAATCAGGCACTTTTTTGGGAGTCGCAGACGGGCTCATCAGTCT |
| | | ATTTCAAAATTCTCGTACTATTCGGAACTCCTTTAAGAAAAAGTATC |
| | | ATAGGGAATTGGATGATTTGATTGTGAGGAGTGAGGTATCCTCTTTG |
| | | ACACATTTAGGGAAACTTCATTTGAGAAGGGGATCATGTAAAATGT |
| | | GGACATGTTCAGCTACTCATGCTGACACATTAAGATACAAATCCTGG |
| | | GGCCGTACAGTTATTGGGACAACTGTACCCCATCCATTAGAAATGTT |
| | | GGGTCCACAACATCGAAAAGAGACTCCTTGTGCACCATGTAACACA |
| | | TCAGGGTTCAATTATGTTTCTGTGCATTGTCCAGACGGGATCCATGA |
| | | CGTCTTTAGTTCACGGGGACCATTGCCTGCTTATCTAGGGTCTAAAA |
| | | CATCTGAATCTACATCTATTTTGCAGCCTTGGGAAAGGGAAAGCAAA |
| | | GTCCCACTGATTAAAAGAGCTACACGTCTTAGAGATGCTATCTCTTG |
| | | GTTTGTTGAACCCGACTCTAAACTAGCAATGACTATACTTTCTAACA |
| | | TCCACTCTTTAACAGGCGAAGAATGGACCAAAAGGCAGCATGGGTT |
| | | CAAAAGAACAGGGTCTGCCCTTCATAGGTTTTCGACATCTCGGATGA |
| | | GCCATGGTGGGTTCGCATCTCAGAGCACTGCAGCATTGACCAGGTTG |
| | | ATGGCAACTACAGACACCATGAGGGATCTGGGAGATCAGAATTTCG |
| | | ACTTTTTATTCCAAGCAACGTTGCTCTATGCTCAAATTACCACCACTG |
| | | TTGCAAGAGACGGATGGATCACCAGTTGTACAGATCATTATCATATT |
| | | GCCTGTAAGTCCTGTTTGAGACCCATAGAAGAGATCACCCTGGACTC |
| | | AAGTATGGACTACACGCCCCCAGATGTATCCCATGTGCTGAAGACAT |
| | | GGAGGAATGGGGAAGGTTCGTGGGGACAAGAGATAAAACAGATCT |
| | | ATCCTTTAGAAGGGAATTGGAAGAATTTAGCACCTGCTGAGCAATCC |
| | | TATCAAGTCGGCAGATGTATAGGTTTTCTATATGGAGACTTGGCGTA |
| | | TAGAAAATCTACTCATGCCGAGGACAGTTCTCTATTTCCTCTATCTAT |
| | | ACAAGGTCGTATTAGAGGTCGAGGTTTCTTAAAAGGGTTGCTAGAC |
| | | GGATTAATGAGAGCAAGTTGCTGCCAAGTAATACACCGGAGAAGTC |
| | | TGGCTCATTTGAAGAGGCCGGCCAACGCAGTGTACGGAGGTTTGATT |
| | | TACTTGATTGATAAATTGAGTGTATCACCTCCATTCCTTTCTCTTACT |
| | | AGATCAGGACCTATTAGAGACGAATTAGAAACGATTCCCCACAAGA |
| | | TCCCAACCTCCTATCCGACAAGCAACCGTGATATGGGGGTGATTGTC |
| | | AGAAATTACTTCAAATACCAATGCCGTCTAATTGAAAAGGGAAAAT |
| | | ACAGATCACATTATTCACAATTATGGTTATTCTCAGATGTCTTATCCA |
| | | TAGACTTCATTGGACCATTCTCTATTTCCACCACCCTCTTGCAAATCC |
| | | TATACAAGCCATTTTTATCTGGGAAAGATAAGAATGAGTTGAGAGA |
| | | GCTGGCAAATCTTTCTTCATTGCTAAGATCAGGAGAGGGGTGGGAA |
| | | GACATACATGTGAAATTCTTCACCAAGGACATATTATTGTGTCCAGA |
| | | GGAAATCAGACATGCTTGCAAGTTCGGGATTGCTAAGGATAATAAT |
| | | AAAGACATGAGCTATCCCCCTTGGGGAAGGGAATCCAGAGGGACAA |
| | | TTACAACAATCCCTGTTTATTATACGACCACCCCTTACCCAAAGATG |
| | | CTAGAGATGCCTCCAAGAATCCAAAATCCCCTGCTGTCCGGAATCAG |
| | | GTTGGGCCAATTACCAACTGGCGCTCATTATAAAATTCGGAGTATAT |
| | | TACATGGAATGGGAATCCATTACAGGGACTTCTTGAGTTGTGGAGAC |
| | | GGCTCCGGAGGGATGACTGCTGCATTACTACGAGAAAATGTGCATA |
| | | GCAGAGGAATATTCAATAGTCTGTTAGAATTATCAGGGTCAGTCATG |
| | | CGAGGCGCCTCTCCTGAGCCCCCCAGTGCCCTAGAAACTTTAGGAGG |
| | | AGATAAATCGAGATGTGTAAATGGTGAAACATGTTGGGAATATCCA |
| | | TCTGACTTATGTGACCCAAGGACTTGGGACTATTTCCTCCGACTCAA |
| | | AGCAGGCTTGGGGCTTCAAATTGATTTAATTGTAATGGATATGGAAG |
| | | TTCGGGATTCTTCTACTAGCCTGAAAATTGAGACGAATGTTAGAAAT |
| | | TATGTGCACCGGATTTTGGATGAGCAAGGAGTTTTAATCTACAAGAC |
| | | TTATGGAACATATATTTGTGAGAGCGAAAAGAATGCAGTAACAATC |
| | | CTTGGTCCCATGTTCAAGACGGTCGACTTAGTTCAAACAGAATTTAG |
| | | TAGTTCTCAAACGTCTGAAGTATATATGGTATGTAAAGGTTTGAAGA |
| | | AATTAATCGATGAACCCAATCCCGATTGGTCTTCCATCAATGAATCC |
| | | TGGAAAAACCTGTACGCATTCCAGTCATCAGAACAGGAATTTGCCA |
| | | GAGCAAAGAAGGTTAGTACATACTTTACCTTGACAGGTATTCCCTCC |
| | | CAATTCATTCCTGATCCTTTTGTAAACATTGAGACTATGCTACAAAT |
| | | ATTCGGAGTACCCACGGGTGTGTCTCATGCGGCTGCCTTAAAATCAT |
| | | CTGATAGACCTGCAGATTTATTGACCATTAGCCTTTTTTATATGGCG |
| | | ATTATATCGTATTATAACATCAATCATATCAGAGTAGGACCGATACC |
| | | TCCGAACCCCCCATCAGATGGAATTGCACAAAATGTGGGGATCGCT |
| | | ATAACTGGTATAAGCTTTTGGCTGAGTTTGATGGAGAAAGACATTCC |
| | | ACTATATCAACAGTGTTTGGCAGTTATCCAGCAATCATTTCCGATTA |
| | | GGTGGGAGGCTATTTCAGTAAAAGGAGGATACAAGCAGAAGTGGAG |
| | | TACTAGAGGTGATGGGCTCCCAAAAGATACCCGAATTTCAGACTCCT |
| | | TGGCCCCAATCGGGAACTGGATCAGATCTTTGGAATTGGTCCGAAAC |
| | | CAAGTTCGTCTAAATCCATTCAATAAGATCTTGTTCAATCAGCTATG |
| | | TCGTACAGTGGATAATCATTTGAAGTGGTCAAATTTGCGAAAAAAC |
| | | ACAGGAATGATTGAATGGATCAATGGGCGAATTTCAAAAGAAGACC |
| | | GGTCTATACTGATGTTGAAGAGTGACCTACATGAGGAAAACTCTTGG |
| | | AGAGATTAAAAAATCAGGAGGAGACTCCAAACTTTAAGTATGAAAA |
| | | AAACTTTGATCCTTAAGACCCTCTTGTGGTTTTTATTTTTTTATCTGG |
| | | TTTTGTGGTCTTCGTGGGTCGGCATGGCATCTCCACCTCCTCGCGGTC |
| | | CGACCTGGGCATCCGAAGGAGGACGTCGTCCACTCGGATGGCTAAG |
| | | GGAGAGCTCGGATCCGGCTGCTAACAAAGCCCGAAAGGAAGCTGAG |
| | | TTGGCTGCTGCCACCGCTGAGCAATAACTAGCATAACCCCTTGGGGC |
| | | CTCTAAACGGGTCTTGAGGGGTTTTTTGCTGAAAGGAGGAACTATAT |
| | | CCGGATCGAGATCCTCTAGAGTCGACCTGCAGGCATGCAAGCTTGTA |
| | | TTCTATAGTGTCACCTAAATCGTATGTGTATGATACATAAGGTTATG |
| | | TATTAATTGTAGCCGCGTTCTAACGACAATATGTACAAGCCTAATTG |
| | | TGTAGCATCTGGCTTACTGAAGCAGACCCTATCATCTCTCTCGTAAA |
| | | CTGCCGTCAGAGTCGGTTTGGTTGGACGAACCTTCTGAGTTTCTGGT |
| | | AACGCCGTCCCGCACCCGGAAATGGTCAGCGAACCAATCAGCAGGG |
| | | TCATCGCTAGCCAGATCCTCTACGCCGGACGCATCGTGGCCGGCATC |
| | | ACCGGCGCCACAGGTGCGGTTGCTGGCGCCTATATCGCCGACATCAC |
| | | CGATGGGGAAGATCGGGCTCGCCACTTCGGGCTCATGAGCGCTTGTT |
| | | TCGGCGTGGGTATGGTGGCAGGCCCCGTGGCCGGGGGACTGTTGGG |
| | | CGCCATCTCCTTGCACCATTCCTTGCGGCGGCGGTGCTCAACGGCCT |
| | | CAACCTACTACTGGGCTGCTTCCTAATGCAGGAGTCGCATAAGGGA |
| | | GAGCGTCGAATGGTGCACTCTCAGTACAATCTGCTCTGATGCCGCAT |
| | | AGTTAAGCCAGCCCCGACACCCGCCAACACCCGCTGACGCGCCCTG |
| | | ACGGGCTTGTCTGCTCCCGGCATCCGCTTACAGACAAGCTGTGACCG |
| | | TCTCCGGGAGCTGCATGTGTCAGAGGTTTTCACCGTCATCACCGAAA |
| | | CGCGCGAGACGAAAGGGCCTCGTGATACGCCTATTTTTATAGGTTAA |
| | | TGTCATGATAATAATGGTTTCTTAGACGTCAGGTGGCACTTTTCGGG |
| | | GAAATGTGCGCGGAACCCCTATTTGTTTATTTTTCTAAATACATTCA |
| | | AATATGTATCCGCTCATGAGACAATAACCCTGATAAATGCTTCAATA |
| | | ATATTGAAAAAGGAAGAGTATGAGTATTCAACATTTCCGTGTCGCCC |
| | | TTATTCCCTTTTTTGCGGCATTTTGCCTTCCTGTTTTTGCTCACCCAGA |
| | | AACGCTGGTGAAAGTAAAAGATGCTGAAGATCAGTTGGGTGCACGA |
| | | GTGGGTTACATCGAACTGGATCTCAACAGCGGTAAGATCCTTGAGA |
| | | GTTTTCGCCCCGAAGAACGTTTTCCAATGATGAGCACTTTTAAAGTT |
| | | CTGCTATGTGGCGCGGTATTATCCCGTATTGACGCCGGGCAAGAGCA |
| | | ACTCGGTCGCCGCATACACTATTCTCAGAATGACTTGGTTGAGTACT |
| | | CACCAGTCACAGAAAAGCATCTTACGGATGGCATGACAGTAAGAGA |
| | | ATTATGCAGTGCTGCCATAACCATGAGTGATAACACTGCGGCCAACT |
| | | TACTTCTGACAACGATCGGAGGACCGAAGGAGCTAACCGCTTTTTTG |
| | | CACAACATGGGGGATCATGTAACTCGCCTTGATCGTTGGGAACCGG |
| | | AGCTGAATGAAGCCATACCAAACGACGAGCGTGACACCACGATGCC |
| | | TGTAGCAATGGCAACAACGTTGCGCAAACTATTAACTGGCGAACTA |
| | | CTTACTCTAGCTTCCCGGCAACAATTAATAGACTGGATGGAGGCGGA |
| | | TAAAGTTGCAGGACCACTTCTGCGCTCGGCCCTTCCGGCTGGCTGGT |
| | | TTATTGCTGATAAATCTGGAGCCGGTGAGCGTGGGTCTCGCGGTATC |
| | | ATTGCAGCACTGGGGCCAGATGGTAAGCCCTCCCGTATCGTAGTTAT |
| | | CTACACGACGGGGAGTCAGGCAACTATGGATGAACGAAATAGACAG |
| | | ATCGCTGAGATAGGTGCCTCACTGATTAAGCATTGGTAACTGTCAGA |
| | | CCAAGTTTACTCATATATACTTTAGATTGATTTAAAACTTCATTTTTA |
| | | ATTTAAAAGGATCTAGGTGAAGATCCTTTTTGATAATCTCATGACCA |
| | | AAATCCCTTAACGTGAGTTTTCGTTCCACTGAGCGTCAGACCCCGTA |
| | | GAAAAGATCAAAGGATCTTCTTGAGATCCTTTTTTTCTGCGCGTAAT |
| | | CTGCTGCTTGCAAACAAAAAAACCACCGCTACCAGCGGTGGTTTGTT |
| | | TGCCGGATCAAGAGCTACCAACTCTTTTTCCGAAGGTAACTGGCTTC |
| | | AGCAGAGCGCAGATACCAAATACTGTTCTTCTAGTGTAGCCGTAGTT |
| | | AGGCCACCACTTCAAGAACTCTGTAGCACCGCCTACATACCTCGCTC |
| | | TGCTAATCCTGTTACCAGTGGCTGCTGCCAGTGGCGATAAGTCGTGT |
| | | CTTACCGGGTTGGACTCAAGACGATAGTTACCGGATAAGGCGCAGC |
| | | GGTCGGGCTGAACGGGGGGTTCGTGCACACAGCCCAGCTTGGAGCG |
| | | AACGACCTACACCGAACTGAGATACCTACAGCGTGAGCTATGAGAA |
| | | AGCGCCACGCTTCCCGAAGGGAGAAAGGCGGACAGGTATCCGGTAA |
| | | GCGGCAGGGTCGGAACAGGAGAGCGCACGAGGGAGCTTCCAGGGG |
| | | GAAACGCCTGGTATCTTTATAGTCCTGTCGGGTTTCGCCACCTCTGA |
| | | CTTGAGCGTCGATTTTTGTGATGCTCGTCAGGGGGGCGGAGCCTATG |
| | | GAAAAACGCCAGCAACGCGGCCTTTTTACGGTTCCTGGCCTTTTGCT |
| | | GGCCTTTTGCTCACATGTTCTTTCCTGCGTTATCCCCTGATTCTGTGG |
| | | ATAACCGTATTACCGCCTTTGAGTGAGCTGATACCGCTCGCCGCAGC |
| | | CGAACGACCGAGCGCAGCGAGTCAGTGAGCGAGGAAGCGGAAGAG |
| | | CGCCCAATACGCAAACCGCCTCTCCCCGCGCGTTGGCCGATTCATTA |
| | | ATGCAGGGGGATCTCGATCCCGCGAAATTAATACGACTCACTATAG |
| | | G |
| 7 | pFastBac- | TTCTCTGTCACAGAATGAAAATTTTTCTGTCATCTCTTCGTTATTAAT |
| | GFP-ZEBOV- | GTTTGTAATTGACTGAATATCAACGCTTATTTGCAGCCTGAATGGCG |
| | GP- | AATGGGACGCGCCCTGTAGCGGCGCATTAAGCGCGGCGGGTGTGGT |
| | deltaMLD- | GGTTACGCGCAGCGTGACCGCTACACTTGCCAGCGCCCTAGCGCCCG |
| | SARS-CoV-2- | CTCCTTTCGCTTTCTTCCCTTCCTTTCTCGCCACGTTCGCCGGCTTTCC |
| | RBD | CCGTCAAGCTCTAAATCGGGGGCTCCCTTTAGGGTTCCGATTTAGTG |
| | | CTTTACGGCACCTCGACCCCAAAAAACTTGATTAGGGTGATGGTTCA |
| | | CGTAGTGGGCCATCGCCCTGATAGACGGTTTTTCGCCCTTTGACGTT |
| | | GGAGTCCACGTTCTTTAATAGTGGACTCTTGTTCCAAACTGGAACAA |
| | | CACTCAACCCTATCTCGGTCTATTCTTTTGATTTATAAGGGATTTTGC |
| | | CGATTTCGGCCTATTGGTTAAAAAATGAGCTGATTTAACAAAAATTT |
| | | AACGCGAATTTTAACAAAATATTAACGTTTACAATTTCAGGTGGCAC |
| | | TTTTCGGGGAAATGTGCGCGGAACCCCTATTTGTTTATTTTTCTAAAT |
| | | ACATTCAAATATGTATCCGCTCATGAGACAATAACCCTGATAAATGC |
| | | TTCAATAATATTGAAAAAGGAAGAGTATGAGTATTCAACATTTCCGT |
| | | GTCGCCCTTATTCCCTTTTTTGCGGCATTTTGCCTTCCTGTTTTTGCTC |
| | | ACCCAGAAACGCTGGTGAAAGTAAAAGATGCTGAAGATCAGTTGGG |
| | | TGCACGAGTGGGTTACATCGAACTGGATCTCAACAGCGGTAAGATC |
| | | CTTGAGAGTTTTCGCCCCGAAGAACGTTTTCCAATGATGAGCACTTT |
| | | TAAAGTTCTGCTATGTGGCGCGGTATTATCCCGTATTGACGCCGGGC |
| | | AAGAGCAACTCGGTCGCCGCATACACTATTCTCAGAATGACTTGGTT |
| | | GAGTACTCACCAGTCACAGAAAAGCATCTTACGGATGGCATGACAG |
| | | TAAGAGAATTATGCAGTGCTGCCATAACCATGAGTGATAACACTGC |
| | | GGCCAACTTACTTCTGACAACGATCGGAGGACCGAAGGAGCTAACC |
| | | GCTTTTTTGCACAACATGGGGGATCATGTAACTCGCCTTGATCGTTG |
| | | GGAACCGGAGCTGAATGAAGCCATACCAAACGACGAGCGTGACACC |
| | | ACGATGCCTGTAGCAATGGCAACAACGTTGCGCAAACTATTAACTG |
| | | GCGAACTACTTACTCTAGCTTCCCGGCAACAATTAATAGACTGGATG |
| | | GAGGCGGATAAAGTTGCAGGACCACTTCTGCGCTCGGCCCTTCCGGC |
| | | TGGCTGGTTTATTGCTGATAAATCTGGAGCCGGTGAGCGTGGGTCTC |
| | | GCGGTATCATTGCAGCACTGGGGCCAGATGGTAAGCCCTCCCGTATC |
| | | GTAGTTATCTACACGACGGGGAGTCAGGCAACTATGGATGAACGAA |
| | | ATAGACAGATCGCTGAGATAGGTGCCTCACTGATTAAGCATTGGTA |
| | | ACTGTCAGACCAAGTTTACTCATATATACTTTAGATTGATTTAAAAC |
| | | TTCATTTTTAATTTAAAAGGATCTAGGTGAAGATCCTTTTTGATAATC |
| | | TCATGACCAAAATCCCTTAACGTGAGTTTTCGTTCCACTGAGCGTCA |
| | | GACCCCGTAGAAAAGATCAAAGGATCTTCTTGAGATCCTTTTTTTCT |
| | | GCGCGTAATCTGCTGCTTGCAAACAAAAAAACCACCGCTACCAGCG |
| | | GTGGTTTGTTTGCCGGATCAAGAGCTACCAACTCTTTTTCCGAAGGT |
| | | AACTGGCTTCAGCAGAGCGCAGATACCAAATACTGTCCTTCTAGTGT |
| | | AGCCGTAGTTAGGCCACCACTTCAAGAACTCTGTAGCACCGCCTACA |
| | | TACCTCGCTCTGCTAATCCTGTTACCAGTGGCTGCTGCCAGTGGCGA |
| | | TAAGTCGTGTCTTACCGGGTTGGACTCAAGACGATAGTTACCGGATA |
| | | AGGCGCAGCGGTCGGGCTGAACGGGGGGTTCGTGCACACAGCCCAG |
| | | CTTGGAGCGAACGACCTACACCGAACTGAGATACCTACAGCGTGAG |
| | | CATTGAGAAAGCGCCACGCTTCCCGAAGGGAGAAAGGCGGACAGGT |
| | | ATCCGGTAAGCGGCAGGGTCGGAACAGGAGAGCGCACGAGGGAGC |
| | | TTCCAGGGGGAAACGCCTGGTATCTTTATAGTCCTGTCGGGTTTCGC |
| | | CACCTCTGACTTGAGCGTCGATTTTTGTGATGCTCGTCAGGGGGGCG |
| | | GAGCCTATGGAAAAACGCCAGCAACGCGGCCTTTTTACGGTTCCTGG |
| | | CCTTTTGCTGGCCTTTTGCTCACATGTTCTTTCCTGCGTTATCCCCTG |
| | | ATTCTGTGGATAACCGTATTACCGCCTTTGAGTGAGCTGATACCGCT |
| | | CGCCGCAGCCGAACGACCGAGCGCAGCGAGTCAGTGAGCGAGGAA |
| | | GCGGAAGAGCGCCTGATGCGGTATTTTCTCCTTACGCATCTGTGCGG |
| | | TATTTCACACCGCAGACCAGCCGCGTAACCTGGCAAAATCGGTTACG |
| | | GTTGAGTAATAAATGGATGCCCTGCGTAAGCGGGTGTGGGCGGACA |
| | | ATAAAGTCTTAAACTGAACAAAATAGATCTAAACTATGACAATAAA |
| | | GTCTTAAACTAGACAGAATAGTTGTAAACTGAAATCAGTCCAGTTAT |
| | | GCTGTGAAAAAGCATACTGGACTTTTGTTATGGCTAAAGCAAACTCT |
| | | TCATTTTCTGAAGTGCAAATTGCCCGTCGTATTAAAGAGGGGCGTGG |
| | | CCAAGGGCATGGTAAAGACTATATTCGCGGCGTTGTGACAATTTACC |
| | | GAACAACTCCGCGGCCGGGAAGCCGATCTCGGCTTGAACGAATTGT |
| | | TAGGTGGCGGTACTTGGGTCGATATCAAAGTGCATCACTTCTTCCCG |
| | | TATGCCCAACTTTGTATAGAGAGCCACTGCGGGATCGTCACCGTAAT |
| | | CTGCTTGCACGTAGATCACATAAGCACCAAGCGCGTTGGCCTCATGC |
| | | TTGAGGAGATTGATGAGCGCGGTGGCAATGCCCTGCCTCCGGTGCTC |
| | | GCCGGAGACTGCGAGATCATAGATATAGATCTCACTACGCGGCTGC |
| | | TCAAACCTGGGCAGAACGTAAGCCGCGAGAGCGCCAACAACCGCTT |
| | | CTTGGTCGAAGGCAGCAAGCGCGATGAATGTCTTACTACGGAGCAA |
| | | GTTCCCGAGGTAATCGGAGTCCGGCTGATGTTGGGAGTAGGTGGCT |
| | | ACGTCTCCGAACTCACGACCGAAAAGATCAAGAGCAGCCCGCATGG |
| | | ATTTGACTTGGTCAGGGCCGAGCCTACATGTGCGAATGATGCCCATA |
| | | CTTGAGCCACCTAACTTTGTTTTAGGGCGACTGCCCTGCTGCGTAAC |
| | | ATCGTTGCTGCTGCGTAACATCGTTGCTGCTCCATAACATCAAACAT |
| | | CGACCCACGGCGTAACGCGCTTGCTGCTTGGATGCCCGAGGCATAG |
| | | ACTGTACAAAAAAACAGTCATAACAAGCCATGAAAACCGCCACTGC |
| | | GCCGTTACCACCGCTGCGTTCGGTCAAGGTTCTGGACCAGTTGCGTG |
| | | AGCGCATACGCTACTTGCATTACAGTTTACGAACCGAACAGGCTTAT |
| | | GTCAACTGGGTTCGTGCCTTCATCCGTTTCCACGGTGTGCGTCACCC |
| | | GGCAACCTTGGGCAGCAGCGAAGTCGAGGCATTTCTGTCCTGGCTG |
| | | GCGAACGAGCGCAAGGTTTCGGTCTCCACGCATCGTCAGGCATTGG |
| | | CGGCCTTGCTGTTCTTCTACGGCAAGGTGCTGTGCACGGATCTGCCC |
| | | TGGCTTCAGGAGATCGGTAGACCTCGGCCGTCGCGGCGCTTGCCGGT |
| | | GGTGCTGACCCCGGATGAAGTGGTTCGCATCCTCGGTTTTCTGGAAG |
| | | GCGAGCATCGTTTGTTCGCCCAGGACTCTAGCTATAGTTCTAGTGGTt |
| | | GGCCTACGTACCCGTAGTGGCTATGGCAGGGCTTGCCGCCCCGACGT |
| | | TGGCTGCGAGCCCTGGGCCTTCACCCGAACTTGGGGGTTGGGGTGG |
| | | GGAAAAGGAAGAAACGCGGGCGTATTGGTCCCAATGGGGTCTCGGT |
| | | GGGGTATCGACAGAGTGCCAGCCCTGGGACCGAACCCCGCGTTTAT |
| | | GAACAAACGACCCAACACCCGTGCGTTTTATTCTGTCTTTTTATTGCC |
| | | GTCATAGCGCGGGTTCCTTCCGGTATTGTCTCCTTCCGTGTTTCAGTT |
| | | AGCCTCCCCCATCTCCCGGTACCTTACTTGTACAGCTCGTCCATGCC |
| | | GAGAGTGATCCCGGCGGCGGTCACGAACTCCAGCAGGACCATGTGA |
| | | TCGCGCTTCTCGTTGGGGTCTTTGCTCAGGGCGGACTGGGTGCTCAG |
| | | GTAGTGGTTGTCGGGCAGCAGCACGGGGCCGTCGCCGATGGGGGTG |
| | | TTCTGCTGGTAGTGGTCGGCGAGCTGCACGCTGCCGTCCTCGATGTT |
| | | GTGGCGGGTCTTGAAGTTCACCTTGATGCCGTTCTTCTGCTTGTCGGC |
| | | GGTGATATAGACCTTGTGGCTGTTGTAGTTGTACTCCAGCTTGTGCC |
| | | CCAGGATGTTGCCGTCCTCCTTGAAGTCGATGCCCTTCAGCTCGATG |
| | | CGGTTCACCAGGGTGTCGCCCTCGAACTTCACCTCGGCGCGGGTCTT |
| | | GTAGTTGCCGTCGTCCTTGAAGAAGATGGTGCGCTCCTGGACGTAGC |
| | | CTTCGGGCATGGCGGACTTGAAGAAGTCGTGCTGCTTCATGTGGTCG |
| | | GGGTAGCGGGCGAAGCACTGCACGCCGTAGGTCAAGGTGGTCACGA |
| | | GGGTGGGCCAGGGCACGGGCAGCTTGCCGGTGGTGCAGATGAACTT |
| | | CAGGGTCAGCTTGCCGTAGGTGGCATCGCCCTCGCCCTCGCCGGACA |
| | | CGCTGAACTTGTGGCCGTTTACGTCGCCGTCCAGCTCGACCAGGATG |
| | | GGCACCACCCCGGTGAACAGCTCCTCGCCCTTGCTCACCATCTCGAG |
| | | ATCCCGGGTGATCAAGTCTTCGTCGAGTGATTGTAAATAAAATGTAA |
| | | TTTACAGTATAGTATTTTAATTAATATACAAATGATTTGATAATAATT |
| | | CTTATTTAACTATAATATATTGTGTTGGGTTGAATTAAAGGTCCGTAT |
| | | ACTCCGGAATATTAATAGATCATGGAGATAATTAAAATGATAACCA |
| | | TCTCGCAAATAAATAAGTATTTTACTGTTTTCGTAACAGTTTTGTAAT |
| | | AAAAAAACCTATAAATATTCCGGATTATTCATACCGTCCCACCATCG |
| | | GGCGCGGATCCATGGTAAGCGCTATTGTTTTATATGTGCTTTTGGCG |
| | | GCGGCGGCGCATTCTGCCTTTGCGATTCCTCTTGGCGTTATCCATAAT |
| | | AGTACTCTCCAAGTCAGCGACGTCGATAAACTTGTTTGCCGCGATAA |
| | | ACTTTCCAGTACTAACCAGCTGCGCTCCGTGGGTCTTAACCTCGAAG |
| | | GAAACGGAGTTGCCACCGATGTTCCCTCCGCCACTAAGCGCTGGGG |
| | | CTTCCGCTCCGGAGTCCCACCCAAGGTAGTGAACTATGAGGCGGGC |
| | | GAATGGGCCGAGAACTGTTATAATCTTGAGATAAAAAAACCCGACG |
| | | GATCCGAGTGCCTTCCAGCTGCCCCGGATGGTATTAGGGGCTTCCCA |
| | | CGCTGTCGCTACGTTCATAAAGTATCCGGAACCGGACCTTGTGCTGG |
| | | AGACTTCGCATTCCACAAAGAGGGCGCGTTTTTTCTGTATGATCGCC |
| | | TTGCATCCACCGTAATCTACCGCGGCACGACCTTTGCTGAAGGTGTG |
| | | GTCGCTTTCCTTATTCTCCCACAAGCTAAGAAGGACTTTTTCTCCTCC |
| | | CACCCACTCCGCGAGCCAGTAAACGCAACTGAAGATCCCTCCAGCG |
| | | GTTACTACTCCACCACCATTCGCTACCAGGCAACCGGATTTGGAACA |
| | | AATGAAACAGAGTACCTGTTCGAAGTTGATAATCTGACATACGTCCA |
| | | GCTTGAATCCCGCTTCACTCCCCAGTTTTTGCTGCAACTCAACGAAA |
| | | CAATATACACATCCGGCAAGCGCTCCAACACCACCGGCAAATTGAT |
| | | ATGGAAAGTAAACCCTGAAATTGACACAACTATCGGAGAATGGGCA |
| | | TTCTGGGAAACAAAAAAAAATCTCACTCGCAAAATTAGGTCCGAGG |
| | | AACTCAGCTTCACAGTAGTTTCCAATCGCTTTCCTAATATCACAAAC |
| | | CTGTGCCCATTTGGCGAGGTGTTCAACGCAACCCGCTTCGCCAGCGT |
| | | GTACGCCTGGAATAGGAAGCGGATCAGCAACTGCGTGGCCGACTAT |
| | | AGCGTGCTGTACAACTCCGCCTCTTTCAGCACCTTTAAGTGCTATGG |
| | | CGTGTCCCCCACAAAGCTGAATGACCTGTGCTTTACCAACGTCTACG |
| | | CCGATTCTTTCGTGATCAGGGGCGACGAGGTGCGCCAGATCGCCCCC |
| | | GGCCAGACAGGCAAGATCGCAGACTACAATTATAAGCTGCCAGACG |
| | | ATTTCACCGGCTGCGTGATCGCCTGGAACAGCAACAATCTGGATTCC |
| | | AAAGTGGGCGGCAACTACAATTATCTGTACCGGCTGTTTAGAAAGA |
| | | GCAATCTGAAGCCCTTCGAGAGGGACATCTCTACAGAAATCTACCA |
| | | GGCCGGCAGCACCCCTTGCAATGGCGTGGAGGGCTTTAACTGTTATT |
| | | TCCCACTCCAGTCCTACGGCTTCCAGCCCACAAACGGCGTGGGCTAT |
| | | CAGCCTTACCGCGTGGTGGTGCTGAGCTTTGAGCTGCTGCACGCCCC |
| | | AGCAACAGTGTGCGGCCCCAAGAAGTCCACCAATCTGGTGAAGAAC |
| | | ACCCACCATCAGGACACAGGAGAGGAATCCGCATCCTCCGGTAAAC |
| | | TTGGTTTGATAACAAACACCATCGCAGGCGTAGCTGGACTGATAAC |
| | | AGGCGGACGCCGCACCAGGAGGGAAGCTATTGTCAACGCTCAACCA |
| | | AAATGCAACCCCAACCTTCACTATTGGACCACTCAGGATGAAGGAG |
| | | CCGCTATAGGACTCGCATGGATACCTTACTTCGGTCCCGCAGCTGAA |
| | | GGCATATACACCGAAGGACTGATGCATAACCAAGACGGTCTGATTT |
| | | GTGGTCTTAGGCAGCTGGCAAACGAAACCACTCAGGCCCTGCAGCT |
| | | GTTCTTGCGCGCTACGACAGAACTCCGCACCTTTAGTATCTTGAACC |
| | | GCAAAGCTATTGATTTCCTTCTTCAACGCTGGGGCGGCACCTGCCAC |
| | | ATCCTGGGACCCGATTGCTGTATTGAACCCCACGACTGGACCAAAA |
| | | ACATCACAGACAAGATAGATCAGATAATCCACGATTTTGTAGACAA |
| | | GACCCTCCCAGACCAAGGTGACAATGACAATTGGTGGACAGGTTGG |
| | | CGCCAATGGATCCCTGCAGGCATTGGTGTAACAGGAGTAATCATAG |
| | | CCGTTATCGCCCTGTTTTGTATTCGCAACCGCAACCGCCAGTACTAA |
| | | TCTAGAGCCTGCAGTCTCGACAAGCTTGTCGAGAAGTACTAGAGGA |
| | | TCATAATCAGCCATACCACATTTGTAGAGGTTTTACTTGCTTTAAAA |
| | | AACCTCCCACACCTCCCCCTGAACCTGAAACATAAAATGAATGCAAT |
| | | TGTTGTTGTTAACTTGTTTATTGCAGCTTATAATGGTTACAAATAAAG |
| | | CAATAGCATCACAAATTTCACAAATAAAGCATTTTTTTCACTGCATT |
| | | CTAGTTGTGGTTTGTCCAAACTCATCAATGTATCTTATCATGTCTGGA |
| | | TCTGATCACTGCTTGAGCCTAGGAGATCCGAACCAGATAAGTGAAA |
| | | TCTAGTTCCAAACTATTTTGTCATTTTTAATTTTCGTATTAGCTTACG |
| | | ACGCTACACCCAGTTCCCATCTATTTTGTCACTCTTCCCTAAATAATC |
| | | CTTAAAAACTCCATTTCCACCCCTCCCAGTTCCCAACTATTTTGTCCG |
| | | CCCACAGCGGGGCATTTTTCTTCCTGTTATGTTTTTAATCAAACATCC |
| | | TGCCAACTCCATGTGACAAACCGTCATCTTCGGCTACTTT |

1. A composition comprising a modified viral glycoprotein, wherein one or more portions of the modified viral glycoprotein are modified to comprise an antigen,
wherein the modified glycoprotein comprises a glycoprotein from the family of Filoviridae or mutant thereof,
wherein the antigen comprises a protein or peptide or a variant, homologue, derivative or subsequence thereof from a Coronavirus, and wherein the modified viral glycoprotein and the antigen are from different sources.

2. (canceled)

3. (canceled)

4. (canceled)

5. The composition of claim 1, wherein the modified viral glycoprotein is an Ebola virus glycoprotein or mutant thereof.

6. The composition of claim 1, wherein the one or more portions of the modified viral glycoprotein is a mucin-like domain (MLD) or mutant thereof.

7. The composition of claim 1, wherein the antigen comprises an exogenous antigen, and endogenous antigen, an autoantigen, a neoantigen, a viral antigen, or a tumor antigen.

8. The composition of claim 1, wherein the antigen is an immunogen.

9. The composition of claim 1, wherein the composition elicits, stimulates, induces, promotes, increases or enhances an immune response against both the modified viral glycoprotein and the antigen.

10. The composition of claim 1, wherein the Coronavirus comprises an alphacoronavirus, a betacoronavirus, a gammacoronavirus, or a deltacoronavirus.

11. The composition of claim 1, wherein the Coronavirus is selected from a species or subspecies of Embecovirus, Sarbecovirus, Merbecovirus, Nobevovirus, Hibecovirus, SARS-COV, or MERS-COV.

12. The composition of claim 1, wherein the Coronavirus is selected from a species or subspecies of SARS-COV, SARS-COV-1, SARS-COV-2, MERS-COV, SL-CoV-WIV1, HK84, HKU5, HCoV-OC43, HCoV-HKU1, or HKU9.

13. The composition of claim 1, wherein the Coronavirus comprises an alphacoronavirus, a betacoronavirus, a gammacoronavirus, or a deltacoronavirus protein or peptide or a variant, homologue, derivative or subsequence thereof.

14. The composition of claim 1, wherein the antigen comprises a SARS-COV-2 protein or peptide or a variant, homologue, derivative or subsequence thereof.

15. The composition of claim 1, further comprising an adjuvant.

16.-30. (canceled)

31. The composition of claim 1, further comprising a main glycoprotein trimer.

32. The composition of claim 31, wherein the modified viral glycoprotein is structurally independent from the main glycoprotein trimer.

33. The composition of claim 1, wherein the modified viral glycoprotein is a Marburg virus glycoprotein or mutant thereof.

34. The composition of claim 1, wherein the modified viral glycoprotein is a Sudan ebolavirus glycoprotein or mutant thereof.

35. The composition of claim 1, wherein the modified viral glycoprotein is a Bundibugyo ebolavirus glycoprotein or mutant thereof.

36. A protein-based subunit vaccine comprising the modified viral glycoprotein of claim 1.

37. A viral vector for encoding the modified viral glycoprotein of claim 1.

38. The viral vector of claim 37, wherein the viral vector comprises a recombinant vesicular stomatitis virus (rVSV), a recombinant lentivirus or a recombinant baculovirus.


SEQUENCE LISTING (7 sequences)

SEQ ID NO: 1 - DNA, 2208 bp, Artificial Sequence
  ZEBOV-GP- deltaMLD- SARS-CoV-2- RBD
atgggcgtgactggaatcctgcagctgccaagagatcggttcaagcggacttcatttttcctgtgggtcatcatcctgtttcagagaaccttttctattcctcttggcgttatccataatagtactctccaagtcagcgacgtcgataaacttgtttgccgcgataaactttccagtactaaccagctgcgctccgtgggtcttaacctcgaaggaaacggagttgccaccgatgttccctccgccactaagcgctggggcttccgctccggagtcccacccaaggtagtgaactatgaggcgggcgaatgggccgagaactgttataatcttgagataaaaaaacccgacggatccgagtgccttccagctgccccggatggtattaggggcttcccacgctgtcgctacgttcataaagtatccggaaccggaccttgtgctggagacttcgcattccacaaagagggcgcgttttttctgtatgatcgccttgcatccaccgtaatctaccgcggcacgacctttgctgaaggtgtggtcgctttccttattctcccacaagctaagaaggactttttctcctcccacccactccgcgagccagtaaacgcaactgaagatccctccagcggttactactccaccaccattcgctaccaggcaaccggatttggaacaaatgaaacagagtacctgttcgaagttgataatctgacatacgtccagcttgaatcccgcttcactccccagtttttgctgcaactcaacgaaacaatatacacatccggcaagcgctccaacaccaccggcaaattgatatggaaagtaaaccctgaaattgacacaactatcggagaatgggcattctgggaaacaaaaaaaaatctcactcgcaaaattaggtccgaggaactcagcttcacagtagtttccaatcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacacccaccatcaggacacaggagaggaatccgcatcctccggtaaacttggtttgataacaaacaccatcgcaggcgtagctggactgataacaggcggacgccgcaccaggagggaagctattgtcaacgctcaaccaaaatgcaaccccaaccttcactattggaccactcaggatgaaggagccgctataggactcgcatggataccttacttcggtcccgcagctgaaggcatatacaccgaaggactgatgcataaccaagacggtctgatttgtggtcttaggcagctggcaaacgaaaccactcaggccctgcagctgttcttgcgcgctacgacagaactccgcacctttagtatcttgaaccgcaaagctattgatttccttcttcaacgctggggcggcacctgccacatcctgggacccgattgctgtattgaaccccacgactggaccaaaaacatcacagacaagatagatcagataatccacgattttgtagacaagaccctcccagaccaaggtgacaatgacaattggtggacaggttggcgccaatggatccctgcaggcattggtgtaacaggagtaatcatagccgttatcgccctgttttgtatttgtaaattcgtgttttga

SEQ ID NO: 2 - DNA, 2133 bp, Artificial Sequence
  MARV-GP- deltaMLD- SARS-CoV-2- RBD
atgaaaaccacctgcctgctgatcagcctgatcctgatccagggcgtgaaaaccctgcccatcctggaaatcgccagcaacatccagccccagaacgtggacagcgtgtgcagcggcaccctgcagaaaaccgaggacgtgcacctgatgggcttcacactgagcggccagaaggtggccgacagccccctggaagccagcaagagatgggccttcagagccggcgtgccccccaagaatgtggagtacaccgaaggcgaggaagccaagacctgctacaacatcagcgtgaccgaccccagcggcaagagcctgctgctggacccccccaccaacatcagagactaccccaagtgcaagaccatccaccacatccagggccagaacccccacgcccagggcattgctctgcacctgtggggcgcctttttcctgtacgaccggatcgccagcaccaccatgtaccggggcaaggtgttcaccgagggcaatatcgccgccatgatcgtgaacaagaccgtgcacaagatgatcttcagcagacagggccagggctaccggcacatgaacctgaccagcaccaacaagtactggaccagcagcaacggcacccagaccaacgacaccggctgcttcggcacactgcaggaatacaacagcaccaagaaccagacctgcgcccccagcaagaagcccctgcccctgcctaccgcccaccccgaagtgaagctgacctccacctccaccgacgccaccaagctgaacaccaccgaccccaacagcgacgacgaggacctgaccacctccggcagcggcagcggcgagcagcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagatcgacttcgaccccgtgcctaacaccaagaccatcttcgacgaatcttcatcttccggagcttcagctgaagaggaccagcacgccagccccaacatctccctgaccctgagctacttccccaaagtgaacgagaacaccgcccacagcggcgagaacgagaacgactgcgacgccgagctgcggatttggagcgtgcaggaagatgacctggccgccggactgagctggatccccttcttcggccctggcatcgagggcctgtacacagccggcctgattaagaaccagaacaacctggtgtgccggctgcggcggctggccaaccagaccgccaagtccctggaactgctgctgagagtgaccaccgaggaacggaccttctccctgatcaaccggcacgccatcgattttctgctggccagatggggcggcacctgcaaggtgctgggccccgactgctgcatcggcatcgaggatctgagccggaacatcagcgagcagatcgaccagatcaagaaggacgagcagaaagagggcaccggctggggcctgggcggcaagtggtggaccagcgactggggcgtgctgaccaacctgggcatcctgctgctgctgtctatcgccgtgctgatcgccctgagctgcatctgccggatcttcaccaagtacatcggctga

SEQ ID NO: 3 - DNA, 2211 bp, Artificial Sequence
  SUDV-GP- deltaMLD- SARS-CoV-1- RBD
atggggggtcttagcctactccaattgcccagggacaaatttcggaaaagctctttctttgtttgggtcatcatcttattccaaaaggccttttccatgcctttgggtgttgtgactaacagcactttagaagtaacagagattgaccagctagtctgcaaggatcatcttgcatctactgaccagctgaaatcagttggtctcaacctcgaggggagcggagtatctactgatatcccatctgcaacaaagcgttggggcttcagatctggtgttcctcccaaggtggtcagctatgaagcgggagaatgggctgaaaattgctacaatcttgaaataaagaagccggacgggagcgaatgcttacccccaccgccagatggtgtcagaggctttccaaggtgccgctatgttcacaaagcccaaggaaccgggccctgcccaggtgactacgcctttcacaaggatggagctttcttcctctatgacaggctggcttcaactgtaatttacagaggagtcaattttgctgagggggtaattgcattcttgatattggctaaaccaaaagaaacgttccttcagtcaccccccattcgagaggcagtaaactacactgaaaatacatcaagttattatgccacatcctacttggagtatgaaatcgaaaattttggtgctcaacactccacgacccttttcaaaattgacaataatacttttgttcgtctggacaggccccacacgcctcagttccttttccagctgaatgataccattcaccttcaccaacagttgagtaatacaactgggagactaatttggacactagatgctaatatcaatgctgatattggtgaatgggctttttgggaaaataaaaagaatctctccgaacaactacgtggagaagagctgtctttcgaagctttatcgctccgcttcccaaatatcaccaacctgtgccccttcggcgaggtgtttaatgccacaaagttccccagcgtgtacgcctgggagcgcaagaagatcagcaactgcgtggccgactactccgtgctgtataatagcaccttcttcagcaccttcaagtgctacggcgtgagcgccaccaagctgaatgacctgtgcttctctaacgtgtatgccgatagctttgtggtgaagggcgacgatgtgaggcagatcgcacctggacagaccggcgtgatcgcagactacaactataagctgccagacgatttcatgggctgcgtgctggcctggaatacacgcaacatcgatgccacatccaccggcaactacaattataagtaccggtatctgagacacggcaagctgcggcccttcgagagagacatctccaatgtgccattttctccagatggcaagccatgcaccccacctgccctgaattgttactggcctctgaacgactacggcttctataccacaaccggcatcggctaccagccttatagggtggtggtgctgtcctttgagctgctgaacgcacctgcaaccgtgtgcggaccaaagctgtctaccgacctgatcaagaatcagaatataacaacagcggttaaaactgtcctgccacaggagtccacaagcaacggtctaataacttcaacagtaacagggattcttgggagtcttgggcttcgaaaacgcagcagaagacaaactaacaccaaagccacgggtaagtgcaatcccaacttacactactggactgcacaagaacaacataatgctgctgggattgcctggatcccgtactttggaccgggtgcggaaggcatatacactgaaggcctgatgcataaccaaaatgccttagtctgtggacttaggcaacttgcaaatgaaacaactcaagctctgcagcttttcttaagagccacaacggagctgcggacatataccatactcaataggaaggccatagatttccttctgcgacgatggggcgggacatgcaggatcctgggaccagattgttgcattgagccacatgattggacaaaaaacatcactgataaaatcaaccaaatcatccatgacttcatcgacaaccccttacctaatcaggataatgatgataattggtggacgggctggagacagtggatccctgcaggaataggcattactggaattattattgcaattattgctcttctttgcgtttgcaagctgctttgctga

SEQ ID NO: 4 - DNA, 2253 bp, Artificial Sequence
  BDBV-GP- deltaMLD- MERS-CoV- RBD
atggtgaccagcggcatcctgcagctgccccgggagcggttcagaaagaccagcttcttcgtgtgggtgatcatcctgttccacaaggtgttccccatccccctgggagtcgtccacaacaacaccctgcaggtctccgacatcgacaagctggtgtgccgggacaaactgtcatccacaagccagctgaagtccgtgggcctgaacctggaaggcaacggagtcgctacagatgtccccaccgccaccaagagatggggcttcagagccggcgtgccccccaaggtggtgaactacgaggccggcgagtgggccgagaactgctacaacctggacatcaagaaggccgacggcagcgagtgtctgcctgaggcccccgagggagtgagaggcttcccccggtgcagatacgtgcacaaggtgtccggcaccggcccttgccctgagggctacgccttccacaaagagggcgcctttttcctgtacgacagactggcttccaccatcatctacagaagcaccaccttcagcgaaggagtcgtcgccttcctgatcctgcccgagacaaagaaggacttcttccagagcccccctctgcacgagcccgccaacatgaccaccgaccccagcagctactaccacaccgtgaccctgaattacgtggccgacaacttcggcaccaatatgaccaactttctgttccaggtggaccatctgacctacgtgcagctggaaccccggttcaccccccagtttctggtgcagctgaacgagacaatctacaccaacggcaggcggagcaacaccaccggcaccctgatctggaaagtgaaccccaccgtggacaccggcgtgggcgaatgggccttctgggagaacaagaagaacttcaccaagaccctgagcagcgaggaactgagcgtgatcttcgtgcccagacaggcagagggagtggagtgcgacttctccccactgctgtctggcacaccacctcaggtgtataatttcaagaggctggtgtttacaaactgtaattacaacctgaccaagctgctgtccctgttctctgtgaacgacttcacctgctcccagatcagccctgccgccatcgcctccaattgttatagctccctgatcctggattacttctcttatcccctgtctatgaagagcgacctgtccgtgtctagcgccggccctatcagccagtttaattacaagcagtccttctctaaccccacatgcctgatcctggccaccgtgcctcacaacctgaccacaatcacaaagccactgaagtactcctatatcaataagtgcagcaggctgctgtccgacgaccgcaccgaggtgcctcagctggtgaatgccaaccagtactctccatgcgtgagcatcgtgccatccaccgtgtgggaggacggcgattactatagaaagcagctgagcccactggagggaggaggatggctggtggcaagcggctccacagtggccatgaccgagcagctccagatgggcttcggcatcacagtgcagtacggcacagataccaatagcgtgtgccccaagctggagtttgccaacgacaccaagatcgcctcccagaccgacagcaaccggcccaaccccatcgacatcagcgagagcaccgagcctggacctctgacaaatacaacaagaggagctgctaatctgctgaccggcagccggcggaccagacgggagatcaccctgcggacccaggccaagtgcaaccccaatctgcactactggaccacccaggacgagggagccgccatcggcctggcctggatcccttacttcggccctgccgccgagggcatctacacagagggcatcatgcacaaccagaacggcctgatctgcggcctgcggcagctggccaacgagacaacccaggccctgcagctgttcctgcgggccaccaccgagctgcggaccttcagcatcctgaacagaaaggccatcgactttctgctgcagcgctggggcggcacctgtcacatcctgggccccgactgctgcatcgagccccacgactggaccaagaacatcaccgacaagatcgaccagatcatccacgacttcatcgataagcccctgcccgaccagaccgacaacgacaattggtggacaggctggcggcagtgggtgccagccggcatcggcatcaccggcgtgatcattgccgtgatcgccctgctgtgcatctgcaagtttctgctgtga

SEQ ID NO: 5 - DNA, 2307 bp, Artificial Sequence
  ZEBOV-GP- deltaMLD- mCherry
atgggcgtgactggaatcctgcagctgccaagagatcggttcaagcggacttcatttttcctgtgggtcatcatcctgtttcagagaaccttttctattcctcttggcgttatccataatagtactctccaagtcagcgacgtcgataaacttgtttgccgcgataaactttccagtactaaccagctgcgctccgtgggtcttaacctcgaaggaaacggagttgccaccgatgttccctccgccactaagcgctggggcttccgctccggagtcccacccaaggtagtgaactatgaggcgggcgaatgggccgagaactgttataatcttgagataaaaaaacccgacggatccgagtgccttccagctgccccggatggtattaggggcttcccacgctgtcgctacgttcataaagtatccggaaccggaccttgtgctggagacttcgcattccacaaagagggcgcgttttttctgtatgatcgccttgcatccaccgtaatctaccgcggcacgacctttgctgaaggtgtggtcgctttccttattctcccacaagctaagaaggactttttctcctcccacccactccgcgagccagtaaacgcaactgaagatccctccagcggttactactccaccaccattcgctaccaggcaaccggatttggaacaaatgaaacagagtacctgttcgaagttgataatctgacatacgtccagcttgaatcccgcttcactccccagtttttgctgcaactcaacgaaacaatatacacatccggcaagcgctccaacaccaccggcaaattgatatggaaagtaaaccctgaaattgacacaactatcggagaatgggcattctgggaaacaaaaaaaaatctcactcgcaaaattaggtccgaggaactcagcttcacagtagtttccaatggcagcggagtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggcagcggaaacacccaccatcaggacacaggagaggaatccgcatcctccggtaaacttggtttgataacaaacaccatcgcaggcgtagctggactgataacaggcggacgccgcaccaggagggaagctattgtcaacgctcaaccaaaatgcaaccccaaccttcactattggaccactcaggatgaaggagccgctataggactcgcatggataccttacttcggtcccgcagctgaaggcatatacaccgaaggactgatgcataaccaagacggtctgatttgtggtcttaggcagctggcaaacgaaaccactcaggccctgcagctgttcttgcgcgctacgacagaactccgcacctttagtatcttgaaccgcaaagctattgatttccttcttcaacgctggggcggcacctgccacatcctgggacccgattgctgtattgaaccccacgactggaccaaaaacatcacagacaagatagatcagataatccacgattttgtagacaagaccctcccagaccaaggtgacaatgacaattggtggacaggttggcgccaatggatccctgcaggcattggtgtaacaggagtaatcatagccgttatcgccctgttttgtatttgtaaattcgtgttttga

SEQ ID NO: 6 - DNA, 15734 bp, Artificial Sequence
  pVenus- ZEBOV-GP- deltaMLD- SARS-CoV-2- RBD
acgaagacaaacaaaccattattatcattaaaaggctcaggagaaactttaacagtaatcagaattctcgaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttaatctgcaccaccggcaagctgcccgtgccctggcccaccctcgtgaccaccttaggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcacggccgacaagcagaagaacggcatcaaggcgaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaaagcggccgccctcctcaggtgctggccatatgaaaaaaactaacagtaatcaaaatgtctgttacagtcaagagaatcattgacaacacagtcatagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagatatcatggataatctcacaaaagttcgtgagtatctcaagtcctactctcgtctagatcaggcggtaggagagatagatgagatcgaagcacaacgagctgaaaagtccaattatgagttgttccaagaggacggagtggaagagcatactaggccctcttattttcaggcagcagatgattctgacacagaatctgaaccagaaattgaagacaatcaaggcttgtatgtaccagatccggaagctgagcaagttgaaggctttatacaggggcctttagatgactatgcagatgaggacgtggatgttgtattcacttcggactggaaacagcctgagcttgaatccgacgagcatggaaagaccttacggttgacattgccagagggtttaagtggagagcagaaatcccagtggcttttgacgattaaagcagtcgttcaaagtgccaaacactggaatctggcagagtgcacatttgaagcatcgggagaaggggtcatcataaaaaagcgccagataactccggatgtatataaggtcactccagtgatgaacacacatccgtaccaatcagaagccgtatcagatgtttggtctctctcaaagacatccatgactttccaacccaagaaagcaagtcttcagcctctcaccatatccttggatgaattgttctcatctagaggagaattcatctctgtcggaggtaacggacgaatgtctcataaagaggccatcctgctcggtctgaggtacaaaaagttgtacaatcaggcgagagtcaaatattctctgtagactatgaaaaaaagtaacagatatcacaatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacgctcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggattctatcagccacttcaaatgagctagtctagcttccagcttctgaacaatccccggtttactcagtctctcctaattccagcctttcgaacaactaatatcctgtcttttctatccctatgaaaaaaactaacagagatcgatctgtttccttgacacgcgtggcccaggcggcccacgatgggcgtgactggaatcctgcagctgccaagagatcggttcaagcggacttcatttttcctgtgggtcatcatcctgtttcagagaaccttttctattcctcttggcgttatccataatagtactctccaagtcagcgacgtcgataaacttgtttgccgcgataaactttccagtactaaccagctgcgctccgtgggtcttaacctcgaaggaaacggagttgccaccgatgttccctccgccactaagcgctggggcttccgctccggagtcccacccaaggtagtgaactatgaggcgggcgaatgggccgagaactgttataatcttgagataaaaaaacccgacggatccgagtgccttccagctgccccggatggtattaggggcttcccacgctgtcgctacgttcataaagtatccggaaccggaccttgtgctggagacttcgcattccacaaagagggcgcgttttttctgtatgatcgccttgcatccaccgtaatctaccgcggcacgacctttgctgaaggtgtggtcgctttccttattctcccacaagctaagaaggactttttctcctcccacccactccgcgagccagtaaacgcaactgaagatccctccagcggttactactccaccaccattcgctaccaggcaaccggatttggaacaaatgaaacagagtacctgttcgaagttgataatctgacatacgtccagcttgaatcccgcttcactccccagtttttgctgcaactcaacgaaacaatatacacatccggcaagcgctccaacaccaccggcaaattgatatggaaagtaaaccctgaaattgacacaactatcggagaatgggcattctgggaaacaaaaaaaaatctcactcgcaaaattaggtccgaggaactcagcttcacagtagtttccaatcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacacccaccatcaggacacaggagaggaatccgcatcctccggtaaacttggtttgataacaaacaccatcgcaggcgtagctggactgataacaggcggacgccgcaccaggagggaagctattgtcaacgctcaaccaaaatgcaaccccaaccttcactattggaccactcaggatgaaggagccgctataggactcgcatggataccttacttcggtcccgcagctgaaggcatatacaccgaaggactgatgcataaccaagacggtctgatttgtggtcttaggcagctggcaaacgaaaccactcaggccctgcagctgttcttgcgcgctacgacagaactccgcacctttagtatcttgaaccgcaaagctattgatttccttcttcaacgctggggcggcacctgccacatcctgggacccgattgctgtattgaaccccacgactggaccaaaaacatcacagacaagatagatcagataatccacgattttgtagacaagaccctcccagaccaaggtgacaatgacaattggtggacaggttggcgccaatggatccctgcaggcattggtgtaacaggagtaatcatagccgttatcgccctgttttgtatttgtaaattcgtgttttgaggccctcgcggccgccctgcacaacagattcttcatgtttgaaccaaatcaacttgtgatatcatgctcaaagaggccttaattatattttaatttttaatttttatgaaaaaaactaacagcaatcatggaagtccacgattttgagaccgacgagttcaatgatttcaatgaagatgactatgccacaagagaattcctgaatcccgatgagcgcatgacgtacttgaatcatgctgattacaatttgaattctcctctaattagtgatgatattgacaatttgatcaggaaattcaattctcttccgattccctcgatgtgggatagtaagaactgggatggagttcttgagatgttaacatcatgtcaagccaatcccatctcaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggagtatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttggcagttatccagcaatcatttccgattaggtgggaggctatttcagtaaaaggaggatacaagcagaagtggagtactagaggtgatgggctcccaaaagatacccgaatttcagactccttggccccaatcgggaactggatcagatctttggaattggtccgaaaccaagttcgtctaaatccattcaataagatcttgttcaatcagctatgtcgtacagtggataatcatttgaagtggtcaaatttgcgaaaaaacacaggaatgattgaatggatcaatgggcgaatttcaaaagaagaccggtctatactgatgttgaagagtgacctacatgaggaaaactcttggagagattaaaaaatcaggaggagactccaaactttaagtatgaaaaaaactttgatccttaagaccctcttgtggtttttatttttttatctggttttgtggtcttcgtgggtcggcatggcatctccacctcctcgcggtccgacctgggcatccgaaggaggacgtcgtccactcggatggctaagggagagctcggatccggctgctaacaaagcccgaaaggaagctgagttggctgctgccaccgctgagcaataactagcataaccccttggggcctctaaacgggtcttgaggggttttttgctgaaaggaggaactatatccggatcgagatcctctagagtcgacctgcaggcatgcaagcttgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagggggatctcgatcccgcgaaattaatacgactcactatagg

SEQ ID NO: 7 - DNA, 8039 bp, Artificial Sequence
  pFastBac- GFP-ZEBOV- GP- deltaMLD- SARS-CoV-2- RBD
ttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggcctacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcgggtcttgaagttcaccttgatgccgttcttctgcttgtcggcggtgatatagaccttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccgtaggtcaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactccggaatattaatagatcatggagataattaaaatgataaccatctcgcaaataaataagtattttactgttttcgtaacagttttgtaataaaaaaacctataaatattccggattattcataccgtcccaccatcgggcgcggatccatggtaagcgctattgttttatatgtgcttttggcggcggcggcgcattctgcctttgcgattcctcttggcgttatccataatagtactctccaagtcagcgacgtcgataaacttgtttgccgcgataaactttccagtactaaccagctgcgctccgtgggtcttaacctcgaaggaaacggagttgccaccgatgttccctccgccactaagcgctggggcttccgctccggagtcccacccaaggtagtgaactatgaggcgggcgaatgggccgagaactgttataatcttgagataaaaaaacccgacggatccgagtgccttccagctgccccggatggtattaggggcttcccacgctgtcgctacgttcataaagtatccggaaccggaccttgtgctggagacttcgcattccacaaagagggcgcgttttttctgtatgatcgccttgcatccaccgtaatctaccgcggcacgacctttgctgaaggtgtggtcgctttccttattctcccacaagctaagaaggactttttctcctcccacccactccgcgagccagtaaacgcaactgaagatccctccagcggttactactccaccaccattcgctaccaggcaaccggatttggaacaaatgaaacagagtacctgttcgaagttgataatctgacatacgtccagcttgaatcccgcttcactccccagtttttgctgcaactcaacgaaacaatatacacatccggcaagcgctccaacaccaccggcaaattgatatggaaagtaaaccctgaaattgacacaactatcggagaatgggcattctgggaaacaaaaaaaaatctcactcgcaaaattaggtccgaggaactcagcttcacagtagtttccaatcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacacccaccatcaggacacaggagaggaatccgcatcctccggtaaacttggtttgataacaaacaccatcgcaggcgtagctggactgataacaggcggacgccgcaccaggagggaagctattgtcaacgctcaaccaaaatgcaaccccaaccttcactattggaccactcaggatgaaggagccgctataggactcgcatggataccttacttcggtcccgcagctgaaggcatatacaccgaaggactgatgcataaccaagacggtctgatttgtggtcttaggcagctggcaaacgaaaccactcaggccctgcagctgttcttgcgcgctacgacagaactccgcacctttagtatcttgaaccgcaaagctattgatttccttcttcaacgctggggcggcacctgccacatcctgggacccgattgctgtattgaaccccacgactggaccaaaaacatcacagacaagatagatcagataatccacgattttgtagacaagaccctcccagaccaaggtgacaatgacaattggtggacaggttggcgccaatggatccctgcaggcattggtgtaacaggagtaatcatagccgttatcgccctgttttgtattcgcaaccgcaaccgccagtactaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctacttt